(19) United States
(12) Patent Application Publication
(10) Pub. No.: US 2022/0184008 A1
(43) Pub. Date: Jun. 16, 2022

(54) MODULATORS OF INTRACELLULAR CHLORIDE CONCENTRATION

(71) Applicants: FONDAZIONE ISTITUTO ITALIANO DI TECNOLOGIA, Genova (IT); FONDAZIONE TELETHON, Roma (IT); ALMA MATER STUDIORUM - UNIVERSITA' DI BOLOGNA, Bologna (IT); UNIVERSITA' DEGLI STUDIO DI GENOVA, Genova (IT)

(72) Inventors: Laura Cancedda, Genova (IT); Marco De Vivo, Genova (IT); Andrea Contestabile, Genova (IT); Marco Borgogno, Bologna (IT); Annalisa Savardi, Genova (IT); Jose Antonio Ortega Martinez, Genova (IT)

(21) Appl. No.: 17/594,070

(22) PCT Filed: Apr. 2, 2020
(86) PCT No.: PCT/IB2020/053158

§ 371 Date: Oct. 1, 2021

(30) Foreign Application Priority Data
Apr. 2, 2019 (IT) 102019000004929

(51) Int. Cl. A61K 31/18; A61K 45/06; A61P 25/00; A61P 25/28; C07C 311/39; C07D 307/52; C07C 311/43; C07D 295/26; C07D 309/14; C07D 211/96; A61K 31/196; A61K 31/341; A61K 31/40; A61K 31/4453; A61K 31/5375; A61K 31/495; A61K 31/445; A61K 31/351

(52) U.S. Cl. CPC A61K 31/18; A61K 31/351; A61P 25/00; A61P 25/28; C07C 311/39; C07D 307/52; C07C 311/43; C07D 295/26; C07D 309/14; C07D 211/96; A61K 31/196; A61K 31/341; A61K 31/40; A61K 31/4453; A61K 31/5375; A61K 31/495; A61K 31/445; A61K 45/06

(57) ABSTRACT

The present invention relates to a compound of Formula la, lb and Ic, (Formula Ia) a pharmaceutical composition comprising the same and their use in the treatment or prevention of pathological conditions associated to depolarizing GABAergic transmission including, for example, Down syndrome and autism.

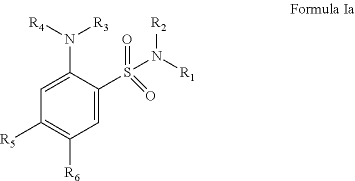

### CROSS-REFERENCE TO RELATED APPLICATIONS

This patent application claims priority from Italian patent application no. 102019000004929 filed on Apr. 2, 2019, the entire disclosure of which is incorporated herein by reference.

### TECHNICAL FIELD

The present invention relates to a compound of Formula Ia, Ib and Ic that inhibits the sodium, potassium and chloride cotransporter (here below also referred to as NKCC1).

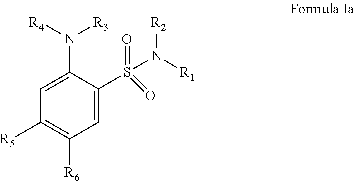

Pharmacological inhibition of NKCC1 can be used to treat a variety of pathophysiological conditions, especially brain disorders. 2-aminobenzenesulfonamide derivatives are potent NKCC1 inhibitors and display promising efficacy in restoring GABAergic transmission and related cognitive behaviors in rodent models of Down syndrome and autism.

### BACKGROUND

Down syndrome is the most common genetic form of intellectual disability (˜10 in 10,000 and 14 in 10,000 live births in European countries and the United States, respectively). Down syndrome, also known as trisomy 21, is a genetic disorder caused by the presence of all, or of part, of a third copy of chromosome 21. The most striking clinical features of Down syndrome are intellectual disabilities, characterized by low Intelligence Quotient (IQ), learning deficits, and memory impairment, particularly in hippocampus-related functions. Although pedagogic methods and educational mainstreaming have led to an improvement in cognitive development in those who have Down syndrome, still there are constitutive impairments that cannot be fully addressed by said methodologies. Indeed, even though there are several clinical candidates to treat Down syndrome (namely piracetam, memantine and donepezil, rivastigmine, epigallocatechin gallate and antioxidants, pentylenetrazol, ACI-24), there are still no approved pharmacological drugs to ameliorate the cognitive symptoms of Down syndrome. Thus, efforts to discover drugs for enhancing cognitive functions in Down syndrome subjects are urgently needed.

In the last few years, a large body of literature has indicated that inhibitory GABAergic transmission via Cl-permeable GABAA receptors is defective in Down syndrome and in many other neurodevelopmental diseases (Deidda, G. et al. Modulation of GABAergic transmission in development and neurodevelopmental disorders: investigating physiology and pathology to gain therapeutic perspectives. Front Cell Neurosci 2014, 8, 119.3; Contestabile, A. et al. The GABAergic Hypothesis for Cognitive Disabilities in Down syndrome. Frontiers in Cellular Neurosciences 2017, 11.54). Nevertheless, it is dangerous to use common GABAA receptor inhibitors to restore defective GABAergic transmission. This is due to the high risk of epileptic seizures in patients.

Brain disorders characterized by altered GABAergic transmission comprise Down syndrome, neuropathic pain, stroke, cerebral ischemia, cerebral edema, hydrocephalus, traumatic brain injury, Brain Trauma-Induced Depressive-Like Behavior, autism spectrum disorders (i.e. autism, Fragile X, Rett, Asperger and DiGeorge syndromes), epilepsy, seizures, epileptic state, childhood spasms, glioma, glioblastoma, anaplastic astrocytoma, Parkinson's disease, Huntington's disease, schizophrenia, anxiety, Tuberous Sclerosis Complex and associated behavioural problems, Dravet syndrome. Na⁺, K⁺, Cl⁻ cotransporters (NKCC) encoded by the SLC12A2 (NKCC1) and SLC12A1 (NKCC2) genes, belong to a family of transporters which provide electroneutral transport of sodium, potassium and chloride across the plasma membrane; they move each solute in the same direction and maintain electroneutrality by moving two positively charged solutes (sodium and potassium) alongside two parts of a negatively charged solute (chloride).

NKCC1 is widely distributed, especially in exocrine glands and brain; NKCC2 is found in the kidney, where it serves to extract sodium, potassium, and chloride from the urine so that they can be reabsorbed into the blood. In neurons, the Cl⁻ importer NKCC1 and the Cl⁻ exporter KCC2 mainly control intracellular Cl⁻ concentration. Importantly, the NKCC1/KCC2 expression ratio is defective in Down syndrome and in several animal models of brain diseases; targeting NKCC1 with inhibitors results in therapeutic effects for several diseases, including without limitations Down syndrome, neuropathic pain, stroke, cerebral ischemia, cerebral edema, hydrocephalus, traumatic brain injury, Brain Trauma-Induced Depressive-Like Behavior, autism spectrum disorders (i.e. autism, Fragile X, Rett, Asperger and DiGeorge syndromes), epilepsy, seizures, epileptic state, childhood spasms, glioma, glioblastoma, anaplastic astrocytoma, Parkinson's disease, Huntington's disease, schizophrenia, anxiety, Tuberous Sclerosis Complex and associated behavioral problems, Dravet syndrome. In animal models, NKCC1 inhibition by the FDA-approved diuretic bumetanide rescues behavioral deficits. Notably, bumetanide restored GABAAR-driven Cl⁻ currents, synaptic plasticity and hippocampus-dependent memory in adult Down syndrome mice models. Hence, NKCC1 inhibitors have shown to have therapeutic activity in diseases where GABAergic transmission is defective.

Moreover, in five independent clinical studies (including a phase II clinical trial), bumetanide treatment reduced autism childhood ratings and emotional face perception.

Nevertheless, bumetanide has a diuretic effect because it also inhibits the kidney-specific Cl⁻ transporter NKCC2. This diuretic effect generates an ionic imbalance and seriously jeopardizes drug compliance during chronic treatment.

Diseases in which Bumetanide has been shown to have an ameliorative effect include Down syndrome, neuropathic pain, stroke, cerebral ischemia, cerebral edema, hydrocephalus, traumatic brain injury, Brain Trauma-Induced Depressive-Like Behavior, autism spectrum disorders (i.e. autism, Fragile X, Rett, Asperger and DiGeorge syndromes), epilepsy, seizures, epileptic state, childhood spasms, glioma, glioblastoma, anaplastic astrocytoma, Parkinson's disease, Hungtinton's disease, schizophrenia, anxiety, Tuberous Sclerosis Complex and associated behavioral problems, Dravet syndrome.

WO 2010/085352 describes the use of NKCC1 modulators in order to improve the cognitive performance of subjects in need thereof. It is also alleged that these compounds can be used in long-term treatments due to the reduction of the unwanted diuretic effect. The most promising compound, 3-Aminosulfonyl-5-N,N-dibutylamino-4-phenoxybenzoic acid, is described to interact with the GABAA receptor, therefore it is neither a NKCC1 nor a NKCC2 inhibitor and potentially presents the risk of undesired side effects including epileptic seizures.

WO 2014/076235 describes compounds for the treatment of the X fragile syndrome. In a preferred embodiment, the chloride modulator is a selective inhibitor of NKCC1.

In the publication of Huang et al. (“Novel NKCC1 Inhibitors Reduces Stroke Damages; Stroke, April, 2019) it is investigated the efficacy of STS66, a 3-(butylamino)-2-phenoxy-benzenesulfonamide. This compound is a close analogue and derivative of bumetanide, thus acting as a NKCC1 inhibitor.

Lykke et al., in “The search for NKCC1-selective drugs for the treatment of epilepsy: Structure-function relationship of bumetanide and various bumetanide derivatives in inhibiting the human cation-chloride cotransporter NKCC1A.” Epilepsy & Behavior 59 (2016) 42-49, investigate bumetanide derivatives as selective inhibitors of NKCC1. The tested derivatives were chosen from ˜5000 3-amino-5-sulfamoylbenzoic acid derivatives that were synthesized in the 1960s and 1970s at Leo Pharma by Peter W. Feit and colleagues during screening for compounds with high diuretic efficacy, finally resulting in the discovery of bumetanide. According to the authors, none of the compounds exerted a markedly higher NKCC2/NKCC1 selectivity. The authors conclude that it will be difficult, if not impossible, to develop bumetanide derivatives with higher selectivity than bumetanide for NKCC1 vs. NKCC2.

Thus, there is a need for alternative therapeutic approaches for Down syndrome and other brain disorders enabling restoration of defective GABAergic transmission through inhibition of NKCC1.

As such, bumetanide is not a viable therapeutic strategy and the same is true for the described analogues. There still exists a strong need of alternative compounds.

### SUMMARY OF THE INVENTION

The invention relates to novel 2-aminobenzenesulfonamide derivatives that inhibit the sodium, potassium and chloride cotransporter (herein also referred to as NKCC1). Pharmacological inhibition of NKCC1 can be used to treat a variety of pathophysiological conditions, especially brain disorders. The modulation of NKCC1 results in fine tuning of GABAergic transmission, hence NKCC1 inhibitors have beneficial effect in diseases characterized by defective NKCC1/KCC2 expression ratio and/or defective GABAergic transmission via Cl⁻ permeable GABAA receptors. A purpose of the present invention to treat diseases with GABA A involvement and/or chloride homeostasis involvement.

### OBJECT OF THE INVENTION

As per a first object, the present invention provides new 2-aminobenzenesulfonamide derivatives capable of inhibiting the sodium, potassium and chloride cotransporter (also briefly referred to as NKCC1).

The present invention discloses as well a process for the preparation of the disclosed compounds.

In a second object, there is disclosed the use of compounds of the invention for the treatment or prevention of pathological conditions associated to the depolarization of GABAergic transmission.

Pharmaceutical preparations comprising the compounds of the invention represent a third object of the invention. In a fourth object, there is disclosed a method for the treatment or prevention of pathological conditions associated to the depolarization of the GABAergic transmission comprising the administration of the compounds of the invention to a patient in need thereof.

### BRIEF DISCLOSURE OF THE FIGURES

FIG. 1: In vitro testing of the NKCC1 inhibitors in the chloride kinetic assay a) Example traces obtained in the chloride kinetic assay on HEK cells transfected with the YFP (mock) or with YFP and NKCC1. The arrow indicates the addition of NaCl (final concentration 74 mM) used to initiate the flux assay. b) Quantification of the effect of bumetanide (10 μM and 100 μM) or furosemide (10 μM and 100 μM) in the chloride kinetic assay on mock or NKCC1-transfected HEK293 cells. Data represents mean±sem from 5 independent experiments. c) Quantification of the effect of bumetanide and furosemide and 2 selected compounds (3.8, 3.17) in the chloride kinetic assay on NKCC1-transfected HEK293 cells. Data represents mean±sem from 5 independent experiments, and they are represented as % of the controls. *P<0.05, **P<0.01, ***P<0.001 Kruskal-Wallis Anova (Dunn's Post hoc Test); ###P<0.001 two-tailed unpaired Student t-test.

FIG. 2: In vitro testing of the NKCC1 inhibitors in a calcium kinetic assay. a) Example traces of fluorescence levels upon application of GABA (100 μM) and KCl (90 mM) used to trigger calcium influx in primary neuronal cultures treated after 3 days in culture (3 DIV) with vehicle, bumetanide, furosemide and compounds 3.8, 3.13 and 3.17 in the calcium kinetic assay. b) Quantification of the average fluorescence increase upon GABA application normalized to the increase upon KCl application in neurons treated with bumetanide, furosemide and 3 exemplary compounds (3.8, 3.13, 3.17) (10 μM, 100 μM). Data represents mean±sem from 5 independent experiments, and they are presented as % of the control. *P<0.05, **P<0.01, ***P<0.001 Kruskal-Wallis Anova (Dunn's Post hoc Test).

FIG. 3: Assessment of drug-likeness of a selected compound, compound 3.17. a) Chemicophysical properties of bumetanide and compound 3.17 by LC-MS analysis. b) Comparison between urinary volume of WT mice (C57Bl/6N) two hours after treatment with bumetanide (0.2 mg/kg) and compound 3.17 (0.2 mg/kg). c) Assessment of urinary volume of Ts65Dn mouse model of Down syndrome and WT littermates two hours after treatment with compound 3.17 (0.2 mg/kg). Number in parenthesis: number of analyzed animals. Data represents mean±sem, and they are presented as % of the respective vehicle.

FIG. 4: In vivo assessment of the efficacy of the selected NKCC1 inhibitor in Ts65Dn mice. (a) Quantification of the discrimination index in mice treated with vehicle (WT, n=14, Ts65Dn, n=10;) or 3.17 (WT, n=14, Ts65Dn, n=11;) ***P<0.001; two-way ANOVA Tukey's post hoc test. (b) Quantification of the discrimination index in mice treated with vehicle (WT, n=14, Ts65Dn, n=10;) or 3.17 (WT, n=14, Ts65Dn, n=11) *P<0.05, **P<0.01 two-way ANOVA Tukey's post hoc test. (c) Quantification of the correct choices in mice treated with vehicle (WT, n=14, Ts65Dn, n=10;) or 3.17 (WT, n=14, Ts65Dn, n=11;) ***P<0.001; two-way ANOVA Tukey's post hoc test. (d) Quantification of the freezing response in mice treated with vehicle (WT, n=14, Ts65Dn, n=10;) or 3.17 (WT, n=14, Ts65Dn, n=11;) *P<0.05, **P<0.01 two-way ANOVA Tukey's post hoc test.

FIGS. 5 to 16: reports the synthetic procedures schemes 1 to 15 for preparing the compounds of the invention.

FIG. 17: shows the results of the in vitro testing of the selective NKCC1 inhibitors in the thallium-based assay on NKCC2 transfected HEK cells.

FIGS. 18a-18d: shows the results of the in vivo assessment of the efficacy of the compound 3.17 in VPA-induced mouse model of autism; (a) Left panel, quantification of the sociability index in mice treated with vehicle (WT, n=15, VPA, n=10) or 3.17 (WT, n=9, VPA, n=12); two-way ANOVA on Ranks, Tukey's post hoc test, **P<0.01. Right panel, quantification of the social novelty index in mice treated with vehicle (WT, n=15, VPA, n=10) or 3.17 (WT, n=9, VPA, n=12); two-way ANOVA, Tukey's post hoc test, *P<0.05, **P<0.01. (b) Quantification of the interaction time in mice treated with vehicle (WT, n=15, VPA, n=10) or 3.17 (WT, n=10, VPA, n=11); two-way ANOVA, Tukey's post hoc test, **P<0.01. (c) Quantification of the number of marbles buried by mice treated with vehicle (WT, n=17, VPA, n=17) or 3.17 (WT, n=13, VPA, n=13); two-way ANOVA, Tukey's post hoc test, *P<0.05, **P<0.01. (d) Quantification of grooming time for mice treated with vehicle (WT, n=20, VPA, n=17) or 3.17 (WT, n=13, VPA, n=13); two-way ANOVA on Ranks, Tukey's post hoc test, *P<0.05, **P<0.01.

### DETAILED DESCRIPTION OF THE INVENTION

The present invention provides 2-aminobenzenesulfonamides derivatives, according to Formula Ia, Ib and Ic, which are NKCC1 inhibitors and solve the need for alternative compounds to bumetanide and, particularly, compounds capable of restoring the GABA A signaling through NKCC1 inhibition.

In one aspect, the invention provides a compound having Formula Ia or a pharmaceutically acceptable salt thereof or stereoisomeric forms thereof, or the individual geometrical isomers, enantiomers, diastereoisomers, tautomers, zwitterions and pharmaceutically acceptable salts thereof:

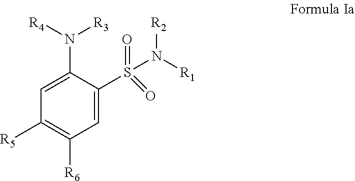

wherein:

R₁ and R₂ are independently
hydrogen;
linear or branched, C₁₋₁₀ alkyl optionally comprising one or more unsaturations and optionally substituted by groups selected from the group consisting of halogens, —OH, —C₃₋₈cycloalkyl, non-aromatic heterocycles, aromatic heterocycles, —C₁₋₆alkoxyalkyl, —NH₂, —NO₂, amides, carboxylic acids, ketones, ethers, esters, aldehydes, or sulfonamides;
linear or branched substituted or unsubstituted C₃₋₈ cycloalkyl;
linear or branched substituted or unsubstituted C₄₋₁₀ cycloalkylalkyl;
C₃₋₈ heterocycloalkyl;
optionally substituted phenyl;
or R₁ and R₂, together with the nitrogen atom to which they are attached, form a substituted or unsubstituted saturated heterocycle;

R₃ and R₄ are independently
hydrogen;
linear or branched C₁₋₁₀ alkyl optionally comprising one or more unsaturations and optionally substituted by groups selected from the group consisting of halogens, —OH, —C₃₋₈cycloalkyl, non-aromatic heterocycles, aromatic heterocycles, —C₁₋₆alkoxyalkyl, —NH₂, —NO₂, amides, carboxylic acids, ketones, ethers, esters, aldehydes, or sulfonamides;
C₃₋₁₀ cycloalkyl;
C₄₋₁₀ cycloalkylalkyl;
C₂₋₈ haloalkyl;
linear or branched C₂₋₈ heteroalkyl, substituted or unsubstituted;
optionally substituted phenyl;

provided that at least one of R₃ and R₄ is other than hydrogen;
or R₃ and R₄, when taken together with the nitrogen atom to which they are attached, form a substituted or unsubstituted saturated heterocycle;

R₅ is
hydrogen;
halogen;
hydroxyl;
—O—C₁₋₁₀alkyl;
—O—C₃₋₁₀cycloalkyl;
—O—C₃₋₈heterocycloalkyl;
C₁₋₁₀ alkoxyalkyl;
C₃₋₁₀ alkoxycycloalkyl;
optionally substituted phenoxyl;
—NH₂;
C₁₋₈ alkylamine;
C_{2-C16} dialkylamine;
Aniline;
—SH;
C₁₋₈ alkylthioether;
thiophenol;
—NO₂;

R₆ is
nitro;
nitrile;
—CH₂OH;
carboxylic acid;
C₁₋₄ alkyl ester;
C₂₋₈ heteroalkyl ester;
C₃₋₆ cycloalkyl ester;
phenyl ester;
carboxamide;
cyclic amide;
tetrazole;

provided that when R₆ is nitro, the following conditions are satisfied at the same time:
R₁ is other than H,
R₂ is other than linear or branched, unsubstituted C₂₋₆ alkyl,
R₃ is other than H,
R₄ is other than linear, unsubstituted C₁₋₃ alkyl,
R₅ is other than H;

and provided that the compound of formula Ia is not one of the following:

| R₁ | R₂ | R₃ | R₄ | R₅ | R₆ |
|---|---|---|---|---|---|
| H | (CH₂)₃—OCH₃ | H | CH₂CH₃ | H | —NO₂ |
| H | CH₂CH(CH₃)—OCH₃ | H | CH₂CH₃ | H | —NO₂ |
| H | (CH₂)₂—OCH₃ | H | CH₂CH₃ | H | —NO₂ |
| H | (CH₂)₂—OCH₃ | H | CH₂CH₂CH₃ | H | —NO₂ |
| H | CH(CH₃)CH₂—OCH₃ | H | CH₂CH₃ | H | —NO₂ |
| H | CH₃ | H | CH₂CH₂OH | H | COOH |
| H | CH₃ | H | CH₂CH₃ | H | COOH |
| H | CH₃ | H | CH₂CH₂CH₂CH₃ | H | COOH |
| H | phenyl | H | cyclohexyl | H | COOH |

In one embodiment:

R₁ and R₂ are independently
hydrogen;
linear or branched, C₁₋₁₀ alkyl optionally comprising one or more unsaturations and optionally substituted by groups selected from the group consisting of halogens, —OH, —C₃₋₈cycloalkyl, non-aromatic heterocycles, aromatic heterocycles, —C₁₋₆alkoxyalkyl, —NH₂, —NO₂, amides, carboxylic acids, ketones, ethers, esters, aldehydes, or sulfonamides;
linear or branched substituted or unsubstituted C₃₋₈ cycloalkyl;
linear or branched substituted or unsubstituted C₄₋₁₀ cycloalkylalkyl;
optionally substituted phenyl;
or R₁ and R₂, together with the nitrogen atom to which they are attached, form a substituted or unsubstituted saturated heterocycle;

R₃ and R₄ are independently
hydrogen;
linear or branched C₁₋₁₀ alkyl optionally comprising one or more unsaturations and optionally substituted by groups selected from the group consisting of halogens, —OH, —C₃₋₈cycloalkyl, non-aromatic heterocycles, aromatic heterocycles, —C₁₋₆alkoxyalkyl, —NH₂, —NO₂, amides, carboxylic acids, ketones, ethers, esters, aldehydes, or sulfonamides;
C₃₋₁₀ cycloalkyl;
C₄₋₁₀ cycloalkylalkyl;
C₂₋₈ haloalkyl;
linear or branched C₂₋₈ heteroalkyl, substituted or unsubstituted;
optionally substituted phenyl;

provided that at least one of R₃ and R₄ is other than hydrogen;
or R₃ and R₄, when taken together with the nitrogen atom to which they are attached, form a substituted or unsubstituted saturated heterocycle;

R₅ is
hydrogen;
halogen;
hydroxyl;
C₁₋₁₀ alkoxyalkyl;
C₃₋₁₀ alkoxycycloalkyl;
optionally substituted phenoxyl;
—NH₂;
C₁₋₈ alkylamine;
C_{2-C16} dialkylamine;
aniline;
—SH;
C₁₋₈ alkylthioether;
thiophenol;
—NO₂;

R₆ is
nitro;
nitrile;
—CH₂OH;
carboxylic acid;
C₁₋₄ alkyl ester;
C₂₋₈ heteroalkyl ester;
C₃₋₆ cycloalkyl ester;
phenyl ester;
carboxamide;
cyclic amide;
tetrazole.

In a preferred embodiment, R₁ and R₂ are independently H, —CH₃, cyclopentane, cyclohexane, 4-tetrahydropyran, or, together with the nitrogen atom to which they are attached are a morpholine, a piperidine optionally substituted with at least one halogen, a pirrolidine.

Still more preferably, R₁ and R₂ are independently —CH₃, —C₂H₅, —C₃H₇, —C₄H₉. In a preferred embodiment, R₁ and R₂ are both —CH₃.

In a preferred embodiment, R₃ and R₄ are independently hydrogen, linear or branched —C₁₋₈alkyl optionally substituted with at least one C₁₋₆ alkoxyalkyl, —C₂₋₈haloalkyl, or R₃ and R₄, when taken together with the nitrogen atom to which they are attached, are a substituted or unsubstituted saturated heterocycle.

Still more preferably, R₃ and R₄ are independently H, —C₄H₉, —C₆H₁₃, —C₈H₁₇, —C₂H₄C(CH₃)₃, —C₇H₁₄CF₃, —C₃H₆CF₃, —C₅H₁₀CF₃, —C₂H₄OCH₃, —C₄H₈OCH₃, —C₆H₁₂OCH₃, or, together with the nitrogen atom to which they are attached, are a piperazine, preferably a substituted piperazine, still more preferably a —N(C₄H₈CF₃)piperazine.

Still more preferably, R₃ and R₄ are independently —CH₃, —C₂H₅, —₃H₇, —C₄H₉, —C₅H₁₁, —C₆H₁₃, —C₇H₁₅, —C₈H₁₇ or —C₁₋₈ haloakyl. In a preferred embodiment, R₃ is H and R₄ is C₇H₁₄CF₃.

For the purposes of the present invention, one or more of the hydrogen atoms of the above detailed compounds may be substituted with deuterium.

In a preferred embodiment, R₅ is hydrogen, halogen or hydroxyl, more preferably is hydrogen.

In a preferred embodiment, R₆ is carboxylic acid, C₁₋₄ alkyl ester, nitro or nitrile, more preferably is carboxylic acid.

In an embodiment, the claimed compound is compound 3.17, having the formula here below reported.

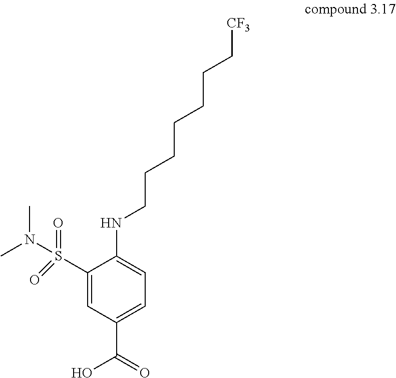 [SMILES: CN(C)S(=O)(=O)c1cc(C(=O)O)ccc1NCCCCCCCC(F)(F)F]

### Definitions

Unless otherwise specified in the present description, it should be understood that the terms used herein have the following meanings.

The term “alkyl”, as used herein, as sole substitutent or as part of a larger substituent, refers to saturated, monovalent or divalent hydrocarbon moieties having linear or branched moieties or combinations thereof and containing 1 to 10, preferably 1 to 8 carbon atoms and still more preferably 1 to 4 carbon atoms. Suitable examples include methyl, ethyl, n-propyl, iso-propyl, n-butyl, iso-butyl, sec-butyl, tert-butyl, n-pentyl, iso-pentyl, 2-methylbutyl, neo-pentyl, 1-ethylpropyl, n-hexyl, iso-hexyl, 4-methylpentyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl, 2-ethylbutyl, 1-methyl-2-methylpropyl and the like. Hydrogen atoms on alkyl groups can be substituted by groups including, but not limited to: deuterium, halogens, —OH, —C₃₋₈cycloalkyl, non-aromatic heterocycles, aromatic heterocycles,

—C₁₋₆ alkoxyalkyl, —NH₂, —NO₂, amides, carboxylic acids, ketones, ethers, esters, aldehydes, or sulfonamides.

For the purposes of the present invention, the alkyl substituent may comprise one or more unsaturations.

The term “cycloalkyl”, as used herein, refers to a monovalent or divalent ring of 3 to 10 carbon atoms, or 3 to 8 carbon atoms derived from a saturated cyclic hydrocarbon. Cycloalkyl groups can be monocyclic or polycyclic. Cycloalkyl can be substituted by groups including, but not limited to: halogens, —OH, —C₃₋₈ cycloalkyl, non-aromatic heterocycles, aromatic heterocycles, —C₁₋₆alkoxyalkyl, —NH₂, —NO₂, amides, ethers, esters, carboxylic acids, aldehydes, ketones, sulfonamides groups.

Examples of the cycloalkylalkyl groups include a cyclobutylethyl group, a cyclobutylpropyl group, a cyclopentylmethyl group, a cyclopentylethyl group, a cyclopentylpropyl group, a cyclohexylmethyl group, a cyclohexylethyl group, a cyclohexylpropyl group, a cycloheptylmethyl group and a cycloheptylethyl group.

The term “haloalkyl” as used herein refers to an alkyl group partially or fully substituted with halogen atoms which may be the same or different. Examples of “haloalkyl” include —CH₂CF₃ and —CCl₂CF₃.

In the present invention, “alkoxy” includes, for example, the aforementioned alkyl-O— group and, for example, methoxy, ethoxy, n-propoxy, isopropoxy, n-butoxy and the like can be mentioned, and “alkoxyalkyl” is, for example, methoxymethyl or the like, and “aminoalkyl” is, for example, 2-aminoethyl or the like. In the present invention, “halogen” refers to any halogen element, which is, for example, fluorine, chlorine, bromine or iodine.

The term “heterocycle” as used herein, refers to a 3 to 8 membered ring, which can be aromatic or non-aromatic, containing at least one heteroatom selected from O or N or S or combinations of at least two of them, interrupting the carbocyclic ring structure. The heterocyclic ring can comprise a C═O; the S heteroatom can be oxidized. Heterocycles can be monocyclic or polycyclic. Heterocyclic ring moieties can be substituted by groups including, but not limited to: halogens, —OH, —C₃₋₈cycloalkyl, non-aromatic heterocycles, aromatic heterocycles, —C₁₋₆alkoxyalkyl, —NH₂, —NO₂, amides, ethers, esters, aldehydes, carboxylic acids, ketones, sulfonamides groups. Preferred imidazoline, pyrazoline, pyperidine, pyperazine, morpholine, thiomorpholine, azepane, azocane.

The term “substituted heterocycle”, as used herein, refers to heterocycles optionally substituted with halogens, —C₁₋₅ alkyl, —C₁₋₅ alkenyl, —C₁₋₅ haloalkyl.

The term “alkenyl”, as used herein, refers to a monovalent or divalent hydrocarbon radical having 2 to 6 carbon atoms, derived from a saturated alkyl, having at least one double bond. —C₂₋₆ alkenyl can be in the E or Z configuration. Alkenyl groups can be substituted by —C₁₋₆ alkyl.

The terms “substituted phenyl” or “substituted phenoxyl”, as used herein, refer to a phenyl radical substituted with a substituent selected from the group consisting of C₁₋₈alkyl, preferably methyl, C₁₋₈alkoxy, preferably methoxy, hydroxyl, trifluoromethyl, nitro, amine, halogen.

The term “pharmaceutically acceptable salts” refers to salts or complexes that retain the desired biological activity of the above identified compounds and exhibit minimal or no undesired toxicological effects. The “pharmaceutically acceptable salts” according to the invention include therapeutically active, non-toxic base or acid salt forms, which the compounds of Formula I are able to form.

Compounds of Formula Ia and their salts can be in the form of a solvate, which is included within the scope of the present invention. Such solvates include for example hydrates, alcoholates and the like.

With respect to the present invention reference to a compound or compounds is intended to encompass that compound in each of its possible isomeric forms and mixtures thereof unless the particular isomeric form is referred to specifically.

Compounds according to the present invention may exist in different polymorphic forms; although not explicitly indicated in the above formula, such forms are intended to be encompassed within the scope of the present invention.

In an embodiment, compounds of formula Ia are selected from the group consisting of:
1.6 2-(butylamino)-5-nitro-benzenesulfonamide,
1.7 2-(hexylamino)-5-nitro-benzenesulfonamide,
1.8 5-nitro-2-(octylamino)benzenesulfonamide,
1.9 2-(3,3-dimethylbutylamino)-5-nitro-benzenesulfonamide,
1.10 2-(butylamino)-N-methyl-5-nitro-benzenesulfonamide,
1.11 2-(hexylamino)-N-methyl-5-nitro-benzenesulfonamide,
1.12 N-methyl-5-nitro-2-(octylamino)benzenesulfonamide,
1.13 2-(3,3-dimethylbutylamino)-N-methyl-5-nitro-benzenesulfonamide,
1.14 2-(butylamino)-N,N-dimethyl-5-nitro-benzenesulfonamide,
1.15 2-(hexylamino)-N,N-dimethyl-5-nitro-benzenesulfonamide,
1.16 N,N-dimethyl-5-nitro-2-(octylamino)benzenesulfonamide,
1.17 2-(3,3-dimethylbutylamino)-N,N-dimethyl-5-nitro-benzenesulfonamide,
2.2 4-(butylamino)-2-chloro-5-sulfamoyl-benzoic acid,
2.3 2-chloro-4-(hexylamino)-5-sulfamoyl-benzoic acid,
2.4 2-chloro-4-(octylamino)-5-sulfamoyl-benzoic acid,
2.5 2-chloro-4-(3,3-dimethylbutylamino)-5-sulfamoyl-benzoic acid,
2.6 4-(butylamino)-3-sulfamoyl-benzoic acid,
2.7 4-(hexylamino)-3-sulfamoyl-benzoic acid,
2.8 4-(octylamino)-3-sulfamoyl-benzoic acid,
2.9 4-(3,3-dimethylbutylamino)-3-sulfamoyl-benzoic acid,
3.6 4-(butylamino)-3-(methylsulfamoyl)benzoic acid,
3.7 4-(hexylamino)-3-(methylsulfamoyl)benzoic acid,
3.8 3-(methylsulfamoyl)-4-(octylamino)benzoic acid,
3.9 4-(3,3-dimethylbutylamino)-3-(methylsulfamoyl)benzoic acid,
3.10 3-(methylsulfamoyl)-4-(8,8,8-trifluorooctylamino)benzoic acid,
3.11 4-(butylamino)-3-(dimethylsulfamoyl)benzoic acid,
3.12 3-(dimethylsulfamoyl)-4-(hexylamino)benzoic acid,
3.13 3-(dimethylsulfamoyl)-4-(octylamino)benzoic acid,
3.14 4-(3,3-dimethylbutylamino)-3-(dimethylsulfamoyl)benzoic acid,
3.15 3-(dimethylsulfamoyl)-4-(4,4,4 trifluorobutylamino) benzoic acid,
3.16 3-(dimethylsulfamoyl)-4-(6,6,6-trifluorohexylamino) benzoic acid,
3.17 3-(dimethylsulfamoyl)-4-(8,8,8-trifluorooctylamino) benzoic acid,
3.18 3-(dimethylsulfamoyl)-4-(2-methoxyethylamino)benzoic acid,
3.19 3-(dimethylsulfamoyl)-4-(4-methoxybutylamino)benzoic acid,
3.20 3-(dimethylsulfamoyl)-4-(6-methoxyhexylamino)benzoic acid,
3.21 3-(cyclopentylsulfamoyl)-4-(8,8,8-trifluorooctylamino) benzoic acid,
3.22 3-(cyclohexylsulfamoyl)-4-(8,8,8-trifluorooctylamino) benzoic acid,
5.5 3-pyrrolidin-1-ylsulfonyl-4-(8,8,8-trifluorooctylamino) benzoic acid,
5.6 3-(1-piperidylsulfonyl)-4-(8,8,8-trifluorooctylamino) benzoic acid,
5.7 3-morpholinosulfonyl-4-(8,8,8-trifluorooctylamino) benzoic acid,
6.3 5-cyano-N,N-dimethyl-2-(8,8,8-trifluorooctylamino) benzenesulfonamide,
7.4 2-hydroxy-5-sulfamoyl-4-(8,8,8-trifluorooctylamino) benzoic acid,
9.1 3-(dimethylsulfamoyl)-4-[4-(5,5,5 trifluoropentyl)piperazin-1-yl] benzoic acid,
10.1 N,N-dimethyl-5-(1H-tetrazol-5-yl)-2 (8,8,8-trifluorooctylamino)benzenesulfonamide,
12.3 Methyl 5-(N,N-dimethylsulfamoyl)-2-methoxy-4-((8,8,8-trifluorooctyl)amino)benzoate,
12.4 Methyl 5-(N,N-dimethylsulfamoyl)-2-hydroxy-4-((8,8,8-trifluorooctyl)amino)benzoate,
12.5 Methyl 5-(N,N-dimethylsulfamoyl)-2-ethoxy-4-((8,8,8-trifluorooctyl)amino)benzoate
12.6 Methyl 2-(cyclopentyloxy)-5-(N,N-dimethylsulfamoyl)-4-((8,8,8-trifluorooctyl)amino)benzoate,
12.7 5-(N,N-dimethylsulfamoyl)-2-ethoxy-4-((8,8,8-trifluorooctyl)amino)benzoic acid,
12.8 2-(cyclopentyloxy)-5-(N,N-dimethylsulfamoyl)-4-((8,8,8-trifluorooctyl)amino)benzoic acid,
13.1 5-(N,N-dimethylsulfamoyl)-2-methoxy-4-((8,8,8-trifluorooctyl)amino)benzoic acid,
14.3 3-morpholinosulfonyl-4-((8,8,8-trifluorooctyl)amino)benzoic acid
14.4 3-((4,4-difluoropiperidin-1-yl)sulfonyl)-4-((8,8,8-trifluorooctyl)amino)benzoic acid,
15.1 3-(dimethylsulfamoyl)-4-(kept-6-enylamino)benzoic acid,
15.2 Methyl 3-(N,N-dimethylsulfamoyl)-4-(hept-6-en-1-ylamino)benzoate,
15.3 Methyl 4-((8-bromo-8,8-difluorooctyl)amino)-3-(N,N-dimethylsulfamoyl)benzoate,
15.4 4-[(8-bromo-8,8-difluoro-octyl)amino]-3-(dimethylsulfamoyl)benzoic acid,
16.1 5-(dimethylsulfamoyl)-2-isopropoxy-4-(8,8,8-trifluorooctylamino)benzoic acid,
16.2 2-(cyclohexoxy)-5-(dimethylsulfamoyl)-4-(8,8,8-trifluorooctylamino)benzoic acid,
16.3 5-(dimethylsulfamoyl)-2-tetrahydropyran-4-yloxy-4-(8,8,8-trifluorooctylamino)benzoic acid,
16.4 2-(cyclobutoxy)-5-(dimethylsulfamoyl)-4-(8,8,8-trifluorooctylamino)benzoic acid,
16.5 5-(dimethylsulfamoyl)-2-(oxetan-3-yloxy)-4-(8,8,8-trifluorooctylamino)benzoic acid,
16.6 5-(dimethylsulfamoyl)-2-(4-piperidyloxy)-4-(8,8,8-trifluorooctylamino)benzoic acid,
16.7 5-(dimethylsulfamoyl)-2-phenoxy-4-(8, 8,8-trifluorooctylamino)benzoic acid.

Preferably, compounds of formula Ia are selected from the group consisting of:
1.7 2-(hexylamino)-5-nitro-benzenesulfonamide,
1.17 2-(3,3-dimethylbutylamino)-N,N-dimethyl-5-nitro-benzenesulfonamide,
2.2 4-(butylamino)-2-chloro-5-sulfamoyl-benzoic acid,
2.6 4-(butylamino)-3-sulfamoyl-benzoic acid,
2.7 4-(hexylamino)-3-sulfamoyl-benzoic,
2.8 4-(octylamino)-3-sulfamoyl-benzoic acid,
2.9 4-(3,3-dimethylbutylamino)-3-sulfamoyl-benzoic acid,
3.6 4-(butylamino)-3-(methylsulfamoyl)benzoic acid,
3.7 4-(hexylamino)-3-(methylsulfamoyl)benzoic acid,
3.8 3-(methylsulfamoyl)-4-(octylamino)benzoic acid,
3.9 4-(3,3-dimethylbutylamino)-3-(methylsulfamoyl)benzoic acid,
3.10 3-(methylsulfamoyl)-4-(8,8,8-trifluorooctylamino)benzoic acid,
3.11 4-(butylamino)-3-(dimethylsulfamoyl)benzoic acid,
3.12 3-(dimethylsulfamoyl)-4-(exylamino)benzoic acid,
3.13 3-(dimethylsulfamoyl)-4-(octylamino)benzoic acid,
3.14 4-(3,3-dimethylbutylamino)-3-(dimethylsulfamoyl)benzoic acid,
3.17 3-(dimethylsulfamoyl)-4-(8,8,8-trifluorooctylamino) benzoic acid,
3.20 3-(dimethylsulfamoyl)-4-(6-methoxyhexylamino)benzoic acid,
3.21 3-(cyclopentylsulfamoyl)-4-(8,8,8-trifluorooctylamino) benzoic acid,
3.22 3-(cyclohexylsulfamoyl)-4-(8,8,8-trifluorooctylamino) benzoic acid,
5.5 3-pyrrolidin-1-ylsulfonyl-4-(8,8,8-trifluorooctylamino) benzoic acid,
5.6 3-(1-piperidylsulfonyl)-4-(8,8,8-trifluorooctylamino) benzoic acid,
5.7 3-morpholinosulfonyl-4-(8,8,8-trifluorooctylamino) benzoic acid
13.1 5-(N,N-dimethylsulfamoyl)-2-methoxy-4-((8,8,8-trifluorooctyl)amino)benzoic acid,
14.4 3-((4,4-difluoropiperidin-1-yl)sulfonyl)-4-((8,8,8-trifluorooctyl)amino)benzoic acid,
15.1 3-(dimethylsulfamoyl)-4-(kept-6-enylamino)benzoic acid.

In a further embodiment, compounds of formula Ia are selected from the group consisting of:
1.7 2-(hexylamino)-5-nitro-benzenesulfonamide,
1.15 2-(hexylamino)-N,N-dimethyl-5-nitro-benzenesulfonamide,
2.2 4-(butylamino)-2-chloro-5-sulfamoyl-benzoic acid,
2.6 4-(butylamino)-3-sulfamoyl-benzoic acid,
2.7 4-(hexylamino)-3-sulfamoyl-benzoic acid,
2.8 4-(octylamino)-3-sulfamoyl-benzoic acid,
3.8 3-(methylsulfamoyl)-4-(octylamino)benzoic acid,
3.13 3-(dimethylsulfamoyl)-4-(octylamino)benzoic acid,
3.14 4-(3,3-dimethylbutylamino)-3-(dimethylsulfamoyl)benzoic acid, and
3.17 3-(dimethylsulfamoyl)-4-(8,8,8-trifluorooctylamino) benzoic acid.

According a second aspect of the invention it is provided a compound of formula Ib or a pharmaceutically acceptable salt thereof or stereoisomeric forms thereof, or the individual geometrical isomers, enantiomers, diastereoisomers, tautomers, zwitterions and pharmaceutically acceptable salts thereof:

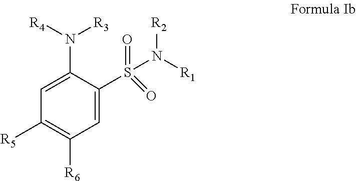

wherein:

R₁ and R₂ are independently
hydrogen;
linear or branched, unsubstituted or substituted C₁₋₁₀ alkyl optionally comprising one or more unsaturations;
linear or branched substituted or unsubstituted C₃₋₈ cycloalkyl;
linear or branched substituted or unsubstituted C₄₋₁₀ cycloalkylalkyl;
C₃₋₈ heterocycloalkyl;
optionally substituted phenyl;
or R₁ and R₂, together with the nitrogen atom to which they are attached, form a substituted or unsubstituted saturated heterocycle;

R₃ and R₄ are independently
hydrogen;
unsubstituted or substituted C₁₋₁₀ alkyl optionally comprising one or more unsaturations;
C₃₋₁₀ cycloalkyl;
C₄₋₁₀ cycloalkylalkyl;
C₂₋₈ haloalkyl;
linear or branched C₂₋₈ heteroalkyl, substituted or unsubstituted;
optionally substituted phenyl;

provided that at least one of R₃ and R₄ is other than hydrogen;
or R₃ and R₄, when taken together with the nitrogen atom to which they are attached, form a substituted or unsubstituted saturated heterocycle;

R₅ is
hydrogen;
halogen;
hydroxyl;
—O—C₁₋₁₀alkyl;
—O—C₃₋₁₀cycloalkyl;
—O—C₃₋₈heterocycloalkyl;
C₁₋₁₀ alkoxyalkyl;
C₃₋₁₀ alkoxycycloalkyl;
optionally substituted phenoxyl;
—NH₂;
C₁₋₈ alkylamine;
C_{2-C16} dialkylamine;
aniline;
—SH;
C₁₋₈ alkylthioether;
thiophenol;
—NO₂;

R₆ is
nitro;
nitrile;
—CH₂OH;
carboxylic acid;
C₁₋₄ alkyl ester;
C₂₋₈ heteroalkyl ester;
C₃₋₆ cycloalkyl ester;
phenyl ester;
carboxamide;
C₁₋₄ alkylamide;
C₂₋₈ dialkylamide;
cycloalkyl amide;
cyclic amide;
tetrazole;

for the use as a medicament.

In a further embodiment, it is provided a compound of formula Ic or a pharmaceutically acceptable salt thereof or stereoisomeric forms thereof, or the individual geometrical isomers, enantiomers, diastereoisomers, tautomers, zwitterions and pharmaceutically acceptable salts thereof:

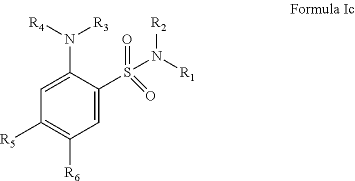

wherein:

R₁ and R₂ are independently
hydrogen;
linear or branched, unsubstituted or substituted C₁₋₁₀ alkyl optionally comprising one or more unsaturations;
linear or branched substituted or unsubstituted C₃₋₈ cycloalkyl;
linear or branched substituted or unsubstituted C₄₋₁₀ cycloalkylalkyl;
optionally substituted phenyl;
or R₁ and R₂, together with the nitrogen atom to which they are attached, form a substituted or unsubstituted saturated heterocycle;

R₃ and R₄ are independently
hydrogen;
substituted or unsubstituted C₁₋₁₀ alkyl optionally comprising one or more unsaturations;
C₃₋₁₀ cycloalkyl;
C₄₋₁₀ cycloalkylalkyl;
C₂₋₈ haloalkyl;
linear or branched C₂₋₈ heteroalkyl, substituted or unsubstituted;
optionally substituted phenyl;

provided that at least one of R₃ and R₄ is other than hydrogen;
or R₃ and R₄, when taken together with the nitrogen atom to which they are attached, form a substituted or unsubstituted saturated heterocycle;

R₅ is
hydrogen;
halogen;
hydroxyl;
C₁₋₁₀ alkoxyalkyl;
C₃₋₁₀ alkoxycycloalkyl;
optionally substituted phenoxyl;
—NH₂;
C₁₋₈ alkylamine;
C_{2-C16} dialkylamine;
aniline;
—SH;
C₁₋₈ alkylthioether;
thiophenol;
—NO₂;

R₆ is
nitro;
nitrile;
—CH₂OH;
carboxylic acid;
C₁₋₄ alkyl ester;
C₂₋₈ heteroalkyl ester;
C₃₋₆ cycloalkyl ester;
phenyl ester;
carboxamide;
C₁₋₄ alkylamide;
C₂₋₈ dialkylamide;
cycloalkyl amide;
cyclic amide;
tetrazole;

for the use as a medicament.

The compounds of formulae Ib and Ic are indicated for use in treating or preventing conditions in which there is likely to be a component associated to depolarizing GABAergic transmission due to increased NKCC1 or decreased KCC2 expression levels or function.

In an embodiment of the invention, there are provided pharmaceutical compositions including at least one compound of formulae Ib or Ic in a pharmaceutically acceptable carrier.

In a further embodiment, there are provided methods for treating disorders associated to depolarizing GABAergic transmission due to increased NKCC1 or decreased KCC2 expression levels or function; such methods can be performed, for example, by administering to a subject in need thereof a pharmaceutical composition containing a therapeutically effective amount of at least one compound of formulae Ib or Ic.

Advantageously, said method has shown not to have the diuretic side-effect.

These compounds are useful for the treatment of mammals, including humans.

The actual amount of the compound to be administered in any given case will be determined by a physician taking into account the relevant circumstances, such as the severity of the condition, the age and weight of the patient, the patient's general physical condition, the cause of the condition, and the route of administration. Additionally, the formulations may be designed to provide a sustained release of the active compound over a given period of time, or to carefully control the amount of drug released at a given time during the course of therapy.

In view of the chemical structure of the compounds of the invention, a suitable formulation can be prepared to allow an effective amount of the drug to pass the blood brain barrier; as an example nanoformulations may be prepared.

Since individual subjects may present a wide variation in severity of symptoms and each drug has its unique therapeutic characteristics, the precise mode of administration and dosage employed for each subject is left to the discretion of the practitioner.

The 2-aminobenzenesulfonamide derivatives have been demonstrated to be potent inhibitors towards the NKCC1 transporter, displaying good inhibition percentage at 10 micromolar and 100 micromolar concentration in cell-based assays. In addition, the compounds have shown a remarkable activity in Down syndrome mouse models (Ts65Dn mice), rescuing hippocampus-dependent cognitive behaviors at a 0.2 mg/kg dosing. Notably, the treatment in vivo with these compounds had no statistically significant diuretic effect at 0.2 mg/kg when compared to vehicle-treated animals in C57B16N mice, Ts65Dn mice and their wild time littermates. Further, the compounds have shown a remarkable efficacy in restoring sociability in a rodent model of drug-induced autism.

In a second aspect, the present invention relates to the compounds of formula Ib or Ic for use in the treatment of diseases or disorders associated to depolarizing GABAergic transmission due to increased NKCC1 or decreased KCC2 (relative to physiological or desired) levels of expression or function. In particular, the compounds here described are for use in the treatment of Down syndrome, neuropathic pain, stroke, cerebral ischemia, cerebral edema, hydrocephalous, traumatic brain injury, Brain Trauma-Induced Depressive-Like Behavior, autism spectrum disorders (i.e. autism, Fragile X, Rett, Asperger and DiGeorge syndromes) epilepsy, seizures, epileptic state, West syndrome, glioma, glioblastoma, anaplastic astrocytoma, Parkinson's disease, Hungtinton's disease, schizophrenia, anxiety, Tuberous Sclerosis Complex and associated behavioural problems, Dravet syndrome.

The invention could be useful either as a stand-alone therapeutic, or in combination with other psychoactive drugs including but not limited to Fluoxetine, Memantine, Donepezil, DAPT, anti-inflammatory drugs including but not limited to acetaminophen and other COX inhibitors, anti-oxidants and psychoactive food supplements including but not limited to melatonin, EGCG, resveratrol, omega-3, folinic acid, selenium, zinc, vitamin A, E and C. In addition, the invention could be useful in combination with early educational therapies.

The compounds here described are, in a preferred embodiment, characterized by an amino substituent in orto position of the benzenesulfonamide scaffold, a carboxylic acid substituent in meta position of the benzenesulfonamide scaffold, the presence of an amino group with at least one substituent different from hydrogen, the absence of aromatic substituents on the benzenesulfonamide scaffold.

Surprisingly, the compounds here described showed an efficient inhibition of NKCC1 when compared to bumetanide.

As a further advantage, the compounds of the invention has shown a particular NKCC1/NKCC2 selectivity, thus making them highly desirable.

Also, the compounds of the invention are characterized by having no diuretic effect.

In a still further advantage, the compounds of the invention have shown a NKCC1/NKCC2 selectivity, which is not accompanied by a diuretic effect.

In particular, compound 3.17 of the invention as below disclosed has shown the highest NKCC1/NKCC2 selectivity.

### EXAMPLES

### Example 1: Chemical Synthesis and Characterization

All the commercial available reagents and solvents were used as purchased from vendors without further purification. Dry solvents were purchased from Sigma-Aldrich. Automated column chromatography purifications were done using a Teledyne ISCO apparatus (CombiFlash® Rf) with pre-packed silica gel or basic alumina columns of different sizes (from 4 g up to 120 g) and mixtures of increasing polarity of cyclohexane and ethyl acetate (EtOAc), cyclohexane and tert-ButylMethyl eter (TBME) or dicloromethane (DCM) and methanol (MeOH). NMR experiments were run on a Bruker Avance III 400 system (400.13 MHz for 1H, and 100.62 MHz for 13C), equipped with a BBI probe and Z-gradients. Spectra were acquired at 300 K, using deuterated dimethylsulfoxide (DMSO-d6) or deuterated chloroform (CDCl3) as solvents. For 1H-NMR, data are reported as follows: chemical shift, multiplicity (s=singlet, d=doublet, dd=double of doublets, t=triplet, q=quartet, m=multiplet), coupling constants (Hz) and integration. UPLC/MS analyses were run on a Waters ACQUITY UPLC/MS system consisting of a SQD (single quadrupole detector) mass spectrometer equipped with an electrospray ionization interface and a photodiode array detector. The PDA range was 210-400 nm. Analyses were performed on an ACQUITY UPLC BEH C18 column (100×2.1 mmID, particle size 1.7 μm) with a VanGuard BEH C18 pre-column (5×2.1 mmID, particle size 1.7 μm). Mobile phase was 10 mM NH4OAc in H₂O at pH 5 adjusted with CH₃COOH (A) and 10 mM NH4OAc in CH₃CN—H₂O (95:5) at pH 5.0. Three types of gradients were applied depending on the analysis, gradient 1 (5% to 100% mobile phase B in 3 min), gradient 2 (5% to 50% mobile phase B in 3 min) or gradient 3 (50% to 100% mobile phase B in 3 min). Electrospray ionization in positive and negative mode was applied. Electrospray ionization in positive and negative mode was applied. ESI was applied in positive and negative mode. All tested compounds showed ≥90% purity by NMR and UPLC/MS analysis.

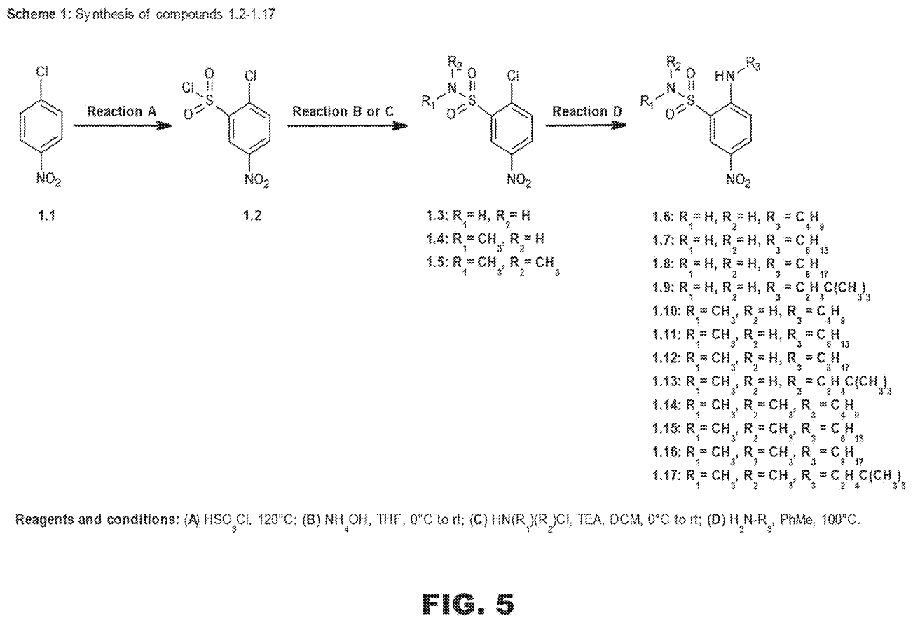

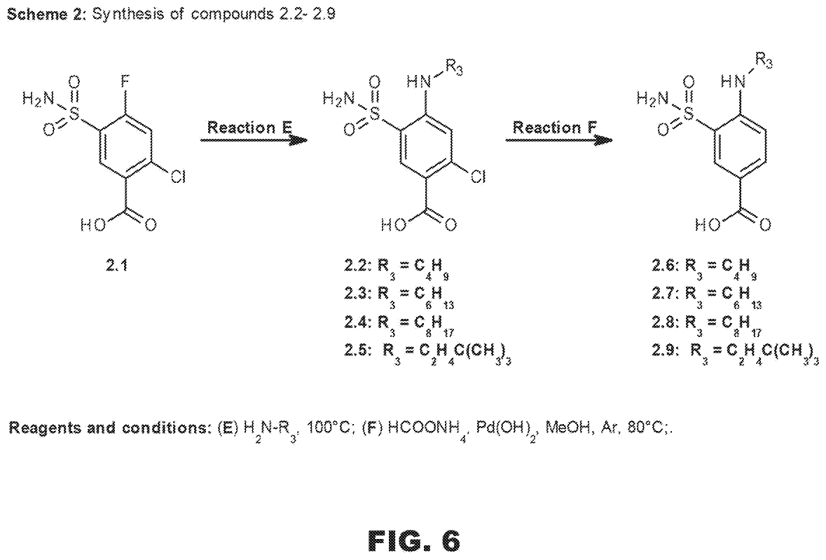

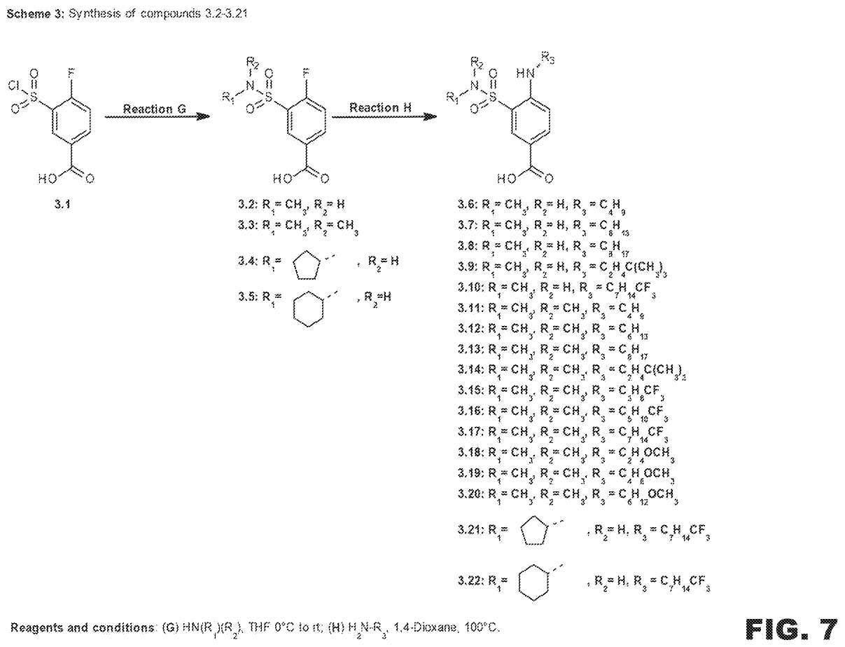

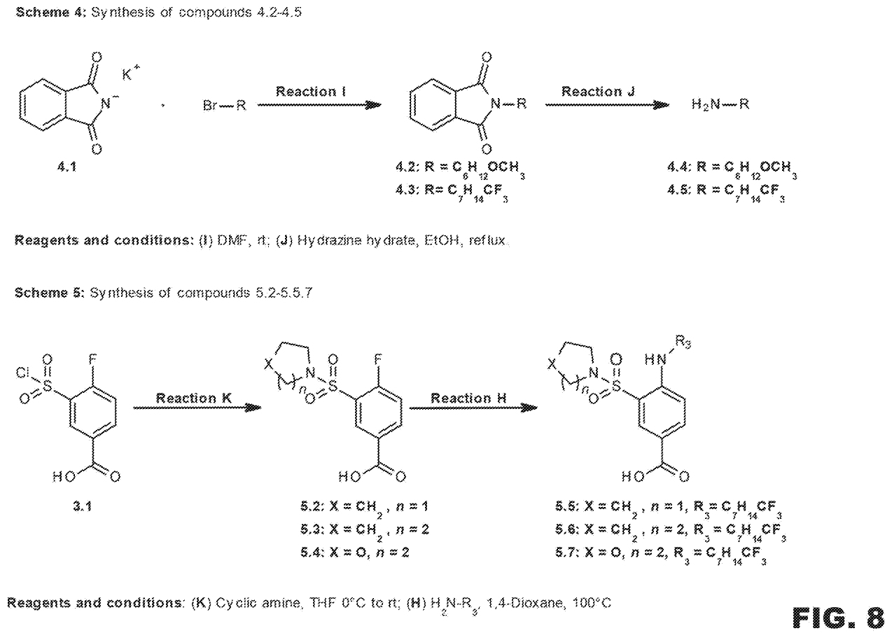

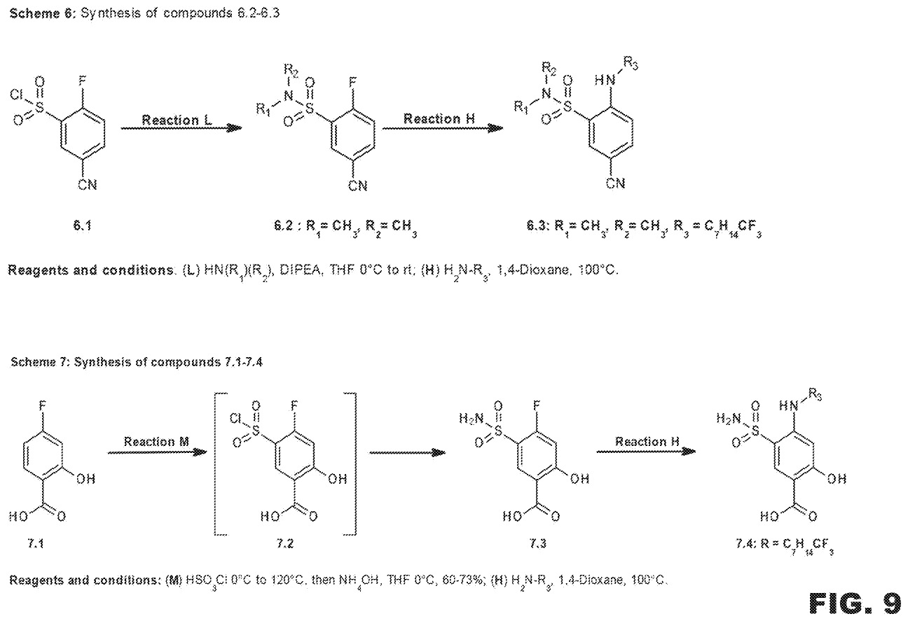

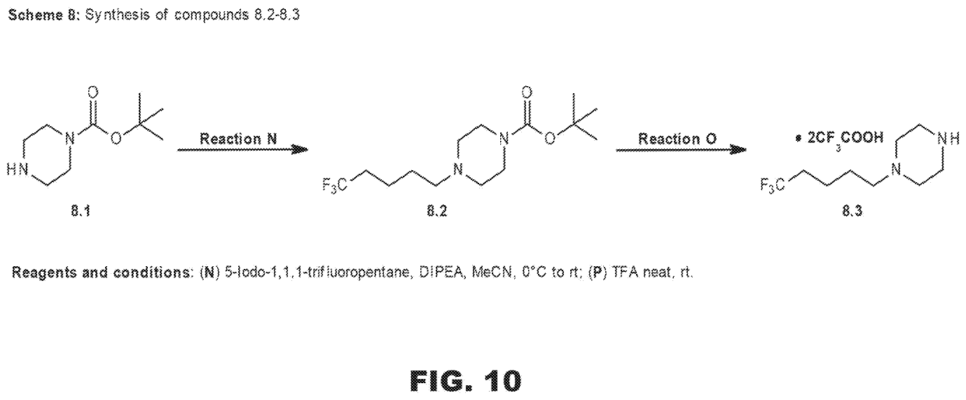

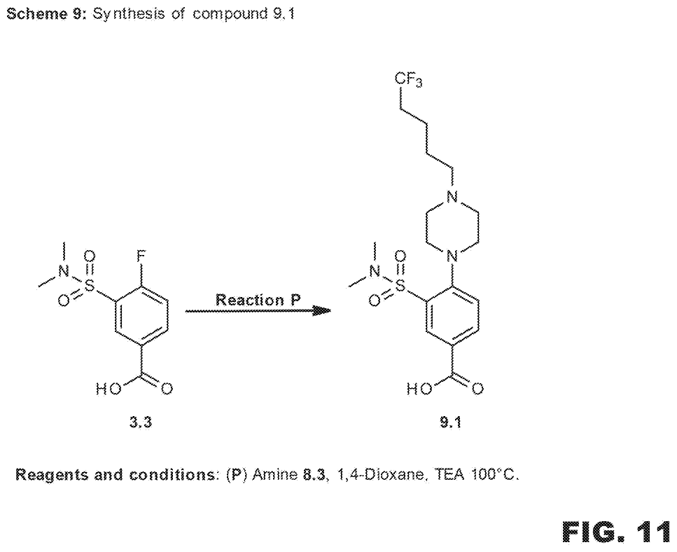

Schemes and synthetic procedures for preparing some of the compounds of the invention are depicted in FIGS. 5A to 5D.

### Synthesis

### 2-chloro-5-nitro-benzenesulfonyl chloride (Compound 1.2, Scheme 1)

1-Chloro-4-nitrobenzene 1.1 (500 mg, 3.14 mmol) was stirred in chlorosulfonic acid (1.05 ml, 15.71 mmol) at 120° C. for 16 h. At reaction completion the mixture was slowly poured onto ice-cold water (30 ml), and extracted twice with DCM (2×30 ml). The combined organic layers were dried over Na₂SO₄ and concentrated to dryness at low pressure to afford 374.1 (yield 46%) mg of titled compound. Characterization: Rt=2.14 min; MS (ESI) m/z: 253.7 [M−H]−, [M−H]− calculated: 254.9.1H NMR (400 MHz, DMSO-d6) δ 8.61 (d, J=2.9 Hz, 1H), 8.16 (dd, J=8.7, 2.9 Hz, 1H), 7.70 (d, J=8.6 Hz, 1H).

### 2-chloro-5-nitro-benzenesulfonamide (Compound 1.3, Scheme 1)

To an ice-cold solution of 5 ml tetrahydrofuran and 4 ml of 20% aqueous NH₄OH was added compound 1.2 (374.1, 1.47 mmol) solved in THF and the reaction mixture was stirred at room temperature for 1 hour. The reaction crude was then evaporated to dryness at low pressure, and the residue suspended in water (20 ml) and extracted twice with EtOAc (2×20 ml). The combined organic layers were dried over Na₂SO₄ and concentrated to dryness at low pressure. Purification by silica gel flash chromatography (cyclohexane/EtOAc from 90:10 to 70:30) afforded the pure titled compound (166.2 g, yield 48%). Characterization: Rt=1.42 min; MS (ESI) m/z: 235.3 [M−H]−, [M−H]− calculated: 236. 1H NMR (400 MHz, DMSO-d6) δ 8.68 (d, J=2.7 Hz, 1H), 8.42 (dd, J=8.7, 2.8 Hz, 1H), 7.98 (s, 2H), 7.96 (m, J=8.7 Hz, 1H).

### General Procedure C for the Synthesis of Sulfonamides 1.4-1.5 (Reaction C, Scheme 1)

To an ice-cold solution of proper amine hydrochloride (1.0 mmol) and triethylamine (2 mmol) in DCM (1.0 ml) was added compound 1.2 (1 mmol) solved in DCM (1.5 ml) and the reaction mixture was stirred at room temperature for 1 hour. The reaction crude was diluted with DCM (20 ml) and washed with an NH₄Cl saturated solution (20 ml) and the aqueous layer was extracted twice with DCM (2×20 ml). The combined organic layers were dried over Na₂SO₄ and concentrated to dryness at low pressure. Purification by silica gel flash chromatography finally afforded the pure titled compounds.

### 2-chloro-N-methyl-5-nitro-benzenesulfonamide (Compound 1.4, Scheme 1)

Titled compound was synthesized following the general procedure C previously described using intermediate 1.2 (347 mg, 1.46 mmol) and methylamine hydrochloride (100.7 mg, 1.46 mmol). Purification by silica gel flash chromatography (cyclohexane/TBME 95:05) afforded the pure titled compound (204.9 mg, yield 56%). Characterization: Rt=1.62 min; MS (ESI) m/z: 249.3 [M−H]−. [M−H]− calculated: 250. 1H NMR (400 MHz, DMSO-d6) δ 8.61 (d, J=2.7 Hz, 1H), 8.45 (dd, J=8.7, 2.8 Hz, 1H), 8.11 (q, J=4.4 Hz, 1H), 2.53 (d, J=4.7 Hz, 3H).

### 2-chloro-N,N-dimethyl-5-nitro-benzenesulfonamide (Compound 1.5, Scheme 1)

Titled compound was synthesized following the general procedure C previously described using intermediate 1.2 (190.3 mg, 0.8 mmol) and dimethylamine hydrochloride (163.7 mg, 1.60 mmol). Purification by silica gel flash chromatography (cyclohexane/EtOAc 80:20) afforded the pure titled compound (156.32 mg, yield 74%). Characterization: Rt=1.98 min; MS (ESI) m/z: 265.3 [M−H]+. [M−H]− calculated: 264. 1H NMR (400 MHz, DMSO-d6) δ 8.59 (d, J=2.7 Hz, 1H), 8.46 (dd, J=8.7, 2.8 Hz, 1H), 8.01 (d, J=8.7 Hz, 1H), 2.87 (s, 6H).

### General Procedure D for the Synthesis of Compounds 1.6-1.17 (Reaction D, Scheme 1)

A suspension of intermediate 1.3, 1.4, or 1.5 (1 mmol) and the appropriate amine (5 mmol) in dry toluene (0.7 ml) was stirred under Argon atmosphere at 100° C. for 1 hour. After reaction completion the mixture was the evaporated to dryness at low pressure and the residue was treated with water (10 ml) and extracted with EtOAc (10 ml). The organic layer was dried over Na₂SO₄ and concentrated to dryness at low pressure. Purification by silica gel flash chromatography finally afforded the pure titled compounds.

### 2-(butylamino)-5-nitro-benzenesulfonamide (Compound 1.6, Scheme 1)

Titled compound was synthesized following the general procedure D previously described using intermediate 1.3 (50 mg, 0.21 mmol) and Butylamine (0.1 ml, 1.05 mmol). The compound was obtained pure without silica gel purification (55.96 mg, yield 97%). Characterization: Rt=2.03 min; MS (ESI) m/z: 274.4 [M−H]+. [M−H]− calculated: 273.1; 1H NMR (400 MHz, DMSO-d6) δ 8.48 (d, J=2.7 Hz, 1H), 8.19 (dd, J=9.4, 2.7 Hz, 1H), 6.95 (d, J=9.4 Hz, 1H), 3.35 (m, 2H), 1.65-1.55 (m, 2H), 1.44-1.32 (m, 2H), 0.92 (t, J=7.3 Hz, 3H).

### 2-(hexylamino)-5-nitro-benzenesulfonamide (Compound 1.7, Scheme 1)

Titled compound was synthesized following the general procedure D previously described using intermediate 1.3 (50 mg, 0.21 mmol) and Hexylamine (0.14 ml, 1.05 mmol). Purification by silica gel flash chromatography (cyclohexane/EtOAc from 90:10 to 70:30) afforded the pure titled compound (59.81 mg, yield 94%). Characterization: Rt=2.34 min; MS (ESI) m/z: 302.5 [M−H]+. [M−H]− calculated: 301.1; 1H NMR (400 MHz, DMSO-d6) δ 8.49 (d, J=2.7 Hz, 1H), 8.19 (ddd, J=9.4, 2.8, 0.5 Hz, 1H), 7.72 (s, 2H), 6.95 (d, J=9.4 Hz, 1H), 6.85 (t, J=5.6 Hz, 1H), 3.37-3.28 (m, 2H), 1.66-1.56 (m, 2H), 1.41-1.25 (m, 6H), 0.90-0.83 (m, 3H).

### 5-nitro-2-(octylamino)benzenesulfonamide (Compound 1.8, Scheme 1)

Titled compound was synthesized following the general procedure D previously described using intermediate 1.3 (50 mg, 0.21 mmol) and octylamine (0.175 ml, 1.05 mmol). Purification by silica gel flash chromatography (cyclohexane/EtOAc 80:20) afforded the pure titled compound (64.27 mg, yield 93%). Characterization: Rt=2.61 min; MS (ESI) m/z: 330.5 [M−H]+. [M−H]− calculated: 329.1; 1H NMR (400 MHz, DMSO-d6) δ 8.49 (d, J=2.8 Hz, 1H), 8.20 (dd, J=9.4, 2.8 Hz, 1H), 7.73 (s, 2H), 6.95 (d, J=9.4 Hz, 1H), 6.86 (s, 1H), 3.34-3.29 (m, 2H), 1.62 (p, J=7.2 Hz, 2H), 1.41-1.20 (m, 10H), 0.90-0.81 (m, 3H).

### 2-(3,3-dimethylbutylamino)-5-nitro-benzenesulfonamide (Compound 1.9, Scheme 1)

Title compound was synthesized following the general procedure D previously described using intermediate 1.3 (50 mg, 0.21 mmol) and 3,3-dimethylbutan-1-amine (0.148 ml, 1.05 mmol). Purification by silica gel flash chromatography (cyclohexane/EtOAc from 95:05 to 75:25) afforded the pure title compound (55.6 mg, yield 88%). Characterization: Rt=2.29 min; MS (ESI) m/z: 265.3 [M−H]+. [M−H]− calculated: 264; 1H NMR (400 MHz, DMSO-d6) δ 8.48 (d, J=2.7 Hz, 1H), 8.21 (dd, J=9.4, 2.8 Hz, 1H), 7.70 (s, 2H), 6.93 (d, J=9.4 Hz, 1H), 6.78 (t, J=4.7 Hz, 1H), 3.38-3.30 (m, 2H), 1.59-1.51 (m, 2H), 0.96 (s, 9H).

### 2-(butylamino)-N-methyl-5-nitro-benzenesulfonamide (Compound 1.10, Scheme 1)

Titled compound was synthesized following the general procedure D previously described using intermediate 1.4 (40 mg, 0.16 mmol) and Butylamine (80 μl, 0.79 mmol). Purification by silica gel flash chromatography (cyclohexane/EtOAc 80:20) afforded the pure titled compound (38.65 mg, yield 84%). Characterization: Rt=2.27 min; MS (ESI) m/z: 288.4 [M−H]+. [M−H]− calculated: 287.1; 1H NMR (400 MHz, DMSO-d6) δ 8.40 (d, J=2.8 Hz, 1H), 8.21 (dd, J=9.4, 2.7 Hz, 1H), 7.89 (s, 1H), 6.98 (d, J=9.4 Hz, 1H), 6.88 (t, J=5.6 Hz, 1H), 3.38-3.33 (m, 2H), 2.44 (s, 3H), 1.66-1.54 (m, 2H), 1.43-1.32 (m, 2H), 0.92 (t, J=7.4 Hz, 3H).

### 2-(hexylamino)-N-methyl-5-nitro-benzenesulfonamide (Compound 1.11, Scheme 1)

Titled compound was synthesized following the general procedure D previously described using intermediate 1.4 (40 mg, 0.16 mmol) and hexylamine (0.1 ml, 0.79 mmol). Purification by silica gel flash chromatography (cyclohexane/EtOAc 80:20) afforded the pure titled compound (40.38 mg, yield 80%). Characterization: Rt=2.56 min; MS (ESI) m/z: 316.4 [M−H]+. [M−H]− calculated: 315.1; 1H NMR (400 MHz, DMSO-d6) δ 8.40 (d, J=2.8 Hz, 1H), 8.21 (dd, J=9.4, 2.8 Hz, 1H), 7.88 (s, 1H), 6.97 (d, J=9.5 Hz, 1H), 6.92 (t, J=5.6 Hz, 1H), 3.38-3.27 (m, 2H), 2.44 (s, 3H), 1.66-1.54 (m, 2H), 1.40-1.24 (m, 6H), 0.90-0.82 (m, 3H).

### N-methyl-5-nitro-2-(octylamino)benzenesulfonamide (Compound 1.12, Scheme 1)

Titled compound was synthesized following the general procedure D previously described using intermediate 1.4 (40 mg, 0.16 mmol) and octylamine (0.13 ml, 0.79 mmol)./Purification by silica gel flash chromatography (cyclohexane/EtOAc 80:20) afforded the pure titled compound (39.56 mg, yield 72%). Characterization: Rt=1.99 min; MS (ESI) m/z: 344.4 [M−H]+. [M−H]− calculated: 343.1; 1H NMR (400 MHz, DMSO-d6) δ 8.41 (d, J=2.8 Hz, 1H), 8.22 (dd, J=9.4, 2.8 Hz, 1H), 7.89 (s, 1H), 6.98 (d, J=9.4 Hz, 1H), 6.89 (t, J=5.5 Hz, 1H), 3.36-3.30 (m, 2H), 2.45 (s, 3H), 1.65-1.56 (m, 2H), 1.40-1.20 (m, 10H), 0.89-0.82 (m, 3H).

### 2-(3,3-dimethylbutylamino)-N-methyl-5-nitro-benzenesulfonamide (Compound 1.13, Scheme 1)

Titled compound was synthesized following the general procedure D previously described using intermediate 1.4 (40 mg, 0.16 mmol) and 3,3-dimethylbutan-1-amine (0.11 ml, 0.79 mmol). Purification by silica gel flash chromatography (cyclohexane/EtOAc 80:20) afforded the pure titled compound (42.26 mg, yield 84%). Characterization: Rt=2.15 min; MS (ESI) m/z: 316.4 [M−H]+. [M−H]− calculated: 315.1; 1H NMR (400 MHz, DMSO-d6) δ 8.40 (d, J=2.7 Hz, 1H), 8.23 (dd, J=9.3, 2.8 Hz, 1H), 6.96 (d, J=9.4 Hz, 1H), 6.81 (t, J=5.4 Hz, 1H), 3.36-3.30 (m, 2H), 2.43 (s, 3H), 1.57-1.51 (m, 2H), 0.96 (s, 9H).

### 2-(butylamino)-N,N-dimethyl-5-nitro-benzenesulfonamide (Compound 1.14, Scheme 1)

Title compound was synthesized following the general procedure D previously described using intermediate 1.5 (50 mg, 0.19 mmol) and butylamine (93 μl, 0.94 mmol). Purification by silica gel flash chromatography (cyclohexane/EtOAc 75:25) afforded the pure title compound (41.45 mg, yield 72%). Characterization: Rt=2.47 min; MS (ESI) m/z: 302.4 [M−H]+. [M−H]− calculated: 301.1; 1H NMR (400 MHz, DMSO-d6) δ 8.29 (d, J=2.8 Hz, 1H), 8.25 (ddd, J=9.4, 2.7, 0.6 Hz, 1H), 7.21 (t, J=5.6 Hz, 1H), 7.03 (d, J=9.5 Hz, 1H), 3.38-3.32 (m, 2H), 2.72 (s, 6H), 1.63-1.53 (m, 2H), 1.42-1.32 (m, 2H), 0.93 (t, J=7.3 Hz, 3H).

### 2-(hexylamino)-N,N-dimethyl-5-nitro-benzenesulfonamide (Compound 1.15, Scheme 1)

Titled compound was synthesized following the general procedure D previously described using intermediate 1.5 (65 mg, 0.24 mmol) and hexylamine (0.16 ml, 1.21 mmol). Purification by silica gel flash chromatography (cyclohexane/EtOAc 80:20) afforded the pure titled compound (68.42 mg, yield 87%). Characterization: Rt=1.80 min; MS (ESI) m/z: 328.5 [M−H]−. [M−H]− calculated: 329.1; 1H NMR (400 MHz, DMSO-d6) δ 8.28 (d, J=2.7 Hz, 1H), 8.24 (ddd, J=9.4, 2.8, 0.6 Hz, 1H), 7.21 (t, J=5.6 Hz, 1H), 7.01 (d, J=9.4 Hz, 1H), 3.36-3.30 (m, 2H), 2.71 (s, 6H), 1.62-1.53 (m, 2H), 1.38-1.24 (m, 6H), 0.90-0.82 (m, 3H).

### N,N-dimethyl-5-nitro-2-(octylamino)benzenesulfonamide (Compound 1.16, Scheme 1)

Titled compound was synthesized following the general procedure D previously described using intermediate 1.5 (50 mg, 0.19 mmol) and octylamine (0.15 ml, 0.94 mmol). Purification by silica gel flash chromatography (cyclohexane/EtOAc 85:15) afforded the pure titled compound (57.52 mg, yield 85%). Characterization: Rt=2.30 min; MS (ESI) m/z: 358.4 [M−H]+. [M−H]− calculated: 357.2; 1H NMR (400 MHz, DMSO-d6) δ 8.28 (d, J=2.8 Hz, 1H), 8.23 (ddd, J=9.4, 2.8, 0.6 Hz, 1H), 7.20 (t, J=5.6 Hz, 1H), 7.01 (d, J=9.5 Hz, 1H), 3.38-3.31 (m, 2H), 2.71 (s, 6H), 1.62-1.53 (m, 2H), 1.37-1.20 (m, 10H), 0.87-0.82 (m, 3H).

### 2-(3,3-dimethylbutylamino)-N,N-dimethyl-5-nitro-benzenesulfonamide (Compound 1.17, Scheme 1)

Titled compound was synthesized following the general procedure D previously described using intermediate 1.5 (50 mg, 0.19 mmol) and 3,3-dimethylbutan-1-amine (0.13 ml, 0.94 mmol). Purification by silica gel flash chromatography (cyclohexane/EtOAc 85:15) afforded the pure titled compound (51.11 mg, yield 82%). Characterization: Rt=2.70 min; MS (ESI) m/z: 330.4 [M−H]+. [M−H]− calculated: 329.1; 1H NMR (400 MHz, DMSO-d6) δ 8.28 (d, J=2.7 Hz, 1H), 8.25 (ddd, J=9.3, 2.8, 0.6 Hz, 1H), 7.16 (t, J=5.6 Hz, 1H), 6.98 (d, J=9.3 Hz, 1H), 3.38-3.32 (m, 2H), 2.71 (s, 6H), 1.52-1.47 (m, 2H), 0.95 (s, 9H).

### General Procedure E for the Synthesis of Compounds 2.2-2.5 (Scheme 2)

A suspension of commercial 2-chloro-4-fluoro-5-sulfamoyl-benzoic acid 2.1 (1 mmol) and the appropriate amine (5 mmol) in dry toluene (0.7 ml) was stirred under Argon atmosphere at 100° C. for 1 hour. After reaction completion the mixture was evaporated to dryness at low pressure and the residue was treated with a saturated NH₄Cl aqueous solution (15 ml) and extracted with EtOAc (15 ml). The combined organic layers were dried over Na₂SO₄ and concentrated to dryness at low pressure. Trituration in cyclohexane afforded finally the pure title compounds.

### 4-(butylamino)-2-chloro-5-sulfamoyl-benzoic acid (Compound 2.2, Scheme 2)

Titled compound was synthesized following the general procedure E previously described using intermediate 2.1 (70 mg, 0.26 mmol) and butylamine (0.13 ml, 1.32 mmol). Trituration with cyclohexane (1 ml) afforded the pure titled compound (40.84 mg, yield 51%). Characterization: Rt=1.52 min; MS (ESI) m/z: 305.3 [M−H]−. [M−H]− calculated: 306.04; 1H NMR (400 MHz, DMSO-d6) δ 12.80 (bs, 1H), 8.26 (s, 1H), 7.57 (s, 2H), 6.84 (s, 1H), 6.39 (t, J=5.3 Hz, 1H), 3.31-3.21 (m, 2H), 1.64-1.53 (m, 2H), 1.44-1.33 (m, 2H), 0.93 (t, J=7.3 Hz, 3H).

### 2-chloro-4-(hexylamino)-5-sulfamoyl-benzoic acid (Compound 2.3, Scheme 2)

Titled compound was synthesized following the general procedure E previously described using intermediate 2.1 (50 mg, 0.19 mmol) and hexylamine (0.12 ml, 0.95 mmol). Trituration with cyclohexane (1 ml) afforded the pure titled compound 52.82 mg, yield 83%). Characterization: Rt=1.78 min; MS (ESI) m/z: 333.4 [M−H]−. [M−H]− calculated: 334.1; 1H NMR (400 MHz, DMSO-d6) δ 12.77 (bs, 1H), 8.25 (s, 1H), 7.55 (s, 2H), 6.83 (s, 1H), 6.39 (t, J=5.4 Hz, 1H), 3.27-3.20 (m, 2H), 1.59 (p, J=7.1 Hz, 2H), 1.41-1.24 (m, 6H), 0.90-0.84 (m, 3H).

### 2-chloro-4-(octylamino)-5-sulfamoyl-benzoic acid (Compound 2.4, Scheme 2)

Titled compound was synthesized following the general procedure E previously described using intermediate 2.1 (50 mg, 0.19 mmol) and octylamine (0.16 ml, 0.95 mmol). Trituration with cyclohexane (1 ml) afforded the pure titled compound 48.89 mg, yield 71%). Characterization: Rt=2.01 min; MS (ESI) m/z: 361.4 [M−H]−. [M−H]− calculated: 362.1; 1H NMR (400 MHz, DMSO-d6) δ 12.78 (bs, 1H), 8.26 (s, 1H), 7.56 (s, 2H), 6.84 (s, 1H), 6.40 (t, J=5.3 Hz, 1H), 3.28-3.21 (m, 2H), 1.65-1.55 (m, 2H), 1.41-1.20 (m, 10H), 0.90-0.83 (m, 3H).

### 2-chloro-4-(3,3-dimethylbutylamino)-5-sulfamoyl-benzoic acid (Compound 2.5, Scheme 2)

Titled compound was synthesized following the general procedure E previously described using intermediate 2.1 (50 mg, 0.19 mmol) and 3,3-dimethylbutan-1-amine (0.13 ml, 0.95 mmol). Trituration with cyclohexane (1 ml) afforded the pure titled compound 52.82 mg, yield 83%). Characterization: Rt=1.66 min; MS (ESI) m/z: 333.4 [M−H]−. [M−H]− calculated: 334.1; 1H NMR (400 MHz, DMSO-d6) δ 8.25 (s, 1H), 7.54 (s, 2H), 6.83 (s, 1H), 6.29 (t, J=5.1 Hz, 1H), 3.27-3.20 (m, 2H), 1.56-1.50 (m, 2H), 0.96 (s, 9H).

### General Procedure F for the Synthesis of Compounds 2.6-2.9 (Reaction F, Scheme 2)

Under Ar atmosphere, to a suspension of the proper 4-amino-2-chloro-5-sulfamoyl-benzoic acid intermediates 2.2-2.5 (1 mmol) and Palladium hydroxide on carbon (20 wt. %) in dry methanol (20 ml), was added Ammonium formate (4 mmol) and the reaction mixture was stirred at reflux temperature for 1 hour. After reaction completion the crude was filtered through a celite coarse patch and the filtrate concentrated to dryness at low pressure. The dry residue was diluted in EtOAc (10 ml) and washed with a saturated NH₄Cl solution (10 ml). The organic layer was dried over Na₂SO₄ and concentrated to dryness at low pressure. Trituration in cyclohexane afforded finally the pure title compounds.

### 4-(butylamino)-3-sulfamoyl-benzoic acid (Compound 2.6, Scheme 2)

Titled compound was synthesized following the general procedure F previously described using intermediate 2.2 (30 mg, 0.1 mmol). Trituration with cyclohexane (1 ml) afforded the pure titled compound (11.71 mg, yield 43%). Characterization: Rt=1.53 min; MS (ESI) m/z: 273.4 [M−H]+. [M−H]− calculated: 272.1; 1H NMR (400 MHz, DMSO-d6) δ 8.23 (d, J=2.1 Hz, 1H), 7.87 (dd, J=8.8, 2.2 Hz, 1H), 7.46 (s, 2H), 6.83 (d, J=8.9 Hz, 1H), 6.37 (t, J=5.4 Hz, 1H), 3.28-3.21 (m, 2H), 1.64-1.55 (m, 2H), 1.44-1.34 (m, 2H), 0.92 (t, J=7.3 Hz, 3H).

### 4-(hexylamino)-3-sulfamoyl-benzoic acid (Compound 2.7, Scheme 2)

Titled compound was synthesized following the general procedure F previously described using intermediate 2.3 (30.7 mg, 0.09 mmol). Trituration with cyclohexane (1 ml) afforded the pure titled compound (11.71 mg, yield %). Characterization: Rt=1.81 min; MS (ESI) m/z: 301.4 [M−H]+. [M−H]− calculated: 300.1; 1H NMR (400 MHz, DMSO-d6) δ 12.45 (bs, 1H), 8.23 (d, J=2.1 Hz, 1H), 7.87 (dd, J=8.8, 2.2 Hz, 1H), 7.46 (s, 2H), 6.82 (d, J=8.9 Hz, 1H), 6.38 (t, J=5.4 Hz, 1H), 3.27-3.20 (m, 2H), 1.60 (h, J=6.6 Hz, 2H), 1.42-1.25 (m, 8H), 0.92-0.80 (m, 3H).

### 4-(octylamino)-3-sulfamoyl-benzoic acid (Compound 2.8, Scheme 2)

Titled compound was synthesized following the general procedure F previously described using intermediate 2.4 (35.7 mg, 0.1 mmol). Trituration with cyclohexane (1 ml) afforded the pure titled compound (9.68 mg, yield 36%). Characterization: Rt=2.16 min; MS (ESI) m/z: 329.4 [M−H]+. [M−H]− calculated: 328.1; 1H NMR (400 MHz, DMSO-d6) δ 12.43 (bs, 1H), 8.23 (d, J=2.1 Hz, 1H), 7.86 (dd, J=8.7, 2.1 Hz, 1H), 7.46 (s, 2H), 6.82 (d, J=8.9 Hz, 1H), 6.38 (t, J=5.3 Hz, 1H), 3.27-3.19 (m, 2H), 1.65-1.56 (m, 2H), 1.42-1.15 (m, 12H), 0.92-0.80 (m, 3H).

### 4-(3,3-dimethylbutylamino)-3-sulfamoyl-benzoic acid (Compound 2.9, Scheme 2)

Titled compound was synthesized following the general procedure F previously described using intermediate 2.5 (29.6 mg, 0.09 mmol). Trituration with cyclohexane (1 ml) afforded the pure titled compound (15.13 mg, yield 56%). Characterization: Rt=1.80 min; MS (ESI) m/z: 301.4 [M−H]+. [M−H]− calculated: 300.1; 1H NMR (400 MHz, DMSO-d6) δ 12.48 (bs, 1H), 8.24 (d, J=2.1 Hz, 1H), 7.89 (dd, J=8.8, 2.1 Hz, 1H), 7.46 (s, 2H), 6.83 (d, J=8.9 Hz, 1H), 3.28-3.21 (m, 2H), 1.59-1.52 (m, 2H), 0.97 (s, 9H).

### General Procedure G for the Synthesis of Compounds 3.2-3.3 (Reaction G, Scheme 3)

4-Fluoro-3-chlorosulfonyl-benzoic acid 3.1 (1 mmol) solved in 1.5 mL of THF was added dropwise to 3 mL of an ice cold 2 M solution of the proper amine in THF and stirred for 1 h at RT (Room Temperature). At reaction completion the reaction mixture was evaporated to dryness and the residue treated with water and HCl. The precipitated product was filtered and rinsed with water to afford the pure titled compounds.

### 4-fluoro-3-(methylsulfamoyl)benzoic acid (Compound 3.2, Scheme 3)

Titled compound was synthesized following the general procedure G previously described using intermediate 3.1 (500 mg, 2.07 mmol) and a 2M methylamine solution in THF (2.07 ml, 4.15 mmol). The described workup afforded pure titled compound (313.8 mg, yield 64%). Characterization: Rt=1.26 min; MS (ESI) m/z: 232.3 [M−H]−. [M−H]− calculated: 233.02 1H NMR (400 MHz, DMSO-d6) δ 8.30 (dd, J=7.0, 2.2 Hz, 1H), 8.25-8.19 (m, 1H), 7.89 (q, J=4.8 Hz, 1H), 7.62-7.54 (m, 1H), 2.52 (d, J=4.8 Hz, 3H).

### 3-(dimethylsulfamoyl)-4-fluoro-benzoic acid (Compound 3.3, Scheme 3)

Titled compound was synthesized following the general procedure G previously described using intermediate 3.1 (1 g, 4.15 mmol) and a 2M dimethylamine solution in THF (4.15 ml, 8.30 mmol). The described workup afforded pure titled compound (749 mg, yield 73%). Characterization: Rt=1.11 min; MS (ESI) m/z: 246.3 [M−H]−. [M−H]− calculated: 247.03. 1H NMR (400 MHz, DMSO-d6) δ 8.29-8.24 (m, 2H), 7.67-7.58 (m, 1H), 2.75 (d, J=1.9 Hz, 6H).

### 3-(cyclopentylsulfamoyl)-4-fluoro-benzoic acid (Compound 3.4, Scheme 3)

Titled compound was synthesized following the general procedure G previously described using intermediate 3.1 (250 mg, 1.04 mmol) and cyclopentyl amine (0.21 ml, 2.07 mmol) in THF (8.5 ml). The described workup afforded pure titled compound (261.4 mg, yield 88%). Characterization: Rt=1.25 min; MS (ESI) m/z: 286.4 [M−H]−. [M−H]− calculated: 287.06. 1H NMR (400 MHz, DMSO-d6) δ 8.33 (dd, J=7.1, 2.3 Hz, 1H), 8.21 (ddd, J=8.6, 4.7, 2.3 Hz, 1H), 8.12 (d, J=7.6 Hz, 1H), 7.56 (dd, J=10.0, 8.6 Hz, 1H), 3.58-3.48 (m, 1H), 1.68-1.48 (m, 4H), 1.45-1.28 (m, 4H).

### 3-(cyclohexylsulfamoyl)-4-fluoro-benzoic acid (Compound 3.5, Scheme 3)

Titled compound was synthesized following the general procedure G previously described using intermediate 3.1 (250 mg, 1.04 mmol) and cyclohexyl amine (0.24 ml, 2.07 mmol) in THF (8.5 ml). The described workup and trituration with a cyclohexane/ethyl acetate 9:1 mixture (2 ml) afforded pure titled compound (185.6 mg, yield 59%). Characterization: Rt=1.37 min; MS (ESI) m/z: 286.4 [M−H]−. [M−H]− calculated: 287.06. 1H NMR (400 MHz, DMSO-d6) δ 8.33 (dd, J=7.1, 2.3 Hz, 1H), 8.21 (ddd, J=8.6, 4.7, 2.3 Hz, 1H), 8.12 (d, J=7.6 Hz, 1H), 7.56 (dd, J=10.0, 8.6 Hz, 1H), 3.58-3.48 (m, 1H), 1.68-1.48 (m, 4H), 1.45-1.28 (m, 4H).

### General Procedure H for the Synthesis of Compounds 3.6-3.22, 5.5-5.7, 6.3, 7.4 (Reaction H, Scheme 3,5,6,7)

A suspension of the appropriate intermediate (1 mmol) and the appropriate amine (2 mmol) in dry 1,4-dioxane (3 ml) was stirred under Argon atmosphere at 100° C. for 4 hours. After reaction completion the mixture was evaporated to dryness at low pressure and the residue was treated with a saturated NH₄Cl aqueous solution (15 ml) and extracted twice with EtOAc (2×15 ml). The combined organic layers were dried over Na₂SO₄ and concentrated to dryness at low pressure. Trituration in cyclohexane afforded finally the pure title compounds.

### 4-(butylamino)-3-(methylsulfamoyl)benzoic acid (Compound 3.6, Scheme 3)

Titled compound was synthesized following the general procedure H previously described using intermediate 3.2 (50 mg, 0.21 mmol) and butylamine (42 μl, 0.42 mmol) in dry 1,4-Dioxane (0.7 ml). Trituration with cyclohexane (1 ml) afforded the pure titled compound (47.10 mg, yield 78%). Characterization: Rt=1.66 min; MS (ESI) m/z: 285.4 [M−H]−. [M−H]− calculated: 286.1. 1H NMR (400 MHz, DMSO-d6) δ 8.15 (d, J=2.1 Hz, 1H), 7.90 (dd, J=8.8, 2.1 Hz, 1H), 7.66 (s, 1H), 6.86 (d, J=8.9 Hz, 1H), 6.44 (t, J=5.4 Hz, 1H), 3.24 (q, J=6.6 Hz, 2H), 2.39 (s, 3H), 1.58 (p, J=7.2 Hz, 2H), 1.43-1.32 (m, 2H), 0.92 (t, J=7.3 Hz, 3H).

### 4-(hexylamino)-3-(methylsulfamoyl)benzoic acid (Compound 3.7, Scheme 3)

Titled compound was synthesized following the general procedure H previously described using intermediate 3.2 (50 mg, 0.21 mmol) and hexylamine (57 μl, 0.42 mmol) in dry 1,4-Dioxane (0.7 ml). Trituration with cyclohexane (1 ml) afforded the pure titled compound (51.69 mg, yield 78%). Characterization: Rt=2.00 min; MS (ESI) m/z: 313.4 [M−H]−. [M−H]− calculated: 314.1. 1H NMR (400 MHz, DMSO-d6) δ 12.53 (bs, 1H), 8.15 (d, J=2.1 Hz, 1H), 7.90 (dd, J=8.8, 2.1 Hz, 1H), 7.63 (q, J=5.0 Hz, 1H), 6.86 (d, J=8.9 Hz, 1H), 6.44 (t, J=5.3 Hz, 1H), 3.23 (q, J=6.6 Hz, 2H), 1.60 (p, J=7.1 Hz, 2H), 1.40-1.25 (m, 6H), 0.90-0.83 (m, 3H).

### 3-(methylsulfamoyl)-4-(octylamino)benzoic acid (Compound 3.8, Scheme 3)

Titled compound was synthesized following the general procedure H previously described using intermediate 3.2 (50 mg, 0.21 mmol) and octylamine (71 μl, 0.42 mmol) in dry 1,4-Dioxane (0.7 ml). Trituration with cyclohexane (1 ml) afforded the pure titled compound (69.51 mg, yield 97%). Characterization: Rt=2.28 min; MS (ESI) m/z: 341.4 [M−H]−. [M−H]− calculated: 342.2. 1H NMR (400 MHz, DMSO-d6) δ 8.15 (d, J=2.1 Hz, 1H), 7.89 (dd, J=8.8, 2.1 Hz, 1H), 6.86 (d, J=8.9 Hz, 1H), 6.44 (t, J=5.4 Hz, 1H), 3.23 (q, J=6.6 Hz, 2H), 2.38 (s, 3H), 1.59 (p, J=7.1 Hz, 2H), 1.40-1.20 (m, 9H), 0.89-0.82 (m, 3H).

### 4-(3,3-dimethylbutylamino)-3-(methylsulfamoyl)benzoic acid (Compound 3.9, Scheme 3)

Titled compound was synthesized following the general procedure H previously described using intermediate 3.2 (50 mg, 0.21 mmol) and 3,3-dimethylbutan-1-amine (60 μl, 0.42 mmol) in dry 1,4-Dioxane (0.7 ml). Trituration with cyclohexane (1 ml) afforded the pure titled compound (50.56 mg, yield 84%). Characterization: Rt=1.93 min; MS (ESI) m/z: 313.4 [M−H]−. [M−H]− calculated: 314.1. 1H NMR (400 MHz, DMSO-d6) δ 12.52 (s, 1H), 8.15 (d, J=2.1 Hz, 1H), 7.91 (dd, J=8.8, 2.1 Hz, 1H), 7.62 (q, J=5.0 Hz, 1H), 6.86 (d, J=8.9 Hz, 1H), 6.35 (t, J=5.2 Hz, 1H), 3.27-3.20 (m, 2H), 2.38 (d, J=5.0 Hz, 3H), 1.57-1.50 (m, 2H), 0.96 (s, 9H).

### 3-(methylsulfamoyl)-4-(8,8,8-trifluorooctylamino)benzoic acid (Compound 3.10, Scheme 3)

Titled compound was synthesized following the general procedure H previously described using intermediate 3.2 (100 mg, 0.42 mmol) and intermediate 4.5 (86.4 mg, 0.47 mmol) in dry 1,4-Dioxane (1.4 ml). Trituration with cyclohexane (2 ml) afforded the pure titled compound (111.5 mg, yield 67%). Characterization: Rt=2.11 min; MS (ESI) m/z: 395.2 [M−H]−. [M−H]− calculated: 396.1. 1H NMR (400 MHz, DMSO-d6) δ 8.15 (d, J=2.1 Hz, 1H), 7.90 (dd, J=8.8, 2.1 Hz, 1H), 7.63 (q, J=5.0 Hz, 1H), 6.86 (d, J=8.9 Hz, 1H), 6.44 (t, J=5.4 Hz, 1H), 3.24 (q, J=6.7 Hz, 2H), 2.39 (d, J=4.8 Hz, 3H), 2.28-2.15 (m, 2H), 1.64-1.55 (m, 2H), 1.51-1.42 (m, 2H), 1.39-1.30 (m, 6H).

### 4-(butylamino)-3-(dimethylsulfamoyl)benzoic acid (Compound 3.11, Scheme 3)

Titled compound was synthesized following the general procedure H previously described using intermediate 3.3 (50 mg, 0.20 mmol) and butylamine (40 μl, 0.40 mmol) in dry 1,4-Dioxane (0.7 ml). Trituration with cyclohexane (1 ml) afforded the pure titled compound (41.45 mg, yield 69%). Characterization: Rt=1.90 min; MS (ESI) m/z: 299.4 [M−H]−. [M−H]− calculated: 300.1. 1H NMR (400 MHz, DMSO-d6) δ 12.62 (s, 1H), 8.05 (d, J=2.1 Hz, 1H), 7.93 (dd, J=8.9, 2.1 Hz, 1H), 6.91 (d, J=9.0 Hz, 1H), 6.74 (t, J=5.4 Hz, 1H), 3.29-3.19 (m, 2H), 2.66 (s, 6H), 1.61-1.52 (m, 2H), 1.42-1.31 (m, 2H), 0.92 (t, J=7.3 Hz, 3H).

### 3-(dimethylsulfamoyl)-4-(hexylamino)benzoic acid (Compound 3.12, Scheme 3)

Titled compound was synthesized following the general procedure H previously described using intermediate 3.3 (50 mg, 0.20 mmol) and hexylamine (53 μl, 0.40 mmol) in dry 1,4-Dioxane (0.7 ml). Trituration with cyclohexane (1 ml) afforded the pure titled compound (53.20 mg, yield 81%). Characterization: Rt=2.17 min; MS (ESI) m/z: 327.4 [M−H]−. [M−H]− calculated: 328.1. 1H NMR (400 MHz, DMSO-d6) δ 12.63 (s, 1H), 8.04 (d, J=2.1 Hz, 1H), 7.93 (dd, J=8.8, 2.1 Hz, 1H), 6.90 (d, J=9.0 Hz, 1H), 6.74 (t, J=5.4 Hz, 1H), 3.28-3.18 (m, 2H), 2.65 (s, 6H), 1.57 (p, J=7.0 Hz, 2H), 1.39-1.24 (m, 6H), 0.89-0.84 (m, 3H).

### 3-(dimethylsulfamoyl)-4-(octylamino)benzoic acid (Compound 3.13, Scheme 3)

Titled compound was synthesized following the general procedure H previously described using intermediate 3.3 (50 mg, 0.20 mmol) and octylamine (67 μl, 0.40 mmol) in dry 1,4-Dioxane (0.7 ml). Trituration with cyclohexane (1 ml) afforded the pure titled compound (59.9 mg, yield 84%). Characterization: Rt=2.44 min; MS (ESI) m/z: 355.4 [M−H]−. [M−H]− calculated: 356.2. 1H NMR (400 MHz, DMSO-d6) δ 12.62 (s, 1H), 8.04 (d, J=2.1 Hz, 1H), 7.93 (dd, J=8.9, 2.1 Hz, 1H), 6.91 (d, J=9.0 Hz, 1H), 6.75 (t, J=5.4 Hz, 1H), 3.23 (q, J=6.6 Hz, 2H), 2.65 (s, 6H), 1.57 (p, J=6.9 Hz, 2H), 1.39-1.19 (m, 10H), 0.90-0.80 (m, 3H).

### 4-(3,3-dimethylbutylamino)-3-(dimethylsulfamoyl)benzoic acid (Compound 3.14, Scheme 3)

Titled compound was synthesized following the general procedure H previously described using intermediate 3.3 (50 mg, 0.20 mmol) and 3,3-dimethylbutan-1-amine (57 μl, 0.40 mmol) in dry 1,4-Dioxane (0.7 ml). Trituration with cyclohexane (1 ml) afforded the pure titled compound (42 mg, yield 63%). Characterization: Rt=2.13 min; MS (ESI) m/z: 327.4 [M−H]−. [M−H]− calculated: 328.1. 1H NMR (400 MHz, DMSO-d6) δ 12.63 (s, 1H), 8.05 (d, J=2.0 Hz, 1H), 7.95 (dd, J=8.9, 2.1 Hz, 1H), 6.90 (d, J=8.9 Hz, 1H), 6.69 (t, J=5.3 Hz, 1H), 3.29-3.22 (m, 2H), 2.66 (s, 6H), 1.54-1.46 (m, 2H), 0.96 (s, 9H).

### 3-(dimethylsulfamoyl)-4-(4,4,4 trifluorobutylamino) benzoic acid (Compound 3.15, Scheme 3)

Titled compound was synthesized following the general procedure H previously described using intermediate 3.3 (50 mg, 0.20 mmol) and 4,4,4-trifluorobutylamine (48 μl, 0.40 mmol) in dry 1,4-Dioxane (0.7 ml). Trituration with cyclohexane (1 ml) afforded the pure titled compound (40.13 mg, yield 57%). Characterization: Rt=1.78 min; MS (ESI) m/z: 353.4 [M−H]−. [M−H]− calculated: 354.1. 1H NMR (400 MHz, DMSO-d6) δ 12.64 (bs, 1H), 8.07 (d, J=2.1 Hz, 1H), 7.95 (dd, J=8.8, 2.1 Hz, 1H), 6.98 (d, J=9.0 Hz, 1H), 6.88 (t, J=5.9 Hz, 1H), 3.38 (q, J=6.8 Hz, 2H), 2.67 (s, 6H), 2.40-2.25 (m, 2H), 1.83-1.73 (m, 2H).

### 3-(dimethylsulfamoyl)-4-(6,6,6-trifluorohexylamino) benzoic acid (Compound 3.16, Scheme 3)

Titled compound was synthesized following the general procedure H previously described using intermediate 3.3 (50 mg, 0.20 mmol) and 6,6,6-trifluorohexylamine (60 μl, 0.40 mmol) in dry 1,4-Dioxane (0.7 ml). Trituration with cyclohexane (1 ml) afforded the pure titled compound (57.32 mg, yield 75%). Characterization: Rt=2.02 min; MS (ESI) m/z: 381.4 [M−H]−. [M−H]− calculated: 382.1. 1H NMR (400 MHz, DMSO-d6) δ 12.64 (bs, 1H), 8.05 (d, J=2.1 Hz, 1H), 7.94 (dd, J=8.8, 2.1 Hz, 1H), 6.93 (d, J=9.0 Hz, 1H), 6.77 (t, J=5.4 Hz, 1H), 3.26 (q, J=6.8 Hz, 2H), 2.66 (s, 6H), 2.32-2.18 (m, 2H), 1.62 (p, J=7.4 Hz, 3H), 1.58-1.48 (m, 2H), 1.47-1.37 (m, 2H).

### 3-(dimethylsulfamoyl)-4-(8,8,8-trifluorooctylamino) benzoic acid (Compound 3.17, Scheme 3)

Titled compound was synthesized following the general procedure H previously described using intermediate 3.3 (50 mg, 0.20 mmol) and intermediate 4.5 (89 mg, 0.40 mmol) in dry 1,4-Dioxane (0.7 ml). Trituration with cyclohexane (1 ml) afforded the pure titled compound (44.34 mg, yield 54%). Characterization: Rt=2.28 min; MS (ESI) m/z: 409.4 [M−H]−. [M−H]− calculated: 410.1. 1H NMR (400 MHz, DMSO-d6) δ 12.62 (s, 1H), 8.05 (d, J=2.1 Hz, 1H), 7.93 (dd, J=8.8, 2.1 Hz, 1H), 6.91 (d, J=9.0 Hz, 1H), 6.75 (t, J=5.4 Hz, 1H), 3.24 (q, J=6.6 Hz, 2H), 2.29-2.14 (m, 2H), 1.64-1.52 (m, 2H), 1.52-1.39 (m, 2H), 1.40-1.25 (m, 6H).

### 3-(dimethylsulfamoyl)-4-(2-methoxyethylamino)benzoic acid (Compound 3.18, Scheme 3)

Titled compound was synthesized following the general procedure H previously described using intermediate 3.3 (50 mg, 0.20 mmol) and 2-methoxyethylamine (36 μl, 0.40 mmol) in dry 1,4-Dioxane (0.7 ml). Trituration with cyclohexane (1 ml) afforded the pure titled compound (53.96 mg, yield 89%). Characterization: Rt=1.40 min; MS (ESI) m/z: 301.4 [M−H]−. [M−H]− calculated: 302.1. 1H NMR (400 MHz, DMSO-d6) δ 8.05 (d, J=2.1 Hz, 1H), 7.93 (dd, J=8.8, 2.1 Hz, 1H), 6.95 (d, J=9.0 Hz, 1H), 6.89 (t, J=5.3 Hz, 1H), 3.55 (t, J=5.2 Hz, 2H), 3.40 (q, J=5.3 Hz, 2H), 3.29 (s, 3H), 2.65 (s, 6H).

### 3-(dimethylsulfamoyl)-4-(4-methoxybutylamino)benzoic acid (Compound 3.19, Scheme 3)

Titled compound was synthesized following the general procedure H previously described using intermediate 3.3 (50 mg, 0.20 mmol) and 4-methoxybutan-1-amine (51 μl, 0.40 mmol) in dry 1,4-Dioxane (0.7 ml). Trituration with cyclohexane (1 ml) afforded the pure titled compound (56.08 mg, yield 85%). Characterization: Rt=1.59 min; MS (ESI) m/z: 329.4 [M−H]−. [M−H]− calculated: 330.1. 1H NMR (400 MHz, DMSO-d6) δ 12.63 (s, 1H), 8.05 (d, J=2.1 Hz, 1H), 7.93 (dd, J=8.8, 2.1 Hz, 1H), 6.91 (d, J=8.9 Hz, 1H), 6.77 (t, J=5.5 Hz, 1H), 3.38-3.32 (m, 2H), 3.26 (q, J=6.5 Hz, 2H), 3.22 (s, 3H), 2.65 (s, 6H), 1.65-1.51 (m, 4H).

### 3-(dimethylsulfamoyl)-4-(6-methoxyhexylamino)benzoic acid (Compound 3.20, Scheme 3)

Titled compound was synthesized following the general procedure H previously described using intermediate 3.3 (50 mg, 0.20 mmol) and intermediate 4.4 (53.1, 0.40 mmol) in dry 1,4-Dioxane (0.7 ml). Trituration with cyclohexane (1 ml) afforded the pure titled compound (23.17 mg, yield 32%). Characterization: Rt=1.84 min; MS (ESI) m/z: 357.5 [M−H]−. [M−H]− calculated: 358.2. 1H NMR (400 MHz, DMSO-d6) δ 8.04 (d, J=2.1 Hz, 1H), 7.93 (dd, J=8.8, 2.1 Hz, 1H), 6.90 (d, J=8.9 Hz, 1H), 6.73 (t, J=5.3 Hz, 1H), 3.31-3.26 (m, 2H), 3.26-3.21 (m, 2H), 3.20 (s, 3H), 1.62-1.53 (m, 2H), 1.52-1.43 (m, 2H), 1.39-1.27 (m, 4H).

### 3-(cyclopentylsulfamoyl)-4-(8,8,8-trifluorooctylamino) benzoic acid (Compound 3.21, Scheme 3)

Titled compound was synthesized following the general procedure H previously described using intermediate 3.4 (50 mg, 0.17 mmol) and intermediate 4.5 (35.1 mg, 0.19 mmol) in dry 1,4-Dioxane (0.6 ml). Trituration with diethyl ether (1 ml) afforded the pure titled compound (31.7 mg, yield 41%). Characterization: Rt=2.33 min; MS (ESI) m/z: 449.5 [M−H]−. [M−H]− calculated: 450.2. 1H NMR (400 MHz, Chloroform-d) δ 8.49 (d, J=2.1 Hz, 1H), 8.08 (dd, J=8.8, 2.1 Hz, 1H), 6.75 (d, J=8.9 Hz, 1H), 6.53 (s, 1H), 4.63-4.51 (m, 1H), 3.63-3.53 (m, 1H), 3.25 (t, J=7.1 Hz, 2H), 2.14-2.00 (m, 2H), 1.85-1.75 (m, 2H), 1.74-1.65 (m, 2H), 1.65-1.54 (m, 4H), 1.53-1.47 (m, 2H), 1.46-1.36 (m, 6H), 1.36-1.27 (m, 2H).

### 3-(cyclohexylsulfamoyl)-4-(8,8,8-trifluorooctylamino) benzoic acid (Compound 3.22, Scheme 3)

Titled compound was synthesized following the general procedure H previously described using intermediate 3.5 (50 mg, 0.16 mmol) and intermediate 4.5 (33.4 mg, 0.18 mmol) in dry 1,4-Dioxane (0.55 ml). Trituration with diethyl ether (1 ml) afforded the pure titled compound (25.3 mg, yield 34%). Characterization: Rt=2.40 min; MS (ESI) m/z: 463.5 [M−H]−. [M−H]− calculated: 464.2. 1H NMR (400 MHz, Chloroform-d) δ 8.49 (d, J=2.1 Hz, 1H), 8.07 (dd, J=8.8, 2.1 Hz, 1H), 6.74 (d, J=8.9 Hz, 1H), 6.50 (s, 1H), 4.49 (d, J=7.9 Hz, 1H), 3.25 (t, J=7.1 Hz, 2H), 3.18-3.07 (m, 1H), 2.14-2.00 (m, 2H), 1.79-1.66 (m, 4H), 1.66-1.49 (m, 6H), 1.48-1.34 (m, 6H), 1.30-1.19 (m, 3H), 1.18-1.07 (m, 2H).

### General Procedure I for the Synthesis of Intermediates 4.2-4.3 (Reaction I, Scheme 3)

A suspension of potassium phthalimide 4.1 (1 mmol) and the appropriate alkyl bromide (1.2 mmol) in dry N,N-dimethylformamide (3.5 ml) was stirred at room temperature for 16 hours. After reaction completion the mixture was diluted with water (35 ml) with EtOAc (35 ml). The organic layer was dried over Na₂SO₄ and concentrated to dryness at low pressure. Purification by silica gel flash chromatography finally afforded the pure titled compounds.

### 2-(6-methoxyhexyl)isoindoline-1,3-dione (Compound 4.2, Scheme 4)

Titled compound was synthesized following the general procedure I previously described, using potassium phthalimide 4.1 (300 mg, 1.60 mmol) and 1-Bromo-6-methoxyhexane (0.36 ml, 2.08 mmol) in dry N,N-dimethylformamide (5.5 ml). Purification by silica gel flash chromatography (cyclohexane/EtOAc 70:30) afforded the pure title compound (355.72 mg, yield 84%). Characterization: Rt=2.23 min; MS (ESI) m/z: 262.5 [M−H]+. [M−H]− calculated: 261.1. 1H NMR (400 MHz, Chloroform-d) δ 7.86-7.79 (m, 2H), 7.73-7.66 (m, 2H), 3.67 (t, J=7.4 Hz, 2H), 3.34 (t, J=6.5 Hz, 2H), 3.30 (s, 3H), 1.68 (p, J=6.1, 5.6 Hz, 2H), 1.56 (p, J=6.6 Hz, 2H), 1.43-1.31 (m, 4H).

### 2-(8,8,8-trifluorooctyl)isoindoline-1,3-dione (Compound 4.3, Scheme 4)

Titled compound was synthesized following the general procedure I previously described, using potassium phthalimide 4.1 (300 mg, 1.60 mmol) and intermediate 8-Bromo-1,1,1-trifluorooctane (0.4 ml, 2.08 mmol) in dry N,N-dimethylformamide (5.5 ml). Purification by silica gel flash chromatography (cyclohexane/EtOAc 85:15) afforded the pure title compound (392.63 mg, yield 75%). Characterization: Rt=1.76 min; MS (ESI) m/z: 314.4 [M−H]+. [M−H]− calculated: 313.1. 1H NMR (400 MHz, Chloroform-d) δ 7.86-7.81 (m, 2H), 7.73-7.67 (m, 2H), 3.70-3.65 (m, 2H), 2.11-1.97 (m, 2H), 1.68 (p, J=7.2 Hz, 2H), 1.58-1.47 (m, 2H), 1.39-1.30 (m, 6H).

### General Procedure J for the Synthesis of Compounds 4.4-4.5 (Reaction J, Scheme 4)

The corresponding intermediate 4.2 or 4.3 (1 mmol) was refluxed in absolute ethanol (1.2 mmol) with hydrazine hydrate (1.5 mmol) for 4 hours. At reaction completion, the mixture was cooled at room temperature and the resulting precipitated solid was filtered. The solid was washed with Ethanol and the filtrated concentrated to dryness at low pressure. Purification by basic alumina flash chromatography finally afforded the pure titled amines.

### 6-methoxyhexan-1-amine (Compound 4.4, Scheme 4)

Titled compound was synthesized following the general procedure J previously described, using intermediate 4.2 (356 mg, 1.35 mmol) and hydrazine hydrate (0.15 ml, 2.02 mmol) in absolute ethanol (5.5 ml). Purification by basic alumina flash chromatography (dichloromethane/methanol 90:10) afforded the pure title compound (127.55 mg, yield 7%). Characterization: Rt=1.00 min; MS (ESI) m/z: 132.4 [M−H]+. [M−H]− calculated: 131.1. 1H NMR (400 MHz, DMSO-d6) δ 3.29 (t, J=6.5 Hz, 2H), 3.20 (s, 3H), 1.51-1.43 (m, 2H), 2.68 (p, J=6.2 Hz, 2H), 1.37-1.21 (m, 6H)

### 8,8,8-trifluorooctan-1-amine (Compound 4.5, Scheme 4)

Titled compound was synthesized following the general procedure J previously described, using intermediate 4.3 (393 mg, 1.24 mmol) and hydrazine hydrate (0.14 ml, 1.86 mmol) in absolute ethanol (5.5 ml). Purification by basic alumina flash chromatography (dichloromethane/methanol 95:5) afforded the pure title compound (136.31 mg, yield 60%). Characterization: Rt=1.59 min; MS (ESI) m/z: 184.4 [M−H]+. [M−H]− calculated: 183.1. 1H NMR (400 MHz, DMSO-d6) δ 2.78-2.68 (m, 2H), 2.30-2.15 (m, 2H), 1.61-1.41 (m, 4H), 1.38-1.21 (m, 6H).

### General Procedure K for the Synthesis of Compounds 5.2-5.4 (Scheme 5)

4-Fluoro-3-chlorosulfonyl-benzoic acid 3.1 (1 mmol) solved in 2 mL of THF was added dropwise to 8 mL of an ice cold solution of the proper cyclic amine (3 mmol) in THF and stirred for 1 hr at RT. At reaction completion the reaction mixture was evaporated to dryness and the residue treated with water and HCl. The precipitated product was filtered and rinsed with water to afford the pure titled compounds.

### 4-fluoro-3-pyrrolidin-1-ylsulfonyl-benzoic acid (Compound 5.2, Scheme 5)

Title compound was synthesized following the general procedure K previously described using intermediate 3.1 (250 mg, 1.04 mmol) and pyrrolidine (0.26 ml, 3.11 mmol) in THF (8 ml). The described workup afforded pure titled compound (243.2 mg, yield 85%). Characterization: Rt=1.17 min; MS (ESI) m/z: 272.4 [M−H]−. [M−H]− calculated: 273.05. 1H NMR (400 MHz, DMSO-d6) δ 8.30 (dd, J=6.8, 2.3 Hz, 1H), 8.25 (ddd, J=8.6, 4.8, 2.3 Hz, 1H), 7.62 (dd, J=10.1, 8.6 Hz, 1H), 3.28-3.21 (m, 4H), 1.81-1.73 (m, 4H).

### 4-fluoro-3-(1-piperidylsulfonyl)benzoic acid (Compound 5.3, Scheme 5)

Title compound was synthesized following the general procedure K previously described using intermediate 3.1 (250 mg, 1.04 mmol) and pyperidine (0.31 ml, 3.11 mmol) in THF (8 ml). The described workup afforded pure titled compound (257.3 mg, yield 86%). Characterization: Rt=1.34 min; MS (ESI) m/z: 286.4 [M−H]−. [M−H]− calculated: 287.06. 1H NMR (400 MHz, DMSO-d6) δ 8.28-8.23 (m, 2H), 7.65-7.58 (m, 1H), 3.08 (t, J=5.4 Hz, 4H), 1.58-1.49 (m, 4H), 1.46-1.39 (m, 2H).

### 4-fluoro-3-morpholinosulfonyl-benzoic acid (Compound 5.4, Scheme 5)

Title compound was synthesized following the general procedure K previously described using intermediate 3.1 (250 mg, 1.04 mmol) and morpholine (0.27 ml, 3.11 mmol) in THF (8 ml). The described workup afforded pure titled compound (248.1 mg, yield 83%). Characterization: Rt=1.03 min; MS (ESI) m/z: 288.4 [M−H]−. [M−H]− calculated: 289.04. 1H NMR (400 MHz, DMSO-d6) δ 8.32-8.24 (m, 2H), 7.64 (dd, J=10.1, 8.5 Hz, 1H), 3.67-3.60 (m, 4H), 3.10-3.04 (m, 4H).

### 3-pyrrolidin-1-ylsulfonyl-4-(8,8,8-trifluorooctylamino) benzoic acid (Compound 5.5, Scheme 5)

Titled compound was synthesized following the general procedure H previously described using intermediate 5.2 (50 mg, 0.17 mmol) and intermediate 4.5 (34.8 mg, 0.19 mmol) in dry 1,4-Dioxane (0.55 ml). Trituration with diethyl ether (1 ml) afforded the pure titled compound (17.3 mg, yield 23%). Characterization: Rt=2.30 min; MS (ESI) m/z: 435.5 [M−H]−. [M−H]− calculated: 436.2. 1H NMR (400 MHz, DMSO-d6) δ 8.11 (d, J=2.1 Hz, 1H), 7.92 (dd, J=8.8, 2.1 Hz, 1H), 6.89 (d, J=8.9 Hz, 1H), 6.74 (t, J=5.3 Hz, 1H), 3.24 (q, J=6.7 Hz, 2H), 3.18-3.11 (m, 4H), 2.29-2.14 (m, 2H), 1.79-1.68 (m, 4H), 1.57 (m, 2H), 1.46 (m=, 2H), 1.33 (s, 6H).

### 3-(1-piperidylsulfonyl)-4-(8,8,8-trifluorooctylamino) benzoic acid (Compound 5.6, Scheme 5)

Titled compound was synthesized following the general procedure H previously described using intermediate 5.3 (50 mg, 0.17 mmol) and intermediate 4.5 (34.8 mg, 0.19 mmol) in dry 1,4-Dioxane (0.55 ml). Trituration with diethyl ether (1 ml) afforded the pure titled compound (13 mg, yield 17%). Characterization: Rt=2.40 min; MS (ESI) m/z: 449.5 [M−H]−. [M−H]− calculated: 450.2. 1H NMR (400 MHz, DMSO-d6) δ 8.04 (d, J=2.1 Hz, 1H), 7.92 (dd, J=8.8, 2.1 Hz, 1H), 6.89 (d, J=9.0 Hz, 1H), 6.69 (t, J=5.4 Hz, 1H), 3.24 (q, J=6.7 Hz, 2H), 2.98 (t, J=5.4 Hz, 4H), 2.29-2.15 (m, 2H), 1.62-1.55 (m, 2H), 1.55-1.43 (m, 6H), 1.42-1.37 (m, 2H), 1.37-1.30 (m, 6H).

### 3-morpholinosulfonyl-4-(8,8,8-trifluorooctylamino) benzoic acid (Compound 5.7, Scheme 5)

Titled compound was synthesized following the general procedure H previously described using intermediate 5.4 (50 mg, 0.17 mmol) and intermediate 4.5 (34.8 mg, 0.19 mmol) in dry 1,4-Dioxane (0.55 ml). Trituration with diethyl ether (1 ml) afforded the pure titled compound (28.4 mg, yield 37%). Characterization: Rt=2.21 min; MS (ESI) m/z: 451.2 [M−H]−. [M−H]− calculated: 452.16. 1H NMR (400 MHz, Chloroform-d) δ 8.33 (d, J=2.1 Hz, 1H), 8.07 (dd, J=8.9, 2.1 Hz, 1H), 6.87 (t, J=5.0 Hz, 1H), 6.74 (d, J=9.0 Hz, 1H), 3.77-3.70 (m, 4H), 3.21 (q, J=7.0 Hz, 2H), 3.12-3.06 (m, 4H), 2.14-1.99 (m, 2H), 1.73-1.63 (m, 2H), 1.61-1.50 (m, 2H), 1.48-1.32 (m, 6H).

### 5-cyano-2-fluoro-N,N-dimethyl-benzenesulfonamide (Compound 6.2, Reaction L, Scheme 6)

5-cyano-2-fluorobenzene-1-sulfonyl chloride 6.1 (300 mg, 1.35 mmol) solved in 3.5 mL of THF was added dropwise to an ice cold solution of 2 M dimethylamine in THF (0.74 ml, 1.49 mmol) and N,N-diisopropylethylamine (0.48 ml, 2.70 mmol) in 10 ml of THF and then stirred for 30 minutes at rt. At reaction completion the reaction mixture was evaporated to dryness and the residue was portioned between Ethyl acetate (50 ml) and water (50 ml) and the layers were separated. The organic layer was dried over Na₂SO₄ and concentrated to dryness at low pressure. Purification by silica gel flash chromatography (cyclohexane/DCM+1% EtOAc 70:30 to 30:70) afforded the pure title compound (194.2 mg, yield 63%). Characterization: 1H NMR (400 MHz, Chloroform-d) δ 8.20 (dd, J=6.2, 2.2 Hz, 1H), 7.87 (ddd, J=8.6, 4.4, 2.2 Hz, 1H), 7.36 (t, J=8.9 Hz, 1H), 2.89 (d, J=1.9 Hz, 6H).

### 5-cyano-N,N-dimethyl-2-(8,8,8-trifluorooctylamino) benzenesulfonamide (Compound 6.3, Scheme 6)

Titled compound was synthesized following the general procedure H previously described using intermediate 6.2 (194 mg, 0.84 mmol) and intermediate 4.5 (311.5 mg, 1.64 mmol) in dry 1,4-Dioxane (4.2 ml). Trituration with diethyl ether (3 ml) afforded the pure titled compound (317.2 mg, yield 97%). Characterization: Rt=1.82 min; MS (ESI) m/z: 390.3 [M−H]−. [M−H]− calculated: 391.15. 1H NMR (400 MHz, Chloroform-d) δ 7.87 (d, J=2.0 Hz, 1H), 7.57 (dd, J=8.8, 2.1 Hz, 1H), 6.85 (s, 1H), 6.72 (d, J=8.8 Hz, 1H), 3.23-3.13 (m, 2H), 2.77 (s, 6H), 2.14-1.98 (m, 2H), 1.73-1.61 (m, 2H), 1.60-1.48 (m, 4H), 1.46-1.33 (m, 6H).

### 4-fluoro-2-hydroxy-5-sulfamoyl-benzoic acid (Compound 7.3, Reaction M, Scheme 7)

4-fluoro-2-hydroxy-benzoic acid 7.1 (2 g, 12.81 mmol) was stirred in chlorosulfonic acid (4.30 ml, 64.06 mmol) at 120° C. for 4 hr. At reaction completion, the mixture was slowly poured onto ice-cold water (50 ml) and the resulting precipitated solid was collected by filtration to afford intermediate 7.2. This intermediate (1.12 g, 4.35 mmol) was rapidly solved in 10 ml of THF and added to an ice-cold solution of and 0.83 ml of 20% aqueous NH₄OH (4.35 mmol) and trimethylamine (0.61 ml, 4.34 mmol) in 30 ml tetrahydrofuran. The reaction mixture was stirred at 0° C. for 8 hours. After reaction completion the mixture was evaporated to dryness at low pressure and the residue was treated with a saturated NH₄Cl aqueous solution (50 ml) and extracted twice with EtOAc (2×50 ml). The combined organic layers were dried over Na₂SO₄ and concentrated to dryness at low pressure to afford pure titled compound (915.9 mg, yield over two steps 30%). Characterization: Rt=1.15 min; MS (ESI) m/z: 234.3 [M−H]−. [M−H]− calculated: 235. 1H NMR (400 MHz, DMSO-d6) δ 8.21 (d, J=8.5 Hz, 1H), 7.61 (s, 2H), 7.03 (d, J=11.7 Hz, 1H).

### 2-hydroxy-5-sulfamoyl-4-(8,8,8-trifluorooctylamino) benzoic acid (Compound 7.4, Scheme 7)

Titled compound was synthesized following the general procedure H previously described using intermediate 7.3 (250 mg, 1.02 mmol) and intermediate 4.5 (377.7 mg, 2.04 mmol) in dry 1,4-Dioxane (3.4 ml). Trituration with cyclohexane (3 ml) afforded the pure titled compound (286 mg, yield 69%). Characterization: Rt=1.81 min; MS (ESI) m/z: 397.3 [M−H]−. [M−H]− calculated: 398.1. 1H NMR (400 MHz, DMSO-d6) δ 8.10 (s, 1H), 7.32 (s, 2H), 6.36 (t, J=5.3 Hz, 1H), 6.12 (s, 1H), 3.18 (q, J=6.8 Hz, 2H), 2.29-2.15 (m, 2H), 1.64-1.54 (m, 2H), 1.52-1.42 (m, 2H), 1.41-1.29 (m, 6H).

### tert-butyl 4-(5,5,5-trifluoropentyl)piperazine-1-carboxylate (Compound 8.2, Reaction N, Scheme 8)

To a solution of 1-boc-piperazine 8.1 (400 mg, 2.15 mmol) in acetonitrile (5 mL) cooled at 0° C. were added 5-iodo-1, 1,1-trifluoropentane (0.25 mL, 3.22 mmol) and N,N-diisopropylethylamine (0.57 mL, 3.22 mmol) and the reaction mixture was stirred at room temperature for 24 hours. At reaction completion the reaction crude was concentrated to dryness at low pressure. The residue was solved EtOAc (25 mL) and washed with water (25 mL) and Brine (25 mL). The organic layer was dried over Na₂SO₄ and concentrated to dryness at low pressure. Purification by silica gel flash chromatography (dichloromethane/methanol 98:2) afforded the pure title compound (378.9 mg, yield 92%). Characterization: Rt=2.02; MS (ESI) m/z: 311.5 [M−H]+. [M−H]− calculated: 310.2. 1H NMR (400 MHz, Chloroform-d) δ 3.42 (t, J=4.7 Hz, 4H), 2.41-2.31 (m, 6H), 2.16-2.02 (m, 2H), 1.63-1.50 (m, 4H), 1.45 (s, 9H).

### 1-(5,5,5-trifluoropentyl)piperazine di-trifluoroacetate (Compound 8.3, Reaction O, Scheme 8)

Intermediate 8.2 (378.9 mg, 2.01 mmol) was stirred in neat trifluoroacetic acid (1.5 mL) at room temperature for 1.5 hours. At reaction completion, the reaction crude was diluted with DCM and concentrated to dryness at low pressure three times (3×10 ml) and once with MeOH (10 ml) to afford the pure titled compound (717.5 mg, yield 81%). Characterization: 1H NMR (400 MHz, Methanol-d4) δ 3.59-3.48 (m, 8H), 3.31-3.28 (m, 2H), 3.22-3.15 (m, 2H), 2.30-2.17 (m, 2H), 1.87-1.78 (m, 2H), 1.68-1.59 (m, 2H).

### 3-(dimethylsulfamoyl)-4-[4-(5,5,5 trifluoropentyl)piperazin-1-yl] benzoic acid (Compound 9.1, Reaction P, Scheme 9)

Under Argon atmosphere, to a solution of intermediate 8.3 (106.4 mg, 0.24 mmol) and triethylamine (0.14 ml, 1.00 mmol) in dry 1,4-dioxane (1 ml) was added intermediate 3.3 (50 mg, 0.20 mmol) solved in 1,4-dioxane (1 ml) and the reaction mixture was stirred at 100° C. for 24 hours. At reaction completion, the reaction crude was portioned between ethyl acetate (25 ml) and a saturated NH₄Cl solution (25 ml) and pH was adjusted to 3 with concentrated HCl. The Layers were separated and the aqueous layer was washed with diethyl ether (25 ml). The aqueous layer was then neutralized to pH 7 and extracted with ethyl acetate (3×25 ml) and with DCM (25 ml). The combined organic layers were dried over Na₂SO₄ and concentrated to dryness at low pressure. Trituration with diethyl ether (2 ml) afforded the pure title compound (26.7 mg, yield 30%). Characterization: Rt=1.31; MS (ESI) m/z: 436.5 [M−H]−. [M−H]− calculated: 437.2. 1H NMR (400 MHz, DMSO-d6) δ 8.33 (d, J=2.1 Hz, 1H), 8.12 (dd, J=8.3, 2.2 Hz, 1H), 7.56 (d, J=8.4 Hz, 1H), 3.08-2.99 (m, 4H), 2.67 (s, 6H), 2.57-2.53 (m, 4H), 2.40-2.34 (m, 2H), 2.34-2.18 (m, 2H), 1.58-1.46 (m, 4H).

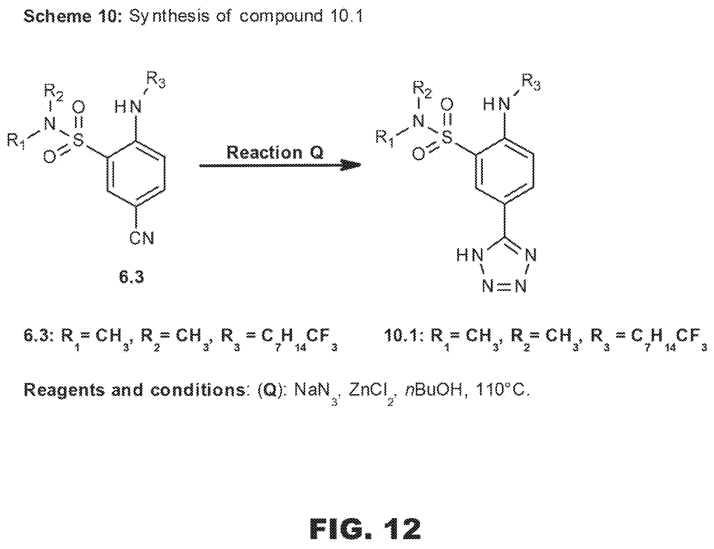

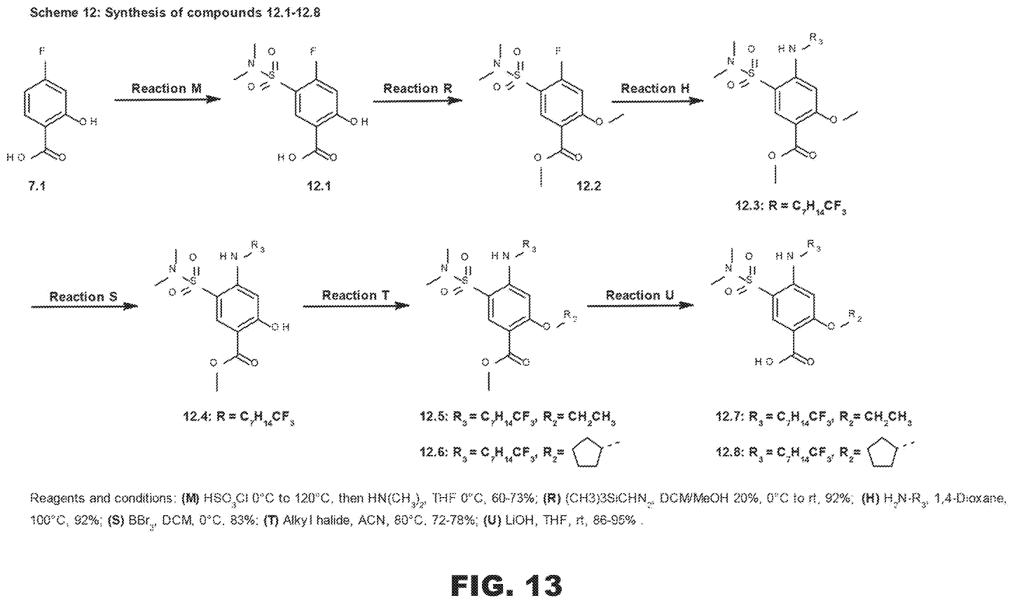

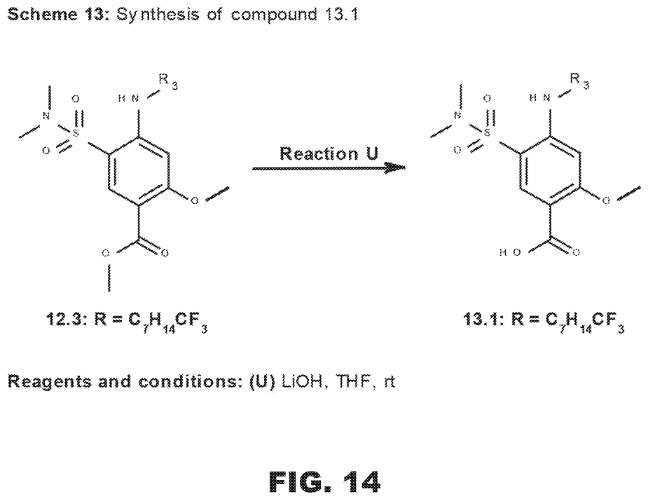

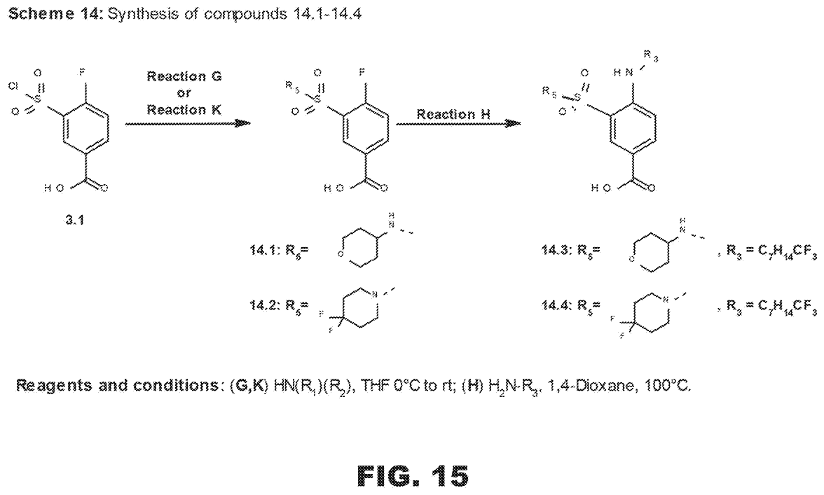

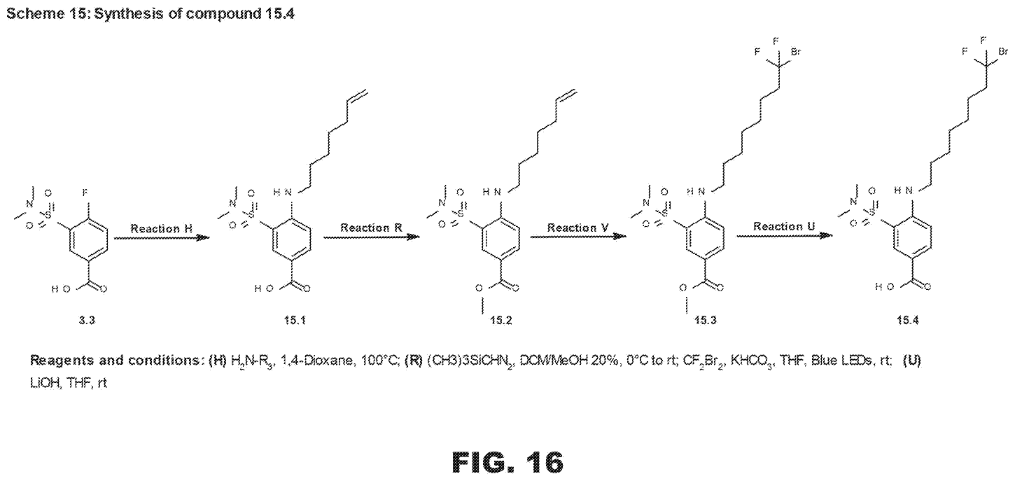

### N,N-dimethyl-5-(1H-tetrazol-5-yl)-2 (8,8,8-trifluorooctylamino)benzenesulfonamide (Compound 10.1 Scheme 10, FIG. 12)

A mixture of intermediate 6.3 (317.2 mg, 0.8 mmol), sodium azide (63.2 mg, 0.96 mmol) and zinc chloride (132.6 mg, 0.96 mmol) was stirred in 4 ml of n-butanol at 110° C. for 10 hours. At reaction completion the reaction mixture was evaporated to dryness at low pressure. Next, 5% NaOH (20 mL) was added and the mixture was stirred for 20 min. The resulting suspension was filtered, and the solid washed with 5% NaOH (10 mL). The pH of the filtrate was adjusted to 1.0 with concentrated HCl and was extracted 3 times with EtOAc (3×25 ml). The combined organic layers were dried over Na₂SO₄ and concentrated to dryness at low pressure. Purification by silica gel flash chromatography (dichloromethane/methanol 98:2) finally afforded the pure titled compound (110.93 mg, yield 32%). Characterization: Rt=0.77; MS (ESI) m/z: 433.3 [M−H]−. [M−H]− calculated: 434.2. 1H NMR (400 MHz, Chloroform-d) δ 8.25 (d, J=2.1 Hz, 1H), 8.19 (dd, J=8.8, 2.2 Hz, 1H), 6.85 (d, J=8.9 Hz, 1H), 6.61 (s, 1H), 3.19 (t, J=7.1 Hz, 2H), 2.76 (s, 6H), 2.14-1.98 (m, 2H), 1.73-1.62 (m, 2H), 1.61-1.49 (m, 2H), 1.49-1.30 (m, 6H).

### 5-(N,N-dimethylsulfamoyl)-4-fluoro-2-hydroxybenzoic acid (Compound 12.1, Scheme 12)

4-fluoro-2-hydroxy-benzoic acid 7.1 (2 g, 12.81 mmol) was stirred in chlorosulfonic acid (4.30 ml, 64.06 mmol) at 120° C. for 4 hr. At reaction completion, the mixture was slowly poured onto ice-cold water (50 ml) and the resulting precipitated solid was collected by filtration. The collected solid (1.141 g) was solved in 10 ml of THF and added dropwise to an ice-cold solution of 2M dimethylamine in THF (3 ml,) and DIPEA (3 ml) in 35 ml tetrahydrofuran. The reaction mixture was stirred at 0° C. for 8 hours. At reaction completion the mixture was evaporated to dryness at low pressure and the residue was treated with a saturated NH₄Cl aqueous solution (50 ml) and extracted twice with EtOAc (2×50 ml). The combined organic layers were dried over Na₂SO₄ and concentrated to dryness at low pressure to afford pure titled compound (823.9 mg, 70% yield). UPLC/MS: Rt=1.19 min (gradient 1); MS (ESI) m/z: 262.0 [M−H]⁻. [M−H]⁻ calculated: 262.0. ¹H NMR (400 MHz, DMSO-d₆) δ 8.15 (d, J=8.2 Hz, 1H), 7.13-7.03 (m, 1H), 2.71 (d, J=1.7 Hz, 6H).

### Methyl 5-(N,N-dimethylsulfamoyl)-4-fluoro-2-methoxybenzoate (Compound 12.2, Scheme 12)

To an ice cold solution of intermediate 12.1 (200 mg, 0.75 mmol) in DCM/MeOH 8:2 (9 ml) was carefully added trimethylsilyldiazomethane (2M in hexanes, 1.13 ml, 2.26 mmol) and the reaction mixture was stirred at room temperature for 2 hours. At reaction completion the reaction mixture was quenched with 2 ml of a 1M acetic solution in methanol and evaporated to dryness. The dry residue was suspended in a saturated NaHCO₃ (15 ml) aqueous solution and extracted twice with EtOAc (2×15 ml). Purification by silica gel flash chromatography (cyclohexane/EtOAc from 85:15 to 70:30) afforded the pure titled compound (201 mg, 92% yield) as a white solid. UPLC/MS: Rt=1.75 min (gradient 1); MS (ESI) m/z: 292.1 [M+H]⁺. [M+H]⁺ calculated: 292.0. ¹H NMR (600 MHz, Chloroform-d) δ 8.35 (d, J=5.0 Hz, 1H), 6.94 (d, J=8.0 Hz, 1H), 3.85 (s, 3H), 3.79 (s, 3H), 2.72 (s, 6H).

### Methyl 5-(N,N-dimethylsulfamoyl)-2-methoxy-4-((8,8,8-trifluorooctyl)amino)benzoate (Compound 12.3, Scheme 12)

Compound 12.3 was synthesized following the general procedure H previously described using intermediate 12.2 (50 mg, 0.17 mmol) and intermediate 4.5 (75.4 mg, 0.34 mmol) in dry 1,4-dioxane (0.85 ml). Purification by silica gel flash chromatography (cyclohexane/EtOAc from 80:15 to 75:25) afforded the pure titled compound (64.9 mg, 84% yield) as a white solid. UPLC/MS: Rt=2.65 min (gradient 1); MS (ESI) m/z: 455.3 [M+H]⁺. [M+H]⁺ calculated: 455.2. ¹H NMR (400 MHz, Chloroform-d) δ 8.23 (s, 1H), 6.77 (t, J=4.8 Hz, 1H), 6.10 (s, 1H), 3.97 (s, 3H), 3.84 (s, 3H), 3.22-3.16 (m, 2H), 2.75 (s, 6H), 2.14-2.04 (m, 2H), 1.72 (p, J=7.1 Hz, 2H), 1.60-1.55 (m, 4H), 1.45 (dd, J=5.0, 2.0 Hz, 2H), 1.41 (dd, J=3.9, 2.6 Hz, 4H).

### Methyl 5-(N,N-dimethylsulfamoyl)-2-hydroxy-4-((8, 8, 8-trifluorooctyl)amino)benzoate (Compound 12.4, Scheme 12)

Under argon atmosphere, to an ice cold solution of intermediate 12.3 (50 mg, 0.11 mmol) solved in DCM (1.2 mL) was added dropwise BBr₃ (1 M in DCM, 0.55 ml, 0.55 mmol) and the mixture was stirred at room temperature for 6 hours. At reaction completion, the reaction mixture was cooled to 0° C., quenched with 2 ml of methanol and evaporated to dryness. The dry residue crude was then portioned between EtOAc (10 ml) and an NH₄Cl saturated solution (10 ml) and the layers separated. The organic layer was dried over Na₂SO₄ and concentrated to dryness at low pressure. Purification by silica gel flash chromatography (cyclohexane/EtOAc 95:05) afforded the pure titled compound (40.2 mg, 83% yield) as a white solid. UPLC/MS: Rt=2.10 min (gradient 1); MS (ESI) m/z: 441.3 [M−H]⁺. [M+H]⁺ calculated: 441.1. ¹H NMR (400 MHz, chloroform-d) δ 11.26 (s, 1H), 8.17 (s, 1H), 6.73 (t, J=4.6 Hz, 1H), 6.16 (s, 1H), 3.92 (s, 3H), 3.16 (q, J=7.1, 5.0 Hz, 2H), 2.75 (s, 6H), 2.15-1.99 (m, 2H), 1.74-1.63 (m, 2H), 1.62-1.54 (m, 2H), 1.48-1.35 (m, 6H).

### Methyl 5-(N,N-dimethylsulfamoyl)-2-ethoxy-4-((8,8,8-trifluorooctyl)amino)benzoate (Compound 12.5, Scheme 12)

To a solution of intermediate 12.4 (31.8 mg, 0.07 mmol) in acetonitrile (0.7 mL) were added ethyl iodide (10 μl, 0.11 mmol) and potassium carbonate (15 mg, 0.11 mmol) and the reaction mixture was stirred at 80° C. for hours. At reaction completion, the crude was portioned between EtOAc (10 ml) and water (10 ml) and the layers separated. The organic layer was dried over Na₂SO₄ and concentrated to dryness at low pressure. Purification by silica gel flash chromatography (cyclohexane/EtOAc from 100:00 to 80:20) afforded the pure titled compound (25.6 mg, 78% yield) as a white solid. UPLC/MS: Rt=1.85 min (gradient 1); MS (ESI) m/z: 469.3 [M+H]⁺. [M+H]⁺ calculated: 469.2. ¹H NMR (400 MHz, Chloroform-d) δ 8.20 (s, 1H), 6.71 (t, J=4.8 Hz, 1H), 6.07 (s, 1H), 4.14 (q, J=7.0 Hz, 2H), 3.82 (s, 3H), 3.18-3.11 (m, 2H), 2.72 (s, 6H), 2.13-1.99 (m, 2H), 1.73-1.64 (m, 2H), 1.61-1.53 (m, 2H), 1.51 (t, J=6.9 Hz, 3H), 1.48-1.35 (m, 6H).

### 5-(N,N-dimethylsulfamoyl)-2-ethoxy-4-((8,8,8-trifluorooctyl)amino)benzoic acid (Compound 12.7, Scheme 12)

To a solution of compound 12.5 (25.6 mg, 0.05 mmol) in tetrahydrofuran (0.5 mL) was added a 1 M LiOH aqueous solution (0.27 ml, 0.27 mmol) and the reaction mixture was stirred at room temperature for 16 hr. At reaction completion, the crude was portioned between EtOAc (10 ml) and an NH₄Cl saturated solution (10 ml) and the layers separated. The organic layer was dried over Na₂SO₄ and concentrated to dryness at low pressure. Trituration with cyclohexane afforded the pure titled compound (19.54 mg, 86% yield) as a white solid. UPLC/MS: Rt=1.32 min (gradient 1); MS (ESI) m/z: 453.3 [M−H]⁻. [M−H]⁻ calculated: 453.2. ¹H NMR (400 MHz, DMSO-d₆) δ 7.95 (s, 1H), 6.62 (t, J=5.2 Hz, 1H), 6.23 (s, 1H), 4.15 (q, J=6.9 Hz, 2H), 3.23 (q, J=6.5 Hz, 2H), 2.60 (s, 6H), 2.29-2.14 (m, 2H), 1.63-1.52 (m, 2H), 1.51-1.42 (m, 2H), 1.40-1.25 (m, 9H).

### Methyl 2-(cyclopentyloxy)-5-(N,N-dimethylsulfamoyl)-4-((8,8,8-trifluorooctyl)amino)benzoate (Compound 12.6, Scheme 12)

To a solution of intermediate 12.4 (30.0 mg, 0.07 mmol) in acetonitrile (0.7 mL) were added cyclopentyl bromide (15 μl, 0.13 mmol) and potassium carbonate (28.3 mg, 0.20 mmol) and the reaction mixture was stirred at 80° C. for 4 hours. At reaction completion, the crude was portioned between EtOAc (10 ml) and water (10 ml) and the layers separated. The organic layer was dried over Na₂SO₄ and concentrated to dryness at low pressure. Purification by silica gel flash chromatography (cyclohexane/EtOAc from 100:00 to 90:10) afforded the pure titled compound (25.6 mg, 72% yield) as a white solid. UPLC/MS: Rt=2.30 min (gradient 2); MS (ESI) m/z: 509.2 [M+H]⁺. [M+H]⁺ calculated: 509.6. ¹H NMR (400 MHz, Chloroform-d) δ 8.19 (s, 1H), 6.69 (t, J=4.8 Hz, 1H), 6.07 (s, 1H), 4.88-4.81 (m, 1H), 3.80 (s, 3H), 3.19-3.10 (m, 2H), 2.72 (s, 6H), 2.13-1.99 (m, 2H), 1.99-1.92 (m, 4H), 1.91-1.81 (m, 2H), 1.73-1.62 (m, 2H), 1.61-1.51 (m, 2H), 1.49-1.34 (m, 6H).

### 2-(cyclopentyloxy)-5-(N,N-dimethylsulfamoyl)-4-((8,8,8-trifluorooctyl)amino)benzoic acid (Compound 12.8, Scheme 12)

To a solution of intermediate 12.6 (25.6 mg, 0.05 mmol) solved in tetrahydrofuran (0.25 mL) was added a 1 M LiOH aqueous solution (0.5 ml, 0.25 mmol) and the mixture was stirred at room temperature for 16 hours. At reaction completion, the crude was portioned between EtOAc (10 ml) and an NH₄Cl saturated solution (10 ml) and the layers separated. The organic layer was dried over Na₂SO₄ and concentrated to dryness at low pressure. Trituration with cyclohexane afforded the pure titled compound (16.3 mg, 66% yield) as a white solid. UPLC/MS: Rt=1.80 min (gradient 1); MS (ESI) m/z: 493.3 [M−H]⁻. [M−H]⁻ calculated: 493.2. ¹H NMR (400 MHz, Chloroform-d) ¹H NMR (400 MHz, Chloroform-d) δ 8.40 (s, 1H), 6.94 (s, 1H), 6.12 (s, 1H), 5.09-5.03 (m, 1H), 3.20-3.13 (m, 2H), 2.75 (s, 6H), 2.14-1.97 (m, 5H), 1.93-1.81 (m, 2H), 1.81-1.65 (m, 4H), 1.61-1.51 (m, 4H), 1.50-1.33 (m, 6H).

### 5-(N,N-dimethylsulfamoyl)-2-methoxy-4-((8,8,8-trifluorooctyl)amino)benzoic acid (Compound 13.1, Scheme 13)

To a solution of intermediate 12.3 (59 mg, 0.13 mmol) solved in tetrahydrofuran (1.3 mL) was added a 1 M LiOH aqueous solution (0.26 ml, 0.26 mmol) and the mixture was stirred at room temperature for 16 hours. At reaction completion, the crude was portioned between EtOAc (10 ml) and an NH₄Cl saturated solution (10 ml) and the layers separated. The organic layer was dried over Na₂SO₄ and concentrated to dryness at low pressure. Trituration with cyclohexane afforded the pure titled compound (41.2 mg, 72% yield) as a white solid. UPLC/MS: Rt=1.16 min (gradient 1); MS (ESI) m/z: 439.5 [M−H]⁻. [M−H]⁻ calculated: 439.2. ¹H NMR (400 MHz, DMSO-d₆) δ 7.98 (s, 1H), 6.65 (t, J=5.2 Hz, 1H), 6.26 (s, 1H), 3.88 (s, 3H), 3.29-3.22 (m, 2H), 2.61 (s, 6H), 1.65-1.55 (m, 2H), 1.52-1.42 (m, 4H), 1.39-1.29 (m, 6H).

### 4-fluoro-3-(N-(tetrahydro-2H-pyran-4-yl)sulfamoyl)benzoic acid (Compound 14.1, Scheme 14)

Title compound was synthesized following the general procedure G previously described using intermediate 3.1 (250 mg, 1.04 mmol) and tetrahydro-2H-pyran-4-amine (0.32 ml, 2.07 mmol) in THF (8.5 ml). The described workup afforded the pure titled compound (160.9 mg, 51% yield) as a white solid. UPLC/MS: Rt=0.93 min (gradient 1); MS (ESI) m/z: 302.1 [M−H]⁻. [M−H] calculated: 302.06. ¹H NMR (400 MHz, DMSO-d₆) δ 8.34 (dd, J=7.1, 2.3 Hz, 1H), 8.27 (d, J=7.8 Hz, 1H), 8.24-8.18 (m, 1H), 7.57 (t, J=9.3 Hz, 1H), 3.77-3.68 (m, 2H), 3.27-3.19 (m, 3H), 1.58-1.49 (m, 2H), 1.49-1.37 (m, 2H).

### 3-((4,4-difluoropiperidin-1-yl)sulfonyl)-4-fluorobenzoic acid (Compound 14.2, Scheme 14)

Title compound was synthesized following the general procedure K previously described using intermediate 3.1 (150 mg, 0.62 mmol) and 4,4-difluoropiperidine hydrochloride (198.1 mg, 1.24 mmol) and DIPEA (0.33 ml, 1.87 mmol) in THF (5.0 ml). At reaction completion the reaction mixture was evaporated to dryness. The described workup afforded the pure titled compound (176.4 mg, 88% yield) as a white solid. UPLC/MS: Rt=1.38 min (gradient 1); MS (ESI) m/z: 322.0 [M−H]⁻. [M−H] calculated: 322.04. ¹H NMR (400 MHz, DMSO-d₆) δ 8.31-8.25 (m, 2H), 7.67-7.60 (m, 1H), 3.29 (t, J=5.8 Hz, 4H), 2.07 (ddd, J=19.7, 13.7, 5.8 Hz, 4H).

### 3-morpholinosulfonyl-4-((8,8,8-trifluorooctyl)amino)benzoic acid (Compound 14.3, Scheme 14)

Titled compound was synthesized following the general procedure H previously described using intermediate 14.2 (50 mg, 0.17 mmol) and intermediate 4.5 (34.8 mg, 0.19 mmol) in dry 1,4-dioxane (0.55 ml). Purification by silica gel flash chromatography (CH₂Cl₂/MeOH from 100:0 to 98:02) followed by trituration with diethyl ether (1 ml) afforded the pure titled compound (28.4 mg, 37% yield) as a white solid. UPLC/MS: Rt=2.21 min (gradient 1); MS (ESI) m/z: 451.2 [M−H]⁻. [M−H]⁻ calculated: 451.2. ¹H NMR (400 MHz, Chloroform-d) δ 8.33 (d, J=2.1 Hz, 1H), 8.07 (dd, J=8.9, 2.1 Hz, 1H), 6.87 (t, J=5.0 Hz, 1H), 6.74 (d, J=9.0 Hz, 1H), 3.77-3.70 (m, 4H), 3.21 (q, J=7.0 Hz, 2H), 3.12-3.06 (m, 4H), 2.14-1.99 (m, 2H), 1.73-1.63 (m, 2H), 1.61-1.50 (m, 2H), 1.48-1.32 (m, 6H).

### 3-((4,4-difluoropiperidin-1-yl)sulfonyl)-4-((8,8,8-trifluorooctyl)amino)benzoic acid (Compound 14.4, Scheme 14)

Titled compound was synthesized following the general procedure H previously described using intermediate 14.1 (50 mg, 0.15 mmol) and intermediate 4.5 (34.8 mg, 0.19 mmol) in dry 1,4-dioxane (0.55 ml). Purification by silica gel flash chromatography (CH₂Cl₂/MeOH from 100:0 to 98:02) followed by trituration with petroleum ether (1 ml) afforded the pure titled compound (22.6 mg, 31% yield) as a white solid. UPLC/MS: Rt=2.39 min (gradient 1); MS (ESI) m/z: 485.2 [M−H]⁻. [M−H]− calculated: 485.2. ¹H NMR (400 MHz, Chloroform-d) δ 8.35 (d, J=2.0 Hz, 1H), 8.08 (dd, J=8.9, 2.1 Hz, 1H), 6.78 (t, J=5.0 Hz, 1H), 6.74 (d, J=9.0 Hz, 1H), 3.31 (t, J=5.8 Hz, 4H), 3.25-3.18 (m, 2H), 2.14-2.00 (m, 6H), 1.69 (p, J=7.0 Hz, 2H), 1.62-1.52 (m, 2H), 1.49-1.35 (m, 6H).

### 3-(dimethylsulfamoyl)-4-(hept-6-enylamino)benzoic acid (Compound 15.1, Scheme 15)

Titled compound was synthesized following the general procedure H previously described using intermediate 3.3 (420 mg, 1.68 mmol) and kept-6-en-1-amine hydrochloride (335.6 mg, 1.68 mmol) in dry 1,4-dioxane (16.5 ml). Purification by silica gel flash chromatography (CH₂Cl₂/MeOH from 100:0 to 98:02) followed by trituration with diethyl ether (3 ml) afforded the pure titled compound (409.6 mg, 72% yield) as a white solid. UPLC/MS: Rt=2.13 min (gradient 1); MS (ESI) m/z: 439.2 [M−H]⁻. [M−H]− calculated: 339.1. ¹H NMR (400 MHz, Chloroform-d) δ 8.34 (d, J=2.0 Hz, 1H), 8.06 (dd, J=8.9, 2.1 Hz, 1H), 6.91 (t, J=5.0 Hz, 1H), 6.72 (d, J=9.0 Hz, 1H), 5.80 (ddt, J=16.9, 10.2, 6.7 Hz, 1H), 5.04-4.91 (m, 2H), 3.24-3.18 (m, 2H), 2.77 (s, 6H), 2.13-2.02 (m, 2H), 1.69 (p, J=7.0 Hz, 2H), 1.49-1.39 (m, 4H).

### Methyl 3-(N,N-dimethylsulfamoyl)-4-(hept-6-en-1-ylamino)benzoate (Compound 15.2, Scheme 12)

To an ice cold solution of intermediate 15.1 (220 mg, 0.64 mmol) in DCM/MeOH 8:2 (8 ml) was carefully added trimethylsilyldiazomethane (2M in hexanes, 0.48 ml, 0.96 mmol) and the reaction mixture was stirred at room temperature for 2 hours. At reaction completion the reaction mixture was quenched with 2 ml of a 1M acetic solution in methanol and evaporated to dryness. The dry residue was suspended in a saturated NaHCO₃ (15 ml) aqueous solution and extracted twice with EtOAc (2×15 ml). Purification by silica gel flash chromatography (cyclohexane/EtOAc from 100:00 to 90:10) afforded the pure titled compound (213.2 mg, 94% yield) as a white solid. UPLC/MS: Rt=1.81 min (gradient 1); MS (ESI) m/z: 355.2 [M+H]⁺. [M+H]⁺ calculated: 355.2. ¹H NMR (600 MHz, Chloroform-d) ¹H NMR (400 MHz, Chloroform-d) δ 8.28 (d, J=2.1 Hz, 1H), 8.01 (dd, J=8.9, 2.1 Hz, 1H), 6.83-6.74 (m, 1H), 6.70 (d, J=8.9 Hz, 1H), 5.79 (ddt, J=16.9, 10.2, 6.7 Hz, 1H), 5.04-4.92 (m, 2H), 3.87 (s, 3H), 3.23-3.15 (m, 2H), 2.75 (s, 6H), 2.12-2.03 (m, 2H), 1.74-1.63 (m, 2H), 1.49-1.38 (m, 4H).

### Methyl 4-((8-bromo-8,8-difluorooctyl)amino)-3-(N,N-dimethylsulfamoyl)benzoate (Compound 15.3, Scheme 15)

In a sealed glass tube, to a solution of intermediate 15.2 (213.2 mg, 0.62 mmol) in THF (6.2 ml) were added potassium bicarbonate (62.7 mg, 0.62 mmol), eosin salt (23.8 mg 0.03 mmol) and dibromodifluoromethane (0.12 ml 1.24 mmol). The reaction mixture was then stirred at room temperature under blue LEDs irradiation (A=460-470 nm) for 16 Nous. At reaction completion the reaction mixture evaporated to dryness. The dry residue was suspended in water (25 ml) aqueous solution and extracted twice with EtOAc (2×25 ml). Purification by silica gel flash chromatography (Petroleum ether/TBME from 100:00 to 80:20) afforded the pure titled compound (144.5 mg, 48% yield) as a white solid. 15). UPLC/MS: Rt=2.13 min (gradient 2); MS (ESI) m/z: 485.0 [M+H]⁺. [M+H]⁺ calculated: 485.08 ¹H NMR (600 MHz, Chloroform-d) ¹H NMR (400 MHz, Chloroform-d) 1H NMR (400 MHz, Chloroform-d) δ 8.27 (d, J=2.1 Hz, 1H), 8.02 (dd, J=8.9, 2.1 Hz, 1H), 6.79 (t, J=5.0 Hz, 1H), 6.70 (d, J=8.9 Hz, 1H), 3.87 (s, 3H), 3.23-3.16 (m, 2H), 2.76 (s, 6H), 2.40-2.26 (m, 2H), 1.72-1.55 (m, 6H), 1.48-1.35 (m, 6H).

### 4-[(8-bromo-8,8-difluoro-octyl)amino]-3-(dimethylsulfamoyl)benzoic acid (Compound 15.4, Scheme 15)

To a solution of intermediate 15.3 (50 mg, 0.10 mmol) solved in tetrahydrofuran (1.0 mL) was added a 1 M LiOH aqueous solution (0.42 ml, 0.2 mmol) and the mixture was stirred at room temperature for 16 hours. At reaction completion, the crude was portioned between EtOAc (10 ml) and an NH₄Cl saturated solution (10 ml) and the layers separated. The organic layer was dried over Na₂SO₄ and concentrated to dryness at low pressure. Trituration with cyclohexane afforded the pure titled compound (40.1 mg, 85% yield) as a white solid. UPLC/MS: Rt=1.22 min (gradient 2); MS (ESI) m/z: 469.1 [M−H]⁻. [M−H]⁻ calculated: 469.1. ¹H NMR (400 MHz, Chloroform-d) 1H NMR (400 MHz, Chloroform-d) δ 8.29 (d, J=2.1 Hz, 1H), 8.05 (dd, J=8.9, 2.1 Hz, 1H), 6.83 (t, J=5.0 Hz, 1H), 6.70 (d, J=8.9 Hz, 1H), 3.25-3.18 (m, 2H), 2.77 (s, 6H), 2.42-2.28 (m, 2H), 1.76-1.59 (m, 6H), 1.51-1.38 (m, 6H).

### Example 2: Activity Data

The data obtained are reported in table 1 below.

TABLE 1

| | | | | NKCC1 |
|---|---|---|---|---|
| | | | | Inhibition |
| | | | NKCC1 | % 10 μM |
| | | | Inhibition | Neurons |
| | | | % 10 μM HEK | Calcium |
| | | | Chloride YFP | kinetic |
| Entry | Structure | Compound id | assay | assay |
| 1 | 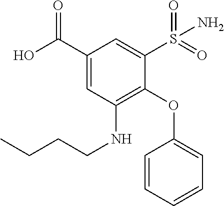 [SMILES: CCCCNc1cc(C(=O)O)cc(S(N)(=O)=O)c1Oc1ccccc1] | Bumetanide | 54% | 52% |
| 2 | 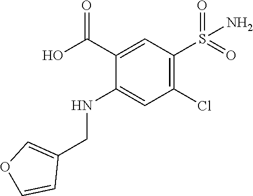 [SMILES: NS(=O)(=O)c1cc(C(=O)O)c(NCc2ccoc2)cc1Cl] | Furosemide | Inactive | 36% |
| 3 | 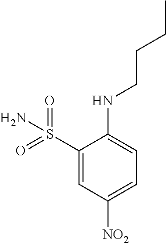 [SMILES: CCCCNc1ccc([N+](=O)[O-])cc1S(N)(=O)=O] | 1.6 | 11% | n.a |
| 4 | 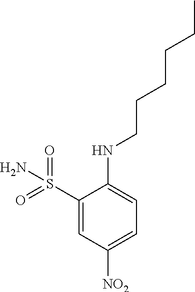 [SMILES: CCCCCCNc1ccc([N+](=O)[O-])cc1S(N)(=O)=O] | 1.7 | 25% | 36% |
| 5 | 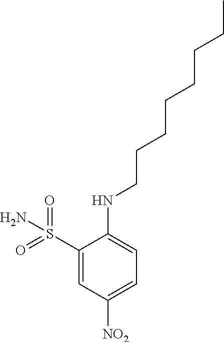 [SMILES: CCCCCCCCNc1ccc([N+](=O)[O-])cc1S(N)(=O)=O] | 1.8 | 12% | n.a. |
| 6 | 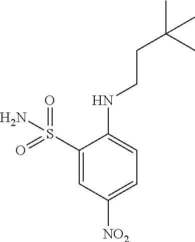 [SMILES: CC(C)(C)CCNc1ccc([N+](=O)[O-])cc1S(N)(=O)=O] | 1.9 | 5% | n. a |
| 7 | 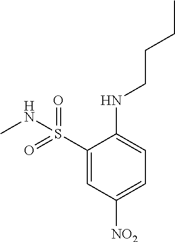 [SMILES: CCCCNc1ccc([N+](=O)[O-])cc1S(=O)(=O)NC] | 1.10 | Inactive | n.a |
| 8 | 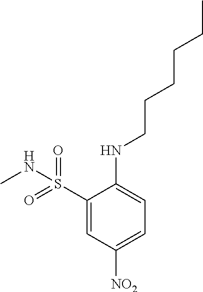 [SMILES: CCCCCCNc1ccc([N+](=O)[O-])cc1S(=O)(=O)NC] | 1.11 | 3, 4% | n.a |
| 9 | 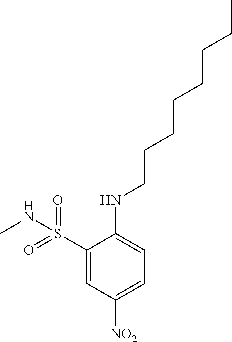 [SMILES: CCCCCCCCNc1ccc([N+](=O)[O-])cc1S(=O)(=O)NC] | 1.12 | Inactive | n.a |
| 10 | 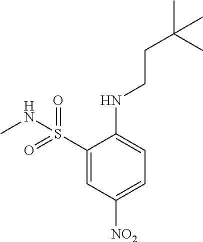 [SMILES: CNS(=O)(=O)c1cc([N+](=O)[O-])ccc1NCCC(C)(C)C] | 1.13 | 17% | n.a |
| 11 | 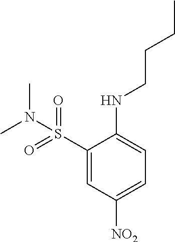 [SMILES: CCCCNc1ccc([N+](=O)[O-])cc1S(=O)(=O)N(C)C] | 1.14 | Inactive | n.a |
| 12 | 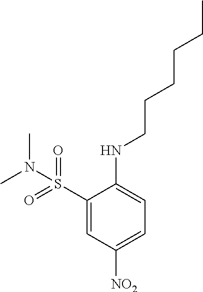 [SMILES: CCCCCCNc1ccc([N+](=O)[O-])cc1S(=O)(=O)N(C)C] | 1.15 | 16% | n.a |
| 13 | 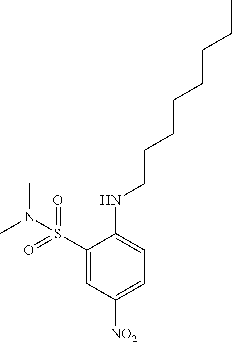 [SMILES: CCCCCCCCNc1ccc([N+](=O)[O-])cc1S(=O)(=O)N(C)C] | 1.16 | Inactive | n.a |
| 14 | 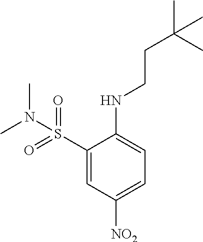 [SMILES: CN(C)S(=O)(=O)c1cc([N+](=O)[O-])ccc1NCCC(C)(C)C] | 1.17 | Inactive | n.a |
| 15 | 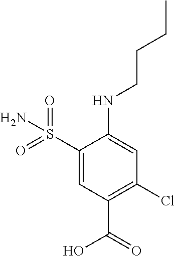 [SMILES: CCCCNc1cc(Cl)c(C(=O)O)cc1S(N)(=O)=O] | 2.2 | 36% | 2% |
| 16 | 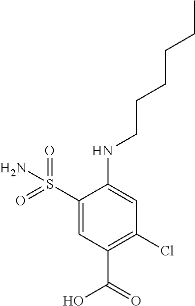 [SMILES: CCCCCCNc1cc(Cl)c(C(=O)O)cc1S(N)(=O)=O] | 2.3 | 10% | n. a |
| 17 | 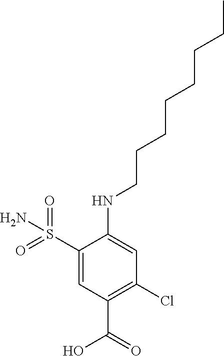 [SMILES: CCCCCCCCNc1cc(Cl)c(C(=O)O)cc1S(N)(=O)=O] | 2.4 | 14% | n. a |
| 18 | 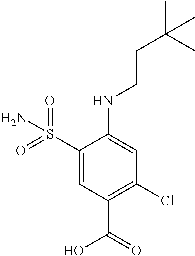 [SMILES: CC(C)(C)CCNc1cc(Cl)c(C(=O)O)cc1S(N)(=O)=O] | 2.5 | Inactive | n.a |
| 19 | 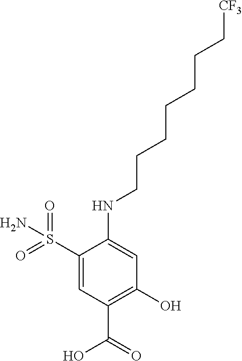 [SMILES: NS(=O)(=O)c1cc(C(=O)O)c(O)cc1NCCCCCCCC(F)(F)F] | 7.4 | 30% | 24% |
| 20 | 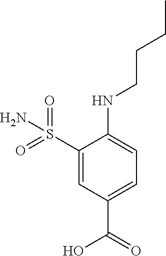 [SMILES: CCCCNc1ccc(C(=O)O)cc1S(N)(=O)=O] | 2.6 | Inactive | 14% |
| 21 | 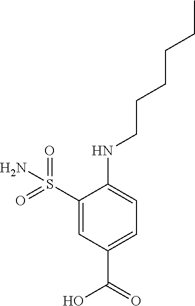 [SMILES: CCCCCCNc1ccc(C(=O)O)cc1S(N)(=O)=O] | 2.7 | 4% | 10% |
| 22 | 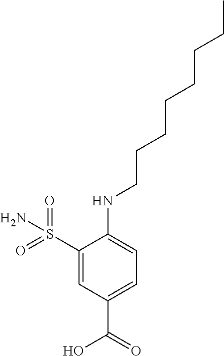 [SMILES: CCCCCCCCNc1ccc(C(=O)O)cc1S(N)(=O)=O] | 2.8 | 5% | 3% |
| 23 | 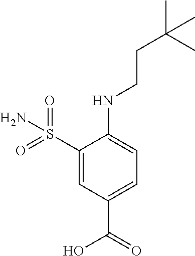 [SMILES: CC(C)(C)CCNc1ccc(C(=O)O)cc1S(N)(=O)=O] | 2.9 | 1% | 25% |
| 24 | 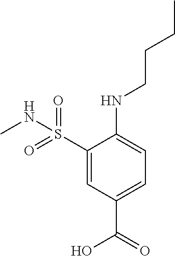 [SMILES: CCCCNc1ccc(C(=O)O)cc1S(=O)(=O)NC] | 3.6 | 8% | 6% |
| 25 | 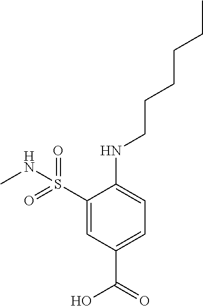 [SMILES: CCCCCCNc1ccc(C(=O)O)cc1S(=O)(=O)NC] | 3.7 | Inactive | 7% |
| 26 | 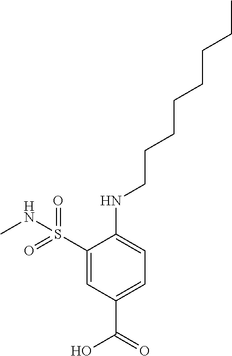 [SMILES: CCCCCCCCNc1ccc(C(=O)O)cc1S(=O)(=O)NC] | 3.8 | 16% | 20% |
| 27 | 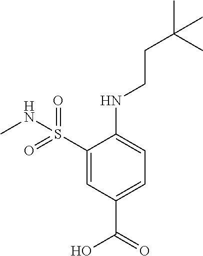 [SMILES: CNS(=O)(=O)c1cc(C(=O)O)ccc1NCCC(C)(C)C] | 3.9 | Inactive | 14% |
| 28 | 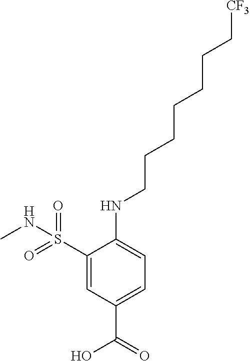 [SMILES: CNS(=O)(=O)c1cc(C(=O)O)ccc1NCCCCCCCC(F)(F)F] | 3.10 | Inactive | 14% |
| 29 | 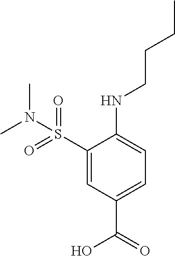 [SMILES: CCCCNc1ccc(C(=O)O)cc1S(=O)(=O)N(C)C] | 3.11 | 9% | 8% |
| 30 | 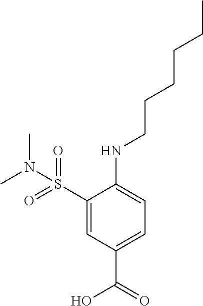 [SMILES: CCCCCCNc1ccc(C(=O)O)cc1S(=O)(=O)N(C)C] | 3.12 | 8% | 13% |
| 31 | 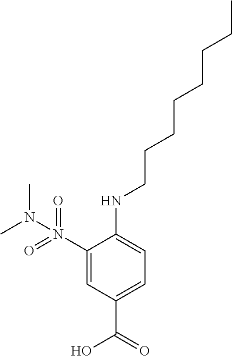 | 3.13 | Inactive | 45% |
| 32 | 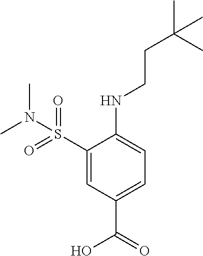 [SMILES: CN(C)S(=O)(=O)c1cc(C(=O)O)ccc1NCCC(C)(C)C] | 3.14 | Inactive | 11% |
| 33 | 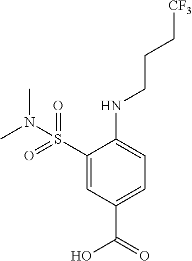 [SMILES: CN(C)S(=O)(=O)c1cc(C(=O)O)ccc1NCCCC(F)(F)F] | 3.15 | 11% | Inactive |
| 34 | 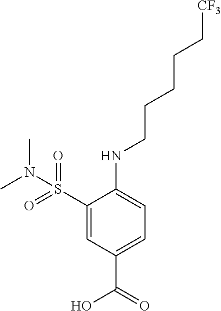 [SMILES: CN(C)S(=O)(=O)c1cc(C(=O)O)ccc1NCCCCCC(F)(F)F] | 3.16 | 17% | Inactive |
| 35 | 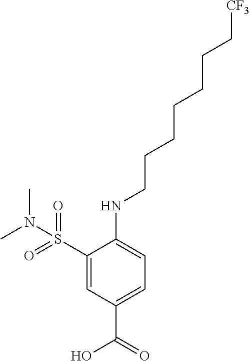 [SMILES: CN(C)S(=O)(=O)c1cc(C(=O)O)ccc1NCCCCCCCC(F)(F)F] | 3.17 | 37% | 46% |
| 36 | 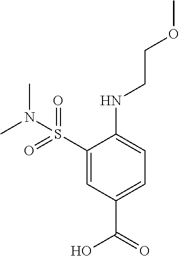 | 3.18 | 13% | Inactive |
| 37 | 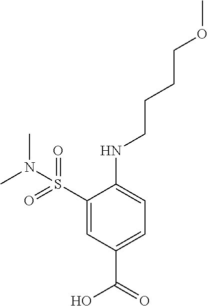 | 3.19 | 6% | Inactive |
| 38 | 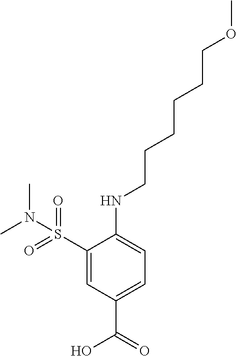 | 3.20 | Inactive | 20% |
| 39 | 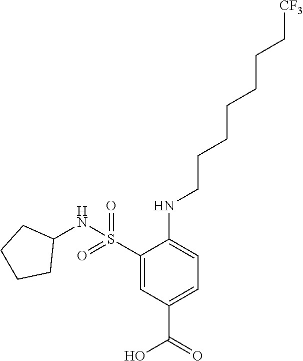 [SMILES: O=C(O)c1ccc(NCCCCCCCC(F)(F)F)c(S(=O)(=O)NC2CCCC2)c1] | 3.21 | 18% | 51% |
| 40 | 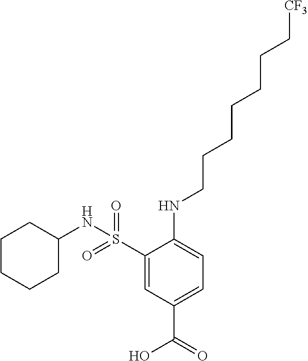 [SMILES: O=C(O)c1ccc(NCCCCCCCC(F)(F)F)c(S(=O)(=O)NC2CCCCC2)c1] | 3.22 | 4% | 54% |
| 41 | 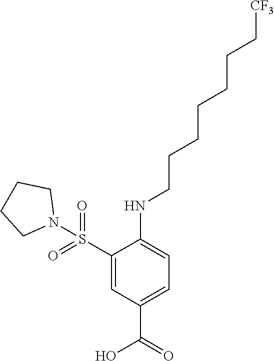 [SMILES: O=C(O)c1ccc(NCCCCCCCC(F)(F)F)c(S(=O)(=O)N2CCCC2)c1] | 5.5 | 2% | 39% |
| 42 | 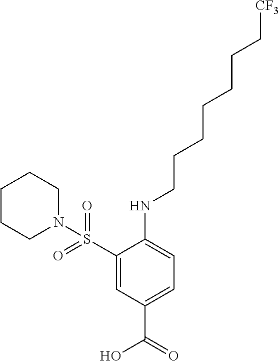 [SMILES: O=C(O)c1ccc(NCCCCCCCC(F)(F)F)c(S(=O)(=O)N2CCCCC2)c1] | 5.6 | Inactive | 55% |
| 43 | 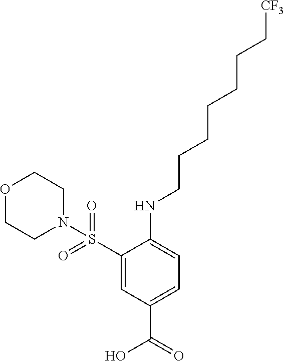 [SMILES: O=C(O)c1ccc(NCCCCCCCC(F)(F)F)c(S(=O)(=O)N2CCOCC2)c1] | 5.7 | 13% | 24% |
| 44 | 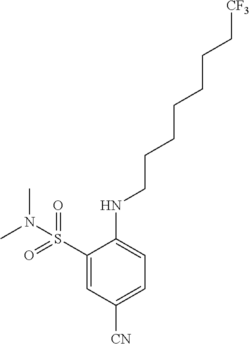 [SMILES: CN(C)S(=O)(=O)c1cc(C#N)ccc1NCCCCCCCC(F)(F)F] | 6.3 | 20% | n.a |
| 45 | 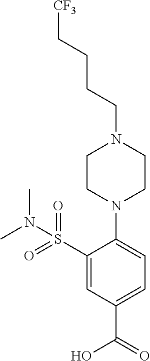 [SMILES: CN(C)S(=O)(=O)c1cc(C(=O)O)ccc1N1CCN(CCCCC(F)(F)F)CC1] | 9.1 | 3% | n.a |
| 46 | 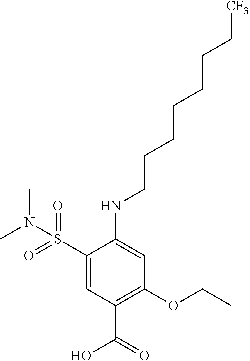 [SMILES: CCOc1cc(NCCCCCCCC(F)(F)F)c(S(=O)(=O)N(C)C)cc1C(=O)O] | 12.9 | not available | 12.2% |
| 47 | 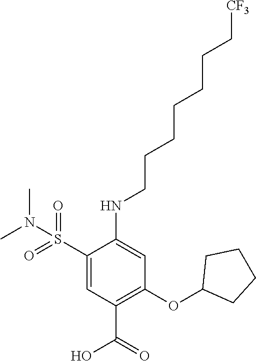 [SMILES: CN(C)S(=O)(=O)c1cc(C(=O)O)c(OC2CCCC2)cc1NCCCCCCCC(F)(F)F] | 12.10 | not available | 26.6% |
| 48 | 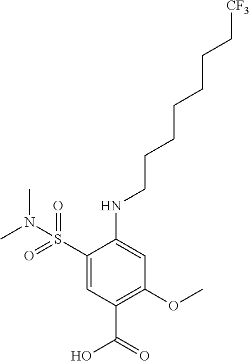 [SMILES: COc1cc(NCCCCCCCC(F)(F)F)c(S(=O)(=O)N(C)C)cc1C(=O)O] | 13.1 | not available | 14.2% |
| 49 | 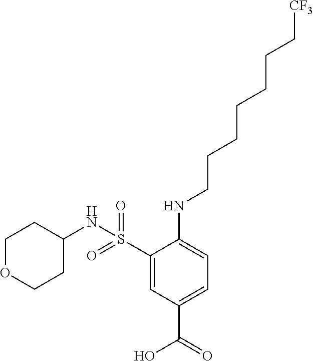 [SMILES: O=C(O)c1ccc(NCCCCCCCC(F)(F)F)c(S(=O)(=O)NC2CCOCC2)c1] | 14.3 | not available | 7.1% |
| 50 | 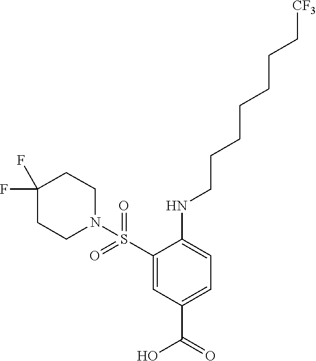 [SMILES: O=C(O)c1ccc(NCCCCCCCC(F)(F)F)c(S(=O)(=O)N2CCC(F)(F)CC2)c1] | 14.4 | not available | 48.0% |
| 51 | 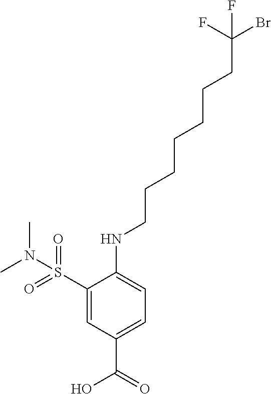 [SMILES: CN(C)S(=O)(=O)c1cc(C(=O)O)ccc1NCCCCCCCC(F)(F)Br] | 15.1 | not available | 17.2% |

According to one embodiment of the present invention, the most active compounds are: compound 1.7, 1.17, 2.2, 2.6, 2.7, 2.8, 2.9, 3.6, 3.7, 3.8, 3.9, 3.10, 3.11, 3.12, 3.13, 3.14, 3.17, 3.20, 3.21, 3.22, 5.5, 5.6, 5.7, 13.1, 14.4, 15.1.

Chloride Kinetic Assay

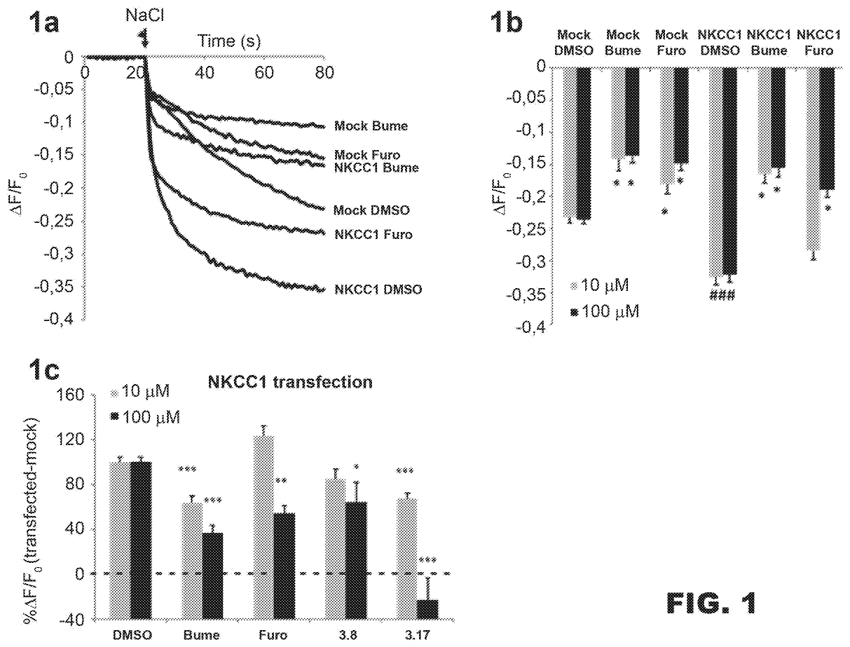

To screen in vitro the compounds efficiency in blocking NKCC1, a functional NKCC1 transporter assay was performed by measuring variation of Cl⁻ ion concentration in the cell through a Cl⁻ sensitive membrane-tagged yellow fluorescent protein (mbYFPQS, Addgene). mbYFPQS fluorescence is inversely dependent on the concentration of Cl⁻ inside the cell thus allowing an indirect estimation of the Cl⁻ transporter activity. In particular, HEK293 cells were transfected with NKCC1 or mock construct (control) together with the Cl⁻ sensitive YFP. After 2 DIV, the cells were treated with bumetanide and furosemide (as positive controls) or with each of the tested compounds of the invention in a Cl⁻ free medium. After 30 min, the inhibitory activity of the compounds was tested by monitoring fluorescence upon application of NaCl (FIG. 1a). Transported by NKCC1, Cl binds the YFP, leading to a fluorescence decrease. NKCC1⁻-transfected cells showed a strong decrease in fluorescence levels upon NaCl application, compared to mock-transfected cells (FIG. 1b). Pre-incubation with bumetanide at 10 μM and 100 μM significantly reduced this effect, whereas pre-incubation with furosemide was effective at 100 μM only (FIG. 1b). Moreover, the data were again normalized due to the decrease in fluorescence observed in the mock-transfected cells upon application of bumetanide or furosemide. With the Cl kinetic assay, the NKCC1 inhibitory activity of the selected compounds was tested (FIG. 1c). Notably, at 100 μM, compound 3.17 inhibited NKCC1 better than bumetanide and furosemide.

Calcium Kinetic Assay

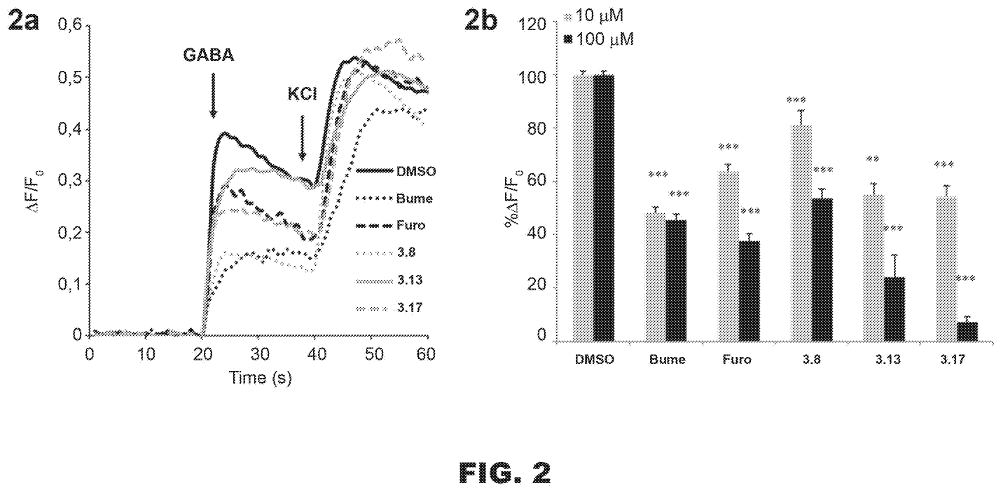

Next, the compounds of the invention were tested for their ability to revert the depolarizing GABAergic signaling in immature neurons. This effect was indirectly measured as calcium influx into the cells with an in vitro calcium kinetic assay in primary cultures of hippocampal neurons. The calcium kinetic assay exploits the physiological, endogenous, high expression of NKCC1 in immature neurons, which causes depolarizing actions of GABA and can activate voltage-gated Ca²⁺ channels. Thus, in immature neurons, a compound that blocks NKCC1 is predicted to inhibit Ca²⁺ responses upon GABA application. Immature neurons were cultured for 3 days in vitro (3 DIV) and loaded for 15 min with a calcium-sensitive dye (Fluo4). Then, the neurons were treated with bumetanide and furosemide (as positive controls) or with each of the selected compounds for 15 min. As a functional readout, the fluorescence level was monitored in these cultures before and after application of GABA (100 μM, for 20 sec). To test for neuronal viability at the end of the experiment, KCl was applied (90 mM, for 40 sec), which strongly depolarizes neurons, causing high activation of voltage-gated Ca²⁺ channels in live cells. To quantify how bumetanide, furosemide, and selected compounds influenced NKCC1 inhibition, the fluorescence values were normalized upon GABA application to the fluorescence levels upon KCl application in treated neurons. Bumetanide, furosemide, and each of the selected compounds significantly reduced the fluorescence increase upon GABA application compared with vehicle (DMSO)-treated controls. They did not affect fluorescence levels upon KCl application (FIG. 2a). The selected compounds displayed optimal potency in inhibiting the Ca²⁺ response upon GABA stimulus (FIG. 2b, with fluorescence values comparable to bumetanide at 10 μM, but even better than bumetanide at 100 μM, in agreement with the chloride (YFP) assay.

Pharmacodynamics Studies

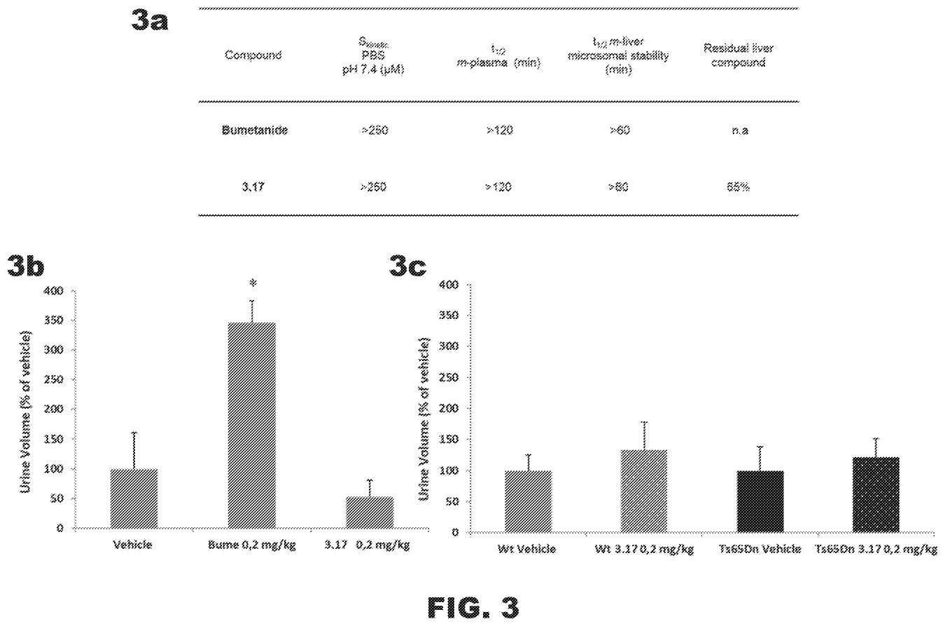

The selected NKCC1 inhibitor compound 3.17 has been evaluated for solubility in aqueous buffers, and stability in plasma and phase I metabolism in vitro (FIG. 3a). The compound was highly soluble (>250 μM in PBS, pH 7.4), and highly resistant to hydrolysis and phase I metabolism (t½>120 min in plasma and t½>60 min in liver microsomes). The data demonstrate the compound as a promising solubility and metabolic stability in vitro.

Cognitive Impairment Test

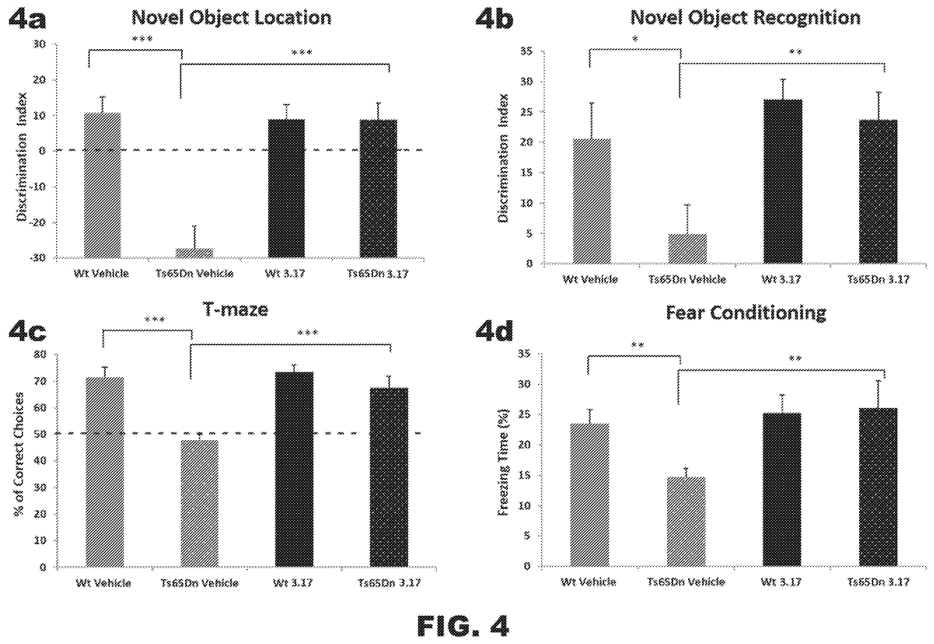

The efficacy of compound 3.17 in rescuing cognitive impairment in four different cognitive tests in Ts65Dn mice (FIG. 4) has been evaluated. Adult Ts65Dn mice and their WT littermates were treated (2 months old) for one week with 3.17 (i.p. 0.2 mg/kg) or its vehicle. For the following three weeks, the animals were tested in four different tasks assessing memory and cognition: a) novel object location task (Deidda, G. et al. Reversing excitatory GABAAR signaling restores synaptic plasticity and memory in a mouse model of Down syndrome. Nat Med 2015, 21 (4), 318-26; Contestabile, A. et al. Lithium rescues synaptic plasticity and memory in Down syndrome mice. J Clin Invest 2013, 123 (1), 348-61), b) novel object recognition test (Deidda G. 2015; Fernandez, F., Garner, C. C., Object recognition memory is conserved in Ts1Cje, a mouse model of Down syndrome. Neuroscience letters 2007, 421, 137-141), c) T-maze task (Belichenko, N. P. et al. The “Down syndrome critical region” is sufficient in the mouse model to confer behavioral, neurophysiological, and synaptic phenotypes characteristic of Down syndrome. J Neurosci 2009, 29 (18), 5938-48) (spontaneous alteration protocol, 11 trials) and d) fear conditioning test (Deidda G. 2015; Costa, A. C. et al. Acute injections of the NMDA receptor antagonist memantine rescue performance deficits of the Ts65Dn mouse model of Down syndrome on a fear conditioning test. Neuropsychopharmacology 2008, 33 (7), 1624-32). As expected, Ts65Dn mice treated with the vehicle showed a decreased performance in comparison to WT. Treatment with 3.17 ameliorated the cognitive performance of Ts65Dn mice (FIG. 4).

### Example 3: NKCC1 vs. NKCC2 Selectivity Data

The compounds of the invention were tested for selective inhibition of NKCC1 compared to NKCC2, as reported in table 2 below.

TABLE 2

| | | NKCC1 | NKCC1 | |
|---|---|---|---|---|
| | | Inhibition | Inhibition | NKCC2 |
| | | % 10 μM | % 10 μM | Inhibition |
| | Compound | HEK Chloride | Neurons Calcium | % 10 μM |
| Entry | id | YFP assay | kinetic assay | Thallium Assay |
| 1 | Bumetanide | 54% | 52% | 99% |
| 2 | Furosemide | Inactive | 36% | 0% |
| 3 | 1.6 | 11% | n.a | 4% |
| 4 | 1.7 | 25% | 36% | 10% |
| 5 | 1.8 | 12% | n.a. | 10% |
| 6 | 1.9 | 5% | n.a | 0% |
| 7 | 1.10 | Inactive | n.a | 0% |
| 8 | 1.11 | 3.4% | n.a | 0% |
| 9 | 1.12 | Inactive | n.a | 0% |
| 10 | 1.13 | 17% | n.a | 0% |
| 11 | 1.14 | Inactive | n.a | 0% |
| 12 | 1.15 | 16% | n.a | 0% |
| 13 | 1.16 | Inactive | n.a | 0% |
| 14 | 1.17 | Inactive | n.a | 13% |
| 15 | 2.2 | 36% | 2% | 0% |
| 16 | 2.3 | 10% | n.a | 14% |
| 17 | 2.4 | 14% | n.a | 16% |
| 18 | 2.5 | Inactive | n.a | 0% |
| 19 | 7.4 | 30% | n.a | n.a |
| 20 | 2.6 | Inactive | 14% | 0% |
| 21 | 2.7 | 4% | 10% | 0% |
| 22 | 2.8 | 5% | 3% | 0% |
| 23 | 2.9 | 1% | 25% | 23% |
| 24 | 3.6 | 8% | 6% | 22% |
| 25 | 3.7 | Inactive | 7% | 8% |
| 26 | 3.8 | 16% | 20% | 0% |
| 27 | 3.9 | Inactive | 14% | 12% |
| 28 | 3.10 | Inactive | 14% | n.a |
| 29 | 3.11 | 9% | 8% | 0% |
| 30 | 3.12 | 8% | 13% | 29% |
| 31 | 3.13 | Inactive | 45% | 0% |
| 32 | 3.14 | Inactive | 11% | 0% |
| 33 | 3.15 | 11% | Inactive | n.a |
| 34 | 3.16 | 17% | Inactive | n.a |
| 35 | 3.17 | 37% | 46% | 0% |
| 36 | 3.18 | 13% | Inactive | n.a |
| 37 | 3.19 | 6% | Inactive | n.a |
| 38 | 3.20 | Inactive | 20% | n.a |
| 39 | 3.21 | 18% | 51% | n.a |
| 40 | 3.22 | 4% | 54% | n.a |
| 41 | 5.5 | 2% | 39% | n.a |
| 42 | 5.6 | Inactive | 55% | n.a |
| 43 | 5.7 | 13% | 24% | n.a |
| 44 | 6.3 | 20% | n.a | n.a |
| 45 | 9.1 | 3% | n.a | n.a |
| 46 | 10.1 | n.a | n.a | n.a |

As per the exemplified data reported in Table 2 above, some of the compounds show a better NKCC1/NKCC2 selectivity.

As an advantage, the compounds do not have a diuretic side-effect.

In particular, said advantage has been shown for the compounds 1.7, 1.15, 2.2, 2.6, 2.7, 2.8, 3.8, 3.13, 3.14 and 3.17, which are particularly preferred within the present invention.

In Vitro Thallium-Based Assay in HEK Cells

The Thallium-based assay is a standard assay used to measure activity of potassium transporters, like NKCC2 which is a sodium potassium and chloride co-transporter. The assay consists on the monitoring of the cells upon the application of thallium (which mimic K⁺) and consequently NaCl, which entering into cells by NKCC2, activated by the presence of the chloride ions, binds the fluorescent dye, thus determining a fluorescence increase. This assay involves parallel testing in 96 wells for a quick and easy drug screening. In detail, kidney epithelial cells (HEK293) were transfected with NKCC2 transporters, or a mock construct (control). After two days, the cells were loaded with a thallium-sensitive fluorescent dye in a Cl-free medium. After 1 hour of incubation, the inhibitory activity of bumetanide and furosemide (as positive controls) and the new compounds by monitoring fluorescence upon application of thallium (to mimic K) and subsequently NaCl were tested. When entering cells by NKCC2 (activated by the presence of Cl), thallium binds the fluorescent dye and increases fluorescence. Upon application of thallium, NKCC2-transfected cells showed a strong increase in fluorescence levels compared to mock-transfected cells. Pre-incubation with bumetanide (10 μM) significantly reduced the ion flux and the consequent increase in fluorescence NKCC2-transfected cells. A decreased fluorescence in the mock-transfected cells treated with bumetanide and furosemide was observed. This indicates that the HEK293 cells express endogenous transporters that are sensitive to bumetanide/furosemide. This latter result was used to normalize the fluorescence measurements obtained with the assay. In particular, the ΔF/F0 value of the mock-transfected cells (both control and treated) was subtracted from the respective ΔF/F0 value of the cells transfected with the Cl transporters. With this assay, the novel chemical entities were tested for their ability to block NKCC2 (Results in Table 2).

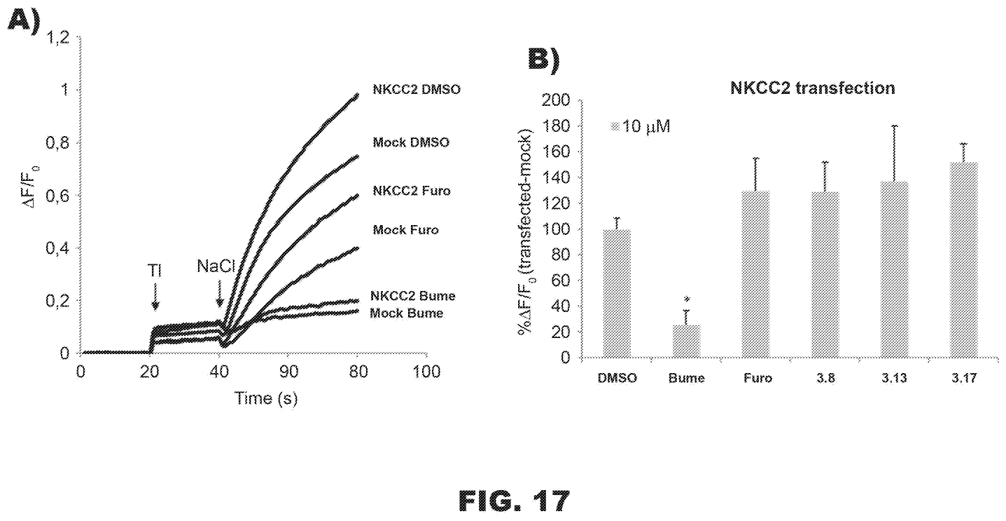

FIG. 17 shows the results of the thallium assays: a) Examples traces obtained in the thallium-based assay on untrasfected (mock) or NKCC2-transfected kidney epithelial (HEK293) cells. The arrow indicates the addition of thallium (final concentration 2 mM) and NaCl stimulus (135 mM) used to initiate the flux assay. b) Quantification of the effect of bumetanide, furosemide and 3 example compounds (3.8, 3.13, 3.17) in the thallium-based assay on NKCC2-transfected HEK293 cells. Data represents mean±sem from 5 independent experiments, and they are represented as % of the controls. *P<0.05, **P<0.01, ***P<0.001 Kruskal-Wallis Anova (Dunn's Post hoc Test); ###P<0.001 two-tailed unpaired Student t-test.

VPA Autism Model

In vivo assessment of the efficacy of the selected NKCC1 inhibitor in the valproic acid (VPA)-induced mouse model of autism, to assess its ability to rescue altered social interaction. The VPA model was obtained by treating pregnant C57bl/6j dams at 12.5 days of pregnancy with 600 mg/kg (i.p.) of VPA dissolved in PBS. VPA-treated dams give birth to offspring that exhibits behaviors related to core symptoms of autism (Nicolini and Fahnestock, 2018). As control, the offspring of C57bl/6j dams treated at 12.5 with PBS was used. To assess the efficacy of the compound to restore social deficits, juvenile male offspring of both the VPA- and PBS-treated dams were treated (i.p injection) with 0.2 mg/kg of compound 3.17 dissolved in PBS or 2% DMSO dissolved in PBS as control for seven days. Then, mice were tested for their social ability and for repetitive behaviors in different tests. The social ability was tested in the three-chamber test (Silverman et al., 2010). In the three-chamber test, mice are singularly placed in a three-chamber box with openings between the chambers. After ten minutes of free exploration, a never-before-met intruder is placed under one pencil cup in one chamber and an empty pencil cup was placed in the other chamber. The sociability index consists in the time in which the animal explore the never-before-met intruder respect the time in which the animal explore the the pencil cup and it is defined as: [(time spent with intruder−time spent with empty cup)/(time spent with intruder+time spent with empty cup) %]. In a second phase a new intruder was placed under the previously empty pencil case in order to measure the social novelty index, i.e. the time of exploration of the new intruder compared to the already encountered subject in the previous 10 minutes. The social novelty index is measured as follows: [(time spent with the new intruder−time spent with the old intruder)/(time spent with the new intruder+time spent with the old intruder) %].

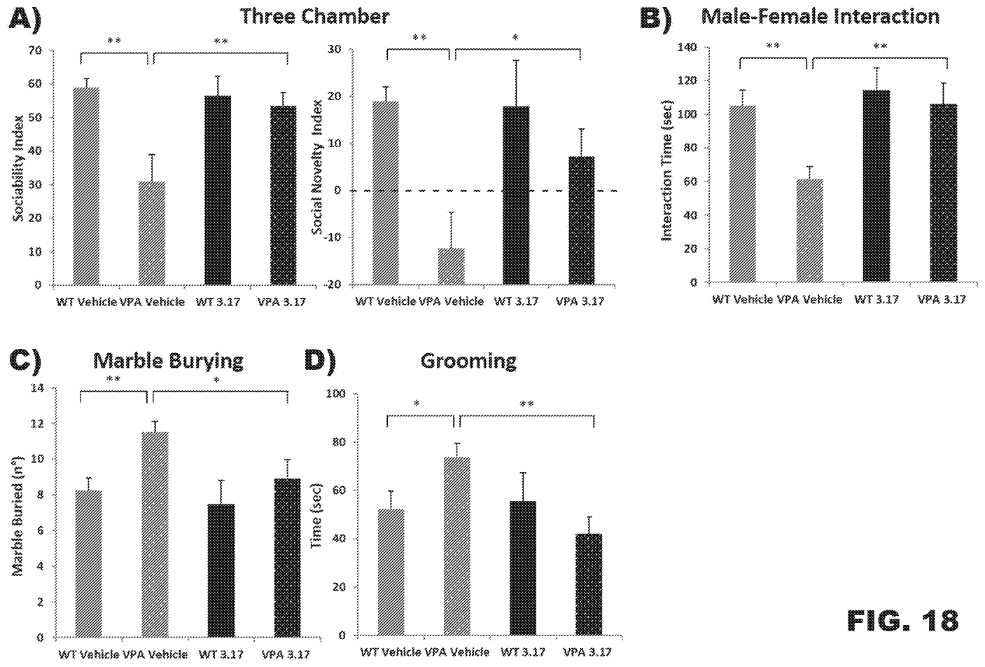

As reported in FIG. 18A, VPA mice treated with vehicle showed a significant lower sociability index and social novelty when compared to the naive mice treated with vehicle. The treatment with the compound 3.17 in VPA mice completely restored the sociability index and the social novelty index to the control level.

The sociability during male-female interaction was then assessed (Drapeau et al., 2018). In this test the tested mouse, after 5 minutes of habituation, is evaluated for its approach to a female intruder mouse that is placed for 5 minutes in the same cage. The time spent interacting is calculated as a measure of male-female social interaction. As shown in FIG. 18B, vehicle-treated VPA mice showed a significantly lower male-female interaction index than vehicle-treated naive mice. Treatment with the compound 3.17 in VPA mice completely restored the interaction. Finally, repetitive behaviors were evaluated in two different tests. In the marble burying test (Eissa et al., 2018) the mouse is placed in a cage with 4 cm of litter on top of which 15 (5*3) balls are neatly placed. The repetitive behavior is evaluated as the number of marbles buried in the litter. The grooming test consists in the assessment of the grooming behavior, i.e. licking or scratching the head or other parts of the body with the front legs, typical behavior of rodents (Campolongo et al., 2018). During the test, the mouse is placed in a cylindrical support and after 10 minutes of habituation, the repetitive grooming activity is measured during 5 minutes. As shown in FIGS. 18C and 18D, vehicle-treated VPA mice showed more repetitive behavior (more marbles buried and more time spent grooming) than vehicle-treated naive mice. Treatment with compound 3.17 in VPA mice completely restored repetitive behaviors at the control level.

1. A compound having Formula Ia or a pharmaceutically acceptable salt thereof or stereoisomeric forms thereof, or the individual geometrical isomers, enantiomers, diastereoisomers, tautomers, zwitterions and pharmaceutically acceptable salts thereof: 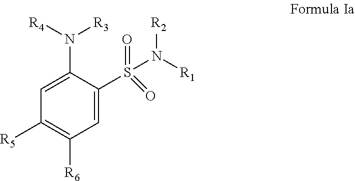
wherein:
R₁ and R₂ are independently selected from the group consisting of hydrogen; linear or branched, C₁₋₁₀ alkyl optionally comprising one or more unsaturations and optionally substituted with a substituent selected from the group consisting of halogens, —OH, —C₃₋₈cycloalkyl, non-aromatic heterocycles, aromatic heterocycles, —C₁₋₆ alkoxyalkyl, —NH₂, —NO₂, amides, carboxylic acids, ketones, ethers, esters, aldehydes, or sulfonamides; linear or branched substituted or unsubstituted C₃₋₈ cycloalkyl; linear or branched substituted or unsubstituted C₄₋₁₀ cycloalkylalkyl; C₃₋₈ heterocycle; and optionally substituted phenyl;
or R₁ and R₂, together with the nitrogen atom to which they are attached, form a substituted or unsubstituted saturated heterocycle;
R₃ and R₄ are independently selected from the group consisting of hydrogen; linear or branched C₁₋₁₀ alkyl optionally comprising one or more unsaturations and optionally substituted with a substituent selected from the group consisting of halogens, —OH, —C₃₋₈cycloalkyl, non-aromatic heterocycles, aromatic heterocycles, —C₁₋₆ alkoxyalkyl, —NH₂, —NO₂, amides, carboxylic acids, ketones, ethers, esters, aldehydes, or sulfonamides; C₃₋₁₀ cycloalkyl; C₄₋₁₀ cycloalkylalkyl; C₂₋₈ haloalkyl; linear or branched, unsubstituted or substituted C₂₋₈ heteroalkyl; and optionally substituted phenyl provided that at least one of R₃ and R₄ is other than hydrogen;
or R₃ and R₄, when taken together with the nitrogen atom to which they are attached, form a substituted or unsubstituted saturated heterocycle;
R₅ is selected from the group consisting of hydrogen; halogen; hydroxyl; —O—C₁₋₁₀ alkyl; —O—C₃₋₁₀ cycloalkyl; —O—C₃₋₈ heterocycloalkyl; C₁₋₁₀ alkoxyalkyl; C₃₋₁₀ alkoxycycloalkyl; optionally substituted phenoxyl; —NH₂; C₁₋₈ alkylamine; C_{2-C16} dialkylamine; aniline; —SH; C₁₋₈ alkylthioether; thiophenol; and —NO₂;
R₆ is selected from the group consisting of nitro; nitrile; —CH₂OH; carboxylic acid; C₁₋₄ alkyl ester; C₂₋₈ heteroalkyl ester; C₃₋₆ cycloalkyl ester; phenyl ester; carboxamide; cyclic amide; tetrazole;
wherein when R₆ is nitro, the following conditions are satisfied at the same time:
R₁ is other than H,
R₂ is other than linear or branched unsubstituted C₂₋₆ alkyl,
R₃ is other than H,
R₄ is other than linear and unsubstituted C₁₋₃ alkyl and
R₅ other than H;
and provided that the compound of formula Ia is not one of the following:

| R₁ | R₂ | R₃ | R₄ | R₅ | R₆ |
|---|---|---|---|---|---|
| H | (CH₂)₃—OCH₃ | H | CH₂CH₃ | H | —NO₂ |
| H | CH₂CH(CH₃)—OCH₃ | H | CH₂CH₃ | H | —NO₂ |
| H | (CH₂)₂—OCH₃ | H | CH₂CH₃ | H | —NO₂ |
| H | (CH₂)₂—OCH₃ | H | CH₂CH₂CH₃ | H | —NO₂ |
| H | CH(CH₃)CH₂—OCH₃ | H | CH₂CH₃ | H | —NO₂ |
| H | CH₃ | H | CH₂CH₂OH | H | COOH |
| H | CH₃ | H | CH₂CH₃ | H | COOH |
| H | CH₃ | H | CH₂CH₂CH₂CH₃ | H | COOH |
| H | phenyl | H | cyclohexyl | H | COOH |

2. The compound according to claim 1, wherein R₁ and R₂ are independently selected from the group consisting of hydrogen; linear or branched, C₁₋₁₀ alkyl optionally comprising one or more unsaturations and optionally substituted with a substituent selected from the group consisting of halogens, —OH, —C₃₋₈cycloalkyl, non-aromatic heterocycles, aromatic heterocycles, —C₁₋₆ alkoxyalkyl, —NH₂, —NO₂, amides, carboxylic acids, ketones, ethers, esters, aldehydes, or sulfonamides: linear or branched substituted or unsubstituted C₃₋₈ cycloalkyl; linear or branched substituted or unsubstituted C₄₋₁₀ cycloalkylalkyl; and optionally substituted phenyl;
or R₁ and R₂, together with the nitrogen atom to which they are attached, form a substituted or unsubstituted saturated heterocycle;
R₃ and R₄ are independently selected from the group consisting of hydrogen; linear or branched C₁₋₁₀ alkyl optionally comprising one or more unsaturations and optionally substituted with a substituent selected from the group consisting of halogens, —OH, —C₃₋₈cycloalkyl, non-aromatic heterocycles, aromatic heterocycles, —C₁₋₆ alkoxyalkyl, —NH₂, —NO₂, amides, carboxylic acids, ketones, ethers, esters, aldehydes, or sulfonamides; C₃₋₁₀ cycloalkyl; C₄₋₁₀ cycloalkylalkyl; C₂₋₈ haloalkyl; linear or branched, unsubstituted or substituted C₂₋₈ heteroalkyl; and optionally substituted phenyl; provided that at least one of R₃ and R₄ is other than hydrogen;
or R₃ and R₄, when taken together with the nitrogen atom to which they are attached, form a substituted or unsubstituted saturated heterocycle;
R₅ is selected from the group consisting of hydrogen; halogen; hydroxyl; C₁₋₁₀ alkoxyalkyl; C₃₋₁₀ alkoxycycloalkyl; optionally substituted phenoxyl; —NH₂; C₁₋₈ alkylamine; C_{2-C16} dialkylamine; aniline; —SH; C₁₋₈ alkylthioether; thiophenol; and —NO₂;
R₆ is selected from the group consisting of nitro; nitrile; —CH₂OH; carboxylic acid; C₁₋₄ alkyl ester; C₂₋₈ heteroalkyl ester; C₃₋₆ cycloalkyl ester; phenyl ester; carboxamide; cyclic amide; and tetrazole.

3. The compound according to claim 1, wherein R₁ and R₂ are independently selected from H, —CH₃, cyclopentane, cyclohexane, 4-tetrahydropirane or, together with the nitrogen atom to which they are attached are a morpholine, a piperidine optionally substituted with at least one halogen, a pirrolidine.

4. The compound according to claim 1, wherein R₃ and R₄ are independently selected from hydrogen, linear or branched —C₁₋₈ alkyl optionally substituted with one C₁₋₆ alkoxyalkyl, —C₂₋₈ haloalkyl, or R₃ and R₄, when taken together with the nitrogen atom to which they are attached, are a substituted or unsubstituted saturated heterocycle.

5. The compound according to claim 1, wherein hydrogen atoms on cycloalkyl are substituted by groups selected from: halogens, —OH, —C₃₋₈cycloalkyl, non-aromatic heterocycles, aromatic heterocycles, —C₁₋₆alkoxyalkyl, —NH₂, —NO₂, amides, ethers, esters, carboxylic acids, aldehydes, ketones, or sulfonamides groups.

6. The compound according to claim 1, wherein heterocycles are substituted with halogens, —C₁₋₅alkyl, —C₁₋₅alkenyl, or —C₁₋₅haloalkyl.

7. The compound according to claim 1 wherein the compound is selected from the group consisting of:
2-(butylamino)-5-nitro-benzenesulfonamide,
2-(hexylamino)-5-nitro-benzenesulfonamide,
5-nitro-2-(octylamino)benzenesulfonamide,
2-(3,3-dimethylbutylamino)-5-nitro-benzenesulfonamide,
2-(butylamino)-N-methyl-5-nitro-benzenesulfonamide,
2-(hexylamino)-N-methyl-5-nitro-benzenesulfonamide,
2-N-methyl-5-nitro-2-(octylamino)benzenesulfonamide,
2-(3,3-dimethylbutylamino)-N-methyl-5-nitro-benzenesulfonamide,
2-(butylamino)-N,N-dimethyl-5-nitro-benzenesulfonamide,
2-(hexylamino)-N,N-dimethyl-5-nitro-benzenesulfonamide,
N,N-dimethyl-5-nitro-2-(octylamino)benzenesulfonamide,
2-(3,3-dimethylbutylamino)-N,N-dimethyl-5-nitro-benzenesulfonamide,
4-(butylamino)-2-chloro-5-sulfamoyl-benzoic acid,
2-chloro-4-(hexylamino)-5-sulfamoyl-benzoic acid,
2-chloro-4-(octylamino)-5-sulfamoyl-benzoic acid,
2-chloro-4-(3,3-dimethylbutylamino)-5-sulfamoyl-benzoic acid,
4-(butylamino)-3-sulfamoyl-benzoic acid,
4-(hexylamino)-3-sulfamoyl-benzoic acid,
4-(octylamino)-3-sulfamoyl-benzoic acid,
4-(3,3-dimethylbutylamino)-3-sulfamoyl-benzoic acid,
4-(butylamino)-3-(methylsulfamoyl)benzoic acid,
4-(hexylamino)-3-(methylsulfamoyl)benzoic acid,
3-(methylsulfamoyl)-4-(octylamino)benzoic acid,
4-(3,3-dimethylbutylamino)-3-(methylsulfamoyl)benzoic acid,
3-(methylsulfamoyl)-4-(8,8,8-trifluorooctylamino)benzoic acid,
4-(butylamino)-3-(dimethylsulfamoyl)benzoic acid,
3-(dimethylsulfamoyl)-4-(hexylamino)benzoic acid,
3-(dimethylsulfamoyl)-4-(octylamino)benzoic acid,
4-(3,3-dimethylbutylamino)-3-(dimethylsulfamoyl)benzoic acid,
3-(dimethylsulfamoyl)-4-(4,4,4 trifluorobutylamino) benzoic acid,
3-(dimethylsulfamoyl)-4-(6,6,6-trifluorohexylamino) benzoic acid,
3-(dimethylsulfamoyl)-4-(8,8,8-trifluorooctylamino) benzoic acid,
3-(dimethylsulfamoyl)-4-(2-methoxyethylamino)benzoic acid,
3-(dimethylsulfamoyl)-4-(4-methoxybutylamino)benzoic acid,
3-(dimethylsulfamoyl)-4-(6-methoxyhexylamino)benzoic acid,
3-(cyclopentylsulfamoyl)-4-(8,8,8-trifluorooctylamino) benzoic acid,
3-(cyclohexylsulfamoyl)-4-(8,8,8-trifluorooctylamino) benzoic acid,
3-pyrrolidin-1-ylsulfonyl-4-(8,8,8-trifluorooctylamino) benzoic acid,
3-(1-piperidylsulfonyl)-4-(8,8,8-trifluorooctylamino) benzoic acid,
3-morpholinosulfonyl-4-(8,8,8-trifluorooctylamino) benzoic acid,
5-cyano-N,N-dimethyl-2-(8,8,8-trifluorooctylamino) benzenesulfonamide,
2-hydroxy-5-sulfamoyl-4-(8,8,8-trifluorooctylamino) benzoic acid,
3-(dimethylsulfamoyl)-4-[4-(5,5,5 trifluoropentyl)piperazin-1-yl] benzoic acid,
N,N-dimethyl-5-(1H-tetrazol-5-yl)-2 (8,8,8-trifluorooctylamino)benzenesulfonamide,
Methyl 5-(N,N-dimethylsulfamoyl)-2-methoxy-4-((8,8,8-trifluorooctyl)amino)benzoate,
Methyl 5-(N,N-dimethylsulfamoyl)-2-hydroxy-4-((8,8,8-trifluorooctyl)amino)benzoate,
Methyl 5-(N,N-dimethylsulfamoyl)-2-ethoxy-4-((8,8,8-trifluorooctyl)amino)benzoate,
Methyl 2-(cyclopentyloxy)-5-(N,N-dimethylsulfamoyl)-4-((8,8,8-trifluorooctyl)amino)benzoate,
5-(N,N-dimethylsulfamoyl)-2-ethoxy-4-((8,8,8-trifluorooctyl)amino)benzoic acid,
2-(cyclopentyloxy)-5-(N,N-dimethylsulfamoyl)-4-((8,8,8-trifluorooctyl)amino)benzoic acid,
5-(N,N-dimetylsulfamoyl)-2-methoxy-4-((8,8,8-trifluorooctyl)amino)benzoic acid,
3-morfolinosulfonyl-4-((8,8,8-trifluorooctyl)amino)benzoic acid,
3-((4,4-difluoropiperidin-1-yl)sulfonyl)-4-((8,8,8-trifluorooctyl)amino)benzoic acid,
3-(dimethylsulfamoyl)-4-(hept-6-enylamino)benzoic acid,
Methyl 3-(N,N-dimethylsulfamoyl)-4-(hept-6-en-1-ylamino)benzoate,
Methyl 4-((8-bromo-8,8-difluorooctyl)amino)-3-(N,N-dimethylsulfamoyl)benzoate,
4-[(8-bromo-8,8-difluorooctyl)amino]-3-(dimethylsulfamoyl)benzoic acid,
5-(dimethylsulfamoyl)-2-isopropoxy-4-(8,8,8-trifluorooctylamino)benzoic,
2-(cicloexoxy)-5-(dimethylsulfamoyl)-4-(8,8,8-trifluorooctylamino)benzoic acid,
5-(dimethylsulfamoyl)-2-tetrahydropiran-4-yloxy-4-(8,8,8-trifluorooctylamino)benzoic,
2-(cyclobutoxy)-5-(dimethylsulfamoyl)-4-(8,8,8-trifluorooctylamino)benzoic acid,
Acido 5-(dimethylsulfamoyl)-2-(oxetan-3-yloxy)-4-(8,8,8-trifluorooctylamino)benzoic acid,
5-(dimethylsulfamoyl)-2-(4-piperidyloxy)-4-(8,8,8-trifluorooctylamino)benzoic acid, and
Acido 5-(dimethylsulfamovll-2-fenoxy-4-(8.8.8-trifluorooctylamino)benzoic acid.

8. The compound according to claim 1, the compound selected from the group consisting of:
2-(hexylamino)-5-nitro-benzenesulfonamide,
2-(3,3-dimethylbutylamino)-N,N-dimethyl-5-nitro-benzenesulfonamide,
4-(butylamino)-2-chloro-5-sulfamoyl-benzoic acid,
4-(butylamino)-3-sulfamoyl-benzoic acid,
4-(hexylamino)-3-sulfamoyl-benzoic,
4-(octylamino)-3-sulfamoyl-benzoic acid,
4-(3,3-dimethylbutylamino)-3-sulfamoyl-benzoic acid,
4-(butylamino)-3-(methylsulfamoyl)benzoic acid,
4-(hexylamino)-3-(methylsulfamoyl)benzoic acid,
3-(methylsulfamoyl)-4-(octylamino)benzoic acid,
4-(3,3-dimethylbutylamino)-3-(methylsulfamoyl)benzoic acid,
3-(methylsulfamoyl)-4-(8,8,8-trifluorooctylamino)benzoic acid,
4-(butylamino)-3-(dimethylsulfamoyl)benzoic acid,
3-(dimethylsulfamoyl)-4-(exylamino)benzoic acid,
3-(dimethylsulfamoyl)-4-(octylamino)benzoic acid,
4-(3,3-dimethylbutylamino)-3-(dimethylsulfamoyl)benzoic acid,
3-(dimethylsulfamoyl)-4-(8,8,8-trifluorooctylamino) benzoic acid,
3-(dimethylsulfamoyl)-4-(6-methoxyhexylamino)benzoic acid,
3-(cyclopentylsulfamoyl)-4-(8,8,8-trifluorooctylamino) benzoic acid,
3-(cyclohexylsulfamoyl)-4-(8,8,8-trifluorooctylamino) benzoic acid,
3-pyrrolidin-1-ylsulfonyl-4-(8,8,8-trifluorooctylamino) benzoic acid,
3-(1-piperidylsulfonyl)-4-(8,8,8-trifluorooctylamino) benzoic acid,
3-morpholinosulfonyl-4-(8,8,8-trifluorooctylamino) benzoic acid,
5-(N,N-dimetylsulfamoyl)-2-methoxy-4-((8,8,8-trifluorooctyl)amino)benzoic acid,
3-((4,4-difluoropiperidin-1-yl)sulfonyl)-4-((8,8,8-trifluorooctyl)amino)benzoic acid, and
3-(dimethylsulfamoyl)-4-(hept-6-enylamino)benzoic acid.

9. The compound according to claim 1, the compound selected from the group consisting of:
2-(hexylamino)-5-nitro-benzenesulfonamide,
2-(hexylamino)-N,N-dimethyl-5-nitro-benzenesulfonamide,
4-(butylamino)-2-chloro-5-sulfamoyl-benzoic acid,
4-(butylamino)-3-sulfamoyl-benzoic acid,
4-(hexylamino)-3-sulfamoyl-benzoic acid,
4-(octylamino)-3-sulfamoyl-benzoic acid,
3-(methylsulfamoyl)-4-(octylamino)benzoic acid,
3-(dimethylsulfamoyl)-4-(octylamino)benzoic acid,
(3,3-dimethylbutylamino)-3-(dimethylsulfamoyl)benzoic acid, and
3-(dimethylsulfamoyl)-4-(8,8,8-trifluorooctylamino) benzoic acid.

10. A compound having Formula Ib or a pharmaceutically acceptable salt thereof or stereoisomeric forms thereof, or the individual geometrical isomers, enantiomers, diastereoisomers, tautomers, zwitterions and pharmaceutically acceptable salts thereof: 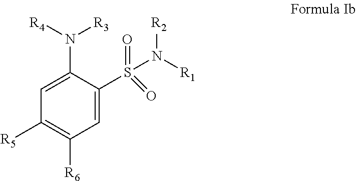
wherein:
R₁ and R₂ are independently are selected from the group consisting of hydrogen; linear or branched, substituted or unsubstituted C₁₋₁₀ alkyl optionally comprising one or more unsaturations; linear or branched substituted or unsubstituted C₃₋₈ cycloalkyl; linear or branched substituted or unsubstituted C₄₋₁₀ cycloalkylalkyl; C₃₋₈ heterocycle; and optionally substituted phenyl;
or R₁ and R₂, together with the nitrogen atom to which they are attached, form a substituted or unsubstituted saturated heterocycle;
R₃ and R₄ are independently selected from the group consisting of hydrogen; substituted or unsubstituted C₁₋₁₀ alkyl optionally comprising one or more unsaturations; C₃₋₁₀ cycloalkyl; C₄₋₁₀ cycloalkylalkyl; C₂₋₈ haloalkyl; linear or branched, unsubstituted or substituted C₂₋₈ heteroalkyl; and optionally substituted phenyl; provided that at least one of R₃ and R₄ is other than hydrogen;
or R₃ and R₄, when taken together with the nitrogen atom to which they are attached, form a substituted or unsubstituted saturated heterocycle;
R₅ is selected from the group consisting of hydrogen; halogen; hydroxyl; —O—C₁₋₁₀ alkyl; —O—C₃₋₁₀ cycloalkyl; —O—C₃₋₈ heterocycloalkyl; C₁₋₁₀ alkoxyalkyl; C₃₋₁₀ alkoxycycloalkyl; optionally substituted phenoxyl; —NH₂; C₁₋₈ alkylamine; C_{2-C16} dialkylamine; aniline; —SH; C₁₋₈ alkylthioether; thiophenol; and —NO₂;
R₆ is selected from the group consisting of nitro; nitrile; —CH₂OH; carboxylic acid; C₁₋₄ alkyl ester; C₂₋₈ heteroalkyl ester; C₃₋₆ cycloalkyl ester; phenyl ester; carboxamide; C₁₋₄ alkyl amide; C₂₋₈ dialkyl amide; cycloalkyl amide; cyclic amide; and tetrazole;
wherein the compound is used in the manufacture of a medicament.

11. A compound having Formula Ic or a pharmaceutically acceptable salt thereof or stereoisomeric forms thereof, or the individual geometrical isomers, enantiomers, diastereoisomers, tautomers, zwitterions and pharmaceutically acceptable salts thereof: 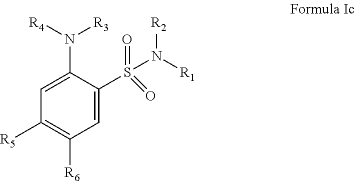
wherein:
R₁ and R₂ are independently selected from the group consisting of hydrogen;
linear or branched, substituted or unsubstituted C₁₋₁₀ alkyl optionally comprising one or more unsaturations;
linear or branched substituted or unsubstituted C₃₋₈ cycloalkyl;
linear or branched substituted or unsubstituted C₄₋₁₀ cycloalkylalkyl;
and optionally substituted phenyl;
or R₁ and R₂, together with the nitrogen atom to which they are attached, form a substituted or unsubstituted saturated heterocycle;
R₃ and R₄ are independently selected from the group consisting of hydrogen; substituted or unsubstituted C₁₋₁₀ alkyl optionally comprising one or more unsaturations; C₃₋₁₀ cycloalkyl; C₄₋₁₀ cycloalkylalkyl; C₂₋₈ haloalkyl; linear or branched, unsubstituted or substituted C₂₋₈ heteroalkyl; and optionally substituted phenyl; provided that at least one of R₃ and R₄ is other than hydrogen;
or R₃ and R₄, when taken together with the nitrogen atom to which they are attached, form a substituted or unsubstituted saturated heterocycle;
R₅ is selected from the group consisting of hydrogen; halogen; hydroxyl; C₁₋₁₀ alkoxyalkyl; C₃₋₁₀ alkoxycycloalkyl; optionally substituted phenoxyl; —NH₂; C₁₋₈ alkylamine; C_{2-C16} dialkylamine; aniline; —SH; C₁₋₈ alkylthioether; thiophenol; and —NO₂;
R₆ is selected from the group consisting of nitro; nitrile; —CH₂OH; carboxylic acid; C₁₋₄ alkyl ester; C₂₋₈ heteroalkyl ester; C₃₋₆ cycloalkyl ester; phenyl ester; carboxamide; C₁₋₄ alkyl amide; C₂₋₈ dialkyl amide; cycloalkyl amide; cyclic amide; and tetrazole;
wherein the compound is used in the manufacture of a medicament.

12. The compound according to claim 10 wherein the compound is used for use in the treatment or in the prevention of pathological conditions associated to depolarizing GABAergic transmission.

13. The compound according to claim 12, wherein said pathological condition is selected from the group consisting of: Down syndrome, neuropathic pain, stroke, cerebral ischemia, cerebral edema, hydrocephalus, traumatic brain injury, Brain Trauma-Induced Depressive-Like Behavior, autism spectrum disorders, autism, Fragile X, Rett, Asperger and DiGeorge syndromes, epilepsy, seizures, epileptic state, West syndrome, glioma, glioblastoma, anaplastic astrocytoma, Parkinson's disease, Hungtinton's disease, schizophrenia, anxiety, Tuberous Sclerosis Complex and associated behavioural problems, and Dravet syndrome.

14. A pharmaceutical composition comprising at least one compound having Formula Ib or a pharmaceutically acceptable salt thereof or stereoisomeric forms thereof, or the individual geometrical isomers, enantiomers, diastereoisomers, tautomers, zwitterions and pharmaceutically acceptable salts thereof: 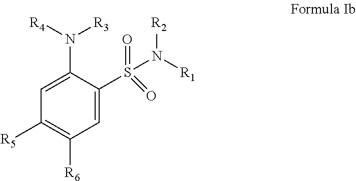
wherein:
R₁ and R₂ are independently selected from the group consisting of hydrogen; linear or branched, substituted or unsubstituted C₁₋₁₀ alkyl optionally comprising one or more unsaturations; linear or branched substituted or unsubstituted C₃₋₈ cycloalkyl; linear or branched substituted or unsubstituted C₄₋₁₀ cycloalkylalkyl; C₃₋₈ heterocycle; and optionally substituted phenyl;
or R₁ and R₂, together with the nitrogen atom to which they are attached, form a substituted or unsubstituted saturated heterocycle;
R₃ and R₄ are independently selected from the group consisting of hydrogen; unsubstituted or substituted C₁₋₁₀ alkyl optionally comprising one or more unsaturations; C₃₋₁₀ cycloalkyl; C₄₋₁₀ cycloalkylalkyl; C₂₋₈ haloalkyl; linear or branched, unsubstituted or substituted C₂₋₈ heteroalkyl; and optionally substituted phenyl; provided that at least one of R₃ and R₄ is other than hydrogen;
or R₃ and R₄, when taken together with the nitrogen atom to which they are attached, form a substituted or unsubstituted saturated heterocycle;
R₅ is selected from the group consisting of hydrogen; halogen; hydroxyl; —O—C₁₋₁₀ alkyl; —O—C₃₋₁₀ cycloalkyl; —O—C₃₋₈ heterocycloalkyl; C₁₋₁₀ alkoxyalkyl; C₃₋₁₀ alkoxycycloalkyl; optionally substituted phenoxyl; —NH₂; C₁₋₈ alkylamine; C_{2-C16} dialkylamine; aniline; —SH; C₁₋₈ alkylthioether; thiophenol; and —NO₂;
R₆ is selected from the group consisting of nitro; nitrile; —CH₂OH; carboxylic acid; C₁₋₄ alkyl ester; C₂₋₈ heteroalkyl ester; C₃₋₆ cycloalkyl ester; phenyl ester; carboxamide; C₁₋₄ alkyl amide; C₂₋₈ dialkyl amide; cycloalkyl amide; cyclic amide; and tetrazole; and
pharmaceutically acceptable excipients.

15. A method for treating disorders associated to depolarizing GABAergic transmission in a mammal in need thereof, the method comprising administering a compound of formula Ib or a pharmaceutically acceptable salt thereof or stereoisomeric forms thereof, or the individual geometrical isomers, enantiomers, diastereoisomers, tautomers, zwitterions and pharmaceutically acceptable salts thereof: 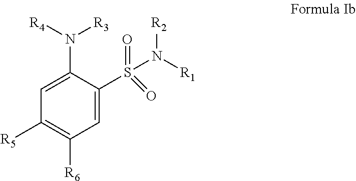
wherein:
R₁ and R₂ are independently selected from the group consisting of hydrogen; linear or branched, substituted or unsubstituted C₁₋₁₀ alkyl optionally comprising one or more unsaturations; linear or branched substituted or unsubstituted C₃₋₈ cycloalkyl; linear or branched substituted or unsubstituted C₄₋₁₀ cycloalkylalkyl; C₃₋₈ heterocycle; and optionally substituted phenyl;
or R₁ and R₂, together with the nitrogen atom to which they are attached, form a substituted or unsubstituted saturated heterocycle;
R₃ and R₄ are independently selected from the group consisting of hydrogen; unsubstituted or substituted C₁₋₁₀ alkyl optionally comprising one or more unsaturations; C₃₋₁₀ cycloalkyl; C₄₋₁₀ cycloalkylalkyl; C₂₋₈ haloalkyl; linear or branched, unsubstituted or substituted C₂₋₈ heteroalkyl; and optionally substituted phenyl; provided that at least one of R₃ and R₄ is other than hydrogen;
or R₃ and R₄, when taken together with the nitrogen atom to which they are attached, form a substituted or unsubstituted saturated heterocycle;
R₅ is selected from the group consisting of hydrogen; halogen; hydroxyl; —O—C₁₋₁₀ alkyl; —O—C₃₋₁₀ cycloalkyl; —O—C₃₋₈ heterocycloalkyl; C₁₋₁₀ alkoxyalkyl; C₃₋₁₀ alkoxycycloalkyl; optionally substituted phenoxyl; —NH₂; C₁₋₈ alkylamine; C_{2-C16} dialkylamine; aniline; —SH; C₁₋₈ alkylthioether; thiophenol; and —NO₂;
R₆ is selected from the group consisting of nitro; nitrile; —CH₂OH; carboxylic acid; C₁₋₄ alkyl ester; C₂₋₈ heteroalkyl ester; C₃₋₆ cycloalkyl ester; phenyl ester; carboxamide; C₁₋₄ alkyl amide; C₂₋₈ dialkyl amide; cycloalkyl amide; cyclic amide; and tetrazole.

16. The compound according to claim 11, wherein the compound is used in the treatment or in the prevention of pathological conditions associated to depolarizing GABAergic transmission.

17. The compound according to claim 16, wherein said pathological condition is selected from the group consisting of Down syndrome, neuropathic pain, stroke, cerebral ischemia, cerebral edema, hydrocephalus, traumatic brain injury, Brain Trauma-Induced Depressive-Like Behavior, autism spectrum disorders, autism, Fragile X, Rett, Asperger and DiGeorge syndromes, epilepsy, seizures, epileptic state, West syndrome, glioma, glioblastoma, anaplastic astrocytoma, Parkinson's disease, Hungtinton's disease, schizophrenia, anxiety, Tuberous Sclerosis Complex and associated behavioural problems, and Dravet syndrome.

18. The composition according to claim 14, wherein the composition further comprises one or more psychoactive drugs and/or anti-inflammatory drugs.
